SEQUENCE LISTING (122 sequences)

SEQ ID NO: 1 - DNA, 29 bp, Artificial Sequence
  rs3794604 primer
acgttggatgatgccctcctggctttgtg

SEQ ID NO: 2 - DNA, 30 bp, Artificial Sequence
  rs3794604 primer
acgttggatgcacttttctagggctttcac

SEQ ID NO: 3 - DNA, 30 bp, Artificial Sequence
  rs3935591 primer
acgttggatgactgaggtccaggttccttg

SEQ ID NO: 4 - DNA, 30 bp, Artificial Sequence
  rs3935591 primer
acgttggatgtggctttcgtggaggaacag

SEQ ID NO: 5 - DNA, 30 bp, Artificial Sequence
  rs4778232 primer
acgttggatgaacagtttcttgcccatgcc

SEQ ID NO: 6 - DNA, 30 bp, Artificial Sequence
  rs4778232 primer
acgttggatgaagaaccaagggatctaggg

SEQ ID NO: 7 - DNA, 30 bp, Artificial Sequence
  rs8041209 primer
acgttggatgagaacttggtggaggatagc

SEQ ID NO: 8 - DNA, 29 bp, Artificial Sequence
  rs8041209 primer
acgttggatgtcttagagacaaaattccc

SEQ ID NO: 9 - DNA, 30 bp, Artificial Sequence
  rs1667394 primer
acgttggatgccattaagacgcagcaattc

SEQ ID NO: 10 - DNA, 31 bp, Artificial Sequence
  rs1667394 primer
acgttggatggtctttttctcctttcagttc

SEQ ID NO: 11 - DNA, 30 bp, Artificial Sequence
  rs16950987 primer
acgttggatgaattacccagcatgcatgac

SEQ ID NO: 12 - DNA, 30 bp, Artificial Sequence
  rs16950987 primer
acgttggatgcttgttactttatcttcctc

SEQ ID NO: 13 - DNA, 30 bp, Artificial Sequence
  rs2346050 primer
acgttggatggagcccagctgatttttctc

SEQ ID NO: 14 - DNA, 29 bp, Artificial Sequence
  rs2346050 primer
acgttggatgggaattcttccacttaatg

SEQ ID NO: 15 - DNA, 30 bp, Artificial Sequence
  rs1800407 primer
acgttggatgactctggcttgtactctctc

SEQ ID NO: 16 - DNA, 30 bp, Artificial Sequence
  rs1800407 primer
acgttggatgatgatgatcatggcccacac

SEQ ID NO: 17 - DNA, 30 bp, Artificial Sequence
  rs1129038 primer
acgttggatgcttctcatcagacacaccag

SEQ ID NO: 18 - DNA, 30 bp, Artificial Sequence
  rs1129038 primer
acgttggatgtcgtgagatgagagcctgag

SEQ ID NO: 19 - DNA, 30 bp, Artificial Sequence
  rs728405 primer
acgttggatgacccccatggaagaatgagc

SEQ ID NO: 20 - DNA, 30 bp, Artificial Sequence
  rs728405 primer
acgttggatgacataggatgcgtgagtgtg

SEQ ID NO: 21 - DNA, 30 bp, Artificial Sequence
  rs2240202 primer
acgttggatgtggcctcttacaggacttag

SEQ ID NO: 22 - DNA, 30 bp, Artificial Sequence
  rs2240202 primer
acgttggatgagtcctttaagcccggctac

SEQ ID NO: 23 - DNA, 29 bp, Artificial Sequence
  rs12592730 primer
acgttggatgagacagaaaagctgccaag

SEQ ID NO: 24 - DNA, 30 bp, Artificial Sequence
  rs12592730 primer
acgttggatgattctgctgttattggctgg

SEQ ID NO: 25 - DNA, 30 bp, Artificial Sequence
  rs7179994 primer
acgttggatgggctctaaccatagcatctc

SEQ ID NO: 26 - DNA, 30 bp, Artificial Sequence
  rs7179994 primer
acgttggatgccaacaaccacacagatgag

SEQ ID NO: 27 - DNA, 29 bp, Artificial Sequence
  rs7495174 primer
acgttggatgtaggtcggctccgtcgcac

SEQ ID NO: 28 - DNA, 30 bp, Artificial Sequence
  rs7495174 primer
acgttggatgggcttaggaagcaaggcaag

SEQ ID NO: 29 - DNA, 30 bp, Artificial Sequence
  rs1448485 primer
acgttggatgagcttcagcaagagcctaac

SEQ ID NO: 30 - DNA, 30 bp, Artificial Sequence
  rs1448485 primer
acgttggatgccccaccatattattaccag

SEQ ID NO: 31 - DNA, 30 bp, Artificial Sequence
  rs7183877 primer
acgttggatgctgtctcatgggtagtaatc

SEQ ID NO: 32 - DNA, 29 bp, Artificial Sequence
  rs7183877 primer
acgttggatgacacttgaagcagtataca

SEQ ID NO: 33 - DNA, 29 bp, Artificial Sequence
  rs683 primer
acgttggatgccttctttctaatacaagc

SEQ ID NO: 34 - DNA, 30 bp, Artificial Sequence
  rs683
acgttggatgttctgaaagggtcttcccag

SEQ ID NO: 35 - DNA, 30 bp, Artificial Sequence
  rs8028689 primer
acgttggatgttgtgctgctactcatctcc

SEQ ID NO: 36 - DNA, 30 bp, Artificial Sequence
  rs8028689 primer
acgttggatgagtgctagcaatgctaggtc

SEQ ID NO: 37 - DNA, 30 bp, Artificial Sequence
  rs12593929 primer
acgttggatgaggacacctgccaggactac

SEQ ID NO: 38 - DNA, 30 bp, Artificial Sequence
  rs12593929 primer
acgttggatggaagcacctgagagtgtctg

SEQ ID NO: 39 - DNA, 30 bp, Artificial Sequence
  rs16891982 primer
acgttggatgtctacgaaagaggagtcgag

SEQ ID NO: 40 - DNA, 30 bp, Artificial Sequence
  rs16891982 primer
acgttggatgaaagtgaggaaaacacggag

SEQ ID NO: 41 - DNA, 30 bp, Artificial Sequence
  rs4778138 primer
acgttggatgcctcccatcactgatttagc

SEQ ID NO: 42 - DNA, 31 bp, Artificial Sequence
  rs4778138 primer
acgttggatggaaagtctcaagggaaatcag

SEQ ID NO: 43 - DNA, 30 bp, Artificial Sequence
  rs12896399 primer
acgttggatggatgaggaaggttaatctgc

SEQ ID NO: 44 - DNA, 30 bp, Artificial Sequence
  rs12896399 primer
acgttggatgtctggcgatccaattctttg

SEQ ID NO: 45 - DNA, 30 bp, Artificial Sequence
  rs4778241 primer
acgttggatgaggagtgcaattgttggctg

SEQ ID NO: 46 - DNA, 30 bp, Artificial Sequence
  rs4778241 primer
acgttggatgtgtacagccactctggaaag

SEQ ID NO: 47 - DNA, 30 bp, Artificial Sequence
  rs916977 primer
acgttggatgttctgttcttcttgaccccg

SEQ ID NO: 48 - DNA, 30 bp, Artificial Sequence
  rs916977 primer
acgttggatgggtgtgggatttgttttggc

SEQ ID NO: 49 - DNA, 30 bp, Artificial Sequence
  rs12913832 primer
acgttggatgcgaggccagtttcatttgag

SEQ ID NO: 50 - DNA, 30 bp, Artificial Sequence
  rs12913832 primer
acgttggatgaaaacaaagagaagcctcgg

SEQ ID NO: 51 - DNA, 30 bp, Artificial Sequence
  rs8024968 primer
acgttggatgcagggagagtacagattcac

SEQ ID NO: 52 - DNA, 30 bp, Artificial Sequence
  rs8024968 primer
acgttggatgttggtgccttagatggactg

SEQ ID NO: 53 - DNA, 30 bp, Artificial Sequence
  rs16950979 primer
acgttggatggctctgctgctcttcttcca

SEQ ID NO: 54 - DNA, 30 bp, Artificial Sequence
  rs16950979 primer
acgttggatgaggaagcagacgataaggag

SEQ ID NO: 55 - DNA, 29 bp, Artificial Sequence
  rs2240203 primer
acgttggatgtctatattagcctcatcag

SEQ ID NO: 56 - DNA, 30 bp, Artificial Sequence
  rs2240203 primer
acgttggatggaagatcttgcttccaaagg

SEQ ID NO: 57 - DNA, 30 bp, Artificial Sequence
  rs2594935 primer
acgttggatggccacacaacttggatcttc

SEQ ID NO: 58 - DNA, 30 bp, Artificial Sequence
  rs2594935 primer
acgttggatgccacaggaaaacctgcaatg

SEQ ID NO: 59 - DNA, 30 bp, Artificial Sequence
  rs1597196 primer
acgttggatgaactctccgtgccttcctcc

SEQ ID NO: 60 - DNA, 30 bp, Artificial Sequence
  rs1597196 primer
acgttggatggcatgagttcacgtgtatga

SEQ ID NO: 61 - DNA, 30 bp, Artificial Sequence
  rs1393350 primer
acgttggatgggaaggtgaatgataacacg

SEQ ID NO: 62 - DNA, 30 bp, Artificial Sequence
  rs1393350 primer
acgttggatgtactcttcctcagtcccttc

SEQ ID NO: 63 - DNA, 30 bp, Artificial Sequence
  rs26722 primer
acgttggatggatggaatgtacgagtatgg

SEQ ID NO: 64 - DNA, 30 bp, Artificial Sequence
  rs26722 primer
acgttggatgtttttgctccctgcattgcc

SEQ ID NO: 65 - DNA, 30 bp, Artificial Sequence
  rs7170852 primer
acgttggatgatttgtagcagctgtgcgtc

SEQ ID NO: 66 - DNA, 30 bp, Artificial Sequence
  rs7170852 primer
acgttggatgaccaggccttctctttcatc

SEQ ID NO: 67 - DNA, 30 bp, Artificial Sequence
  rs1635168 primer
acgttggatgaatctcagagatcttacccg

SEQ ID NO: 68 - DNA, 30 bp, Artificial Sequence
  rs1635168 primer
acgttggatgactttgcctgagcacacaag

SEQ ID NO: 69 - DNA, 17 bp, Artificial Sequence
  rs3794604 extension primer
gctttgtggcctctcac

SEQ ID NO: 70 - DNA, 18 bp, Artificial Sequence
  rs3935591 extension primer
tccttgctggctgagcta

SEQ ID NO: 71 - DNA, 19 bp, Artificial Sequence
  rs4778232 extension primer
ctgccctcttcttcaacag

SEQ ID NO: 72 - DNA, 19 bp, Artificial Sequence
  rs8041209 extension primer
tggaggatagcctacagat

SEQ ID NO: 73 - DNA, 20 bp, Artificial Sequence
  rs1667394 extension primer
agcaattcaaaacgtgcata

SEQ ID NO: 74 - DNA, 20 bp, Artificial Sequence
  rs16950987 extension primer
tagcatgcatgactcatgaa

SEQ ID NO: 75 - DNA, 20 bp, Artificial Sequence
  rs2346050 extension primer
ttgatgacttagggttggtg

SEQ ID NO: 76 - DNA, 21 bp, Artificial Sequence
  rs1800407 extension primer
cccaggcataccggctctccc

SEQ ID NO: 77 - DNA, 22 bp, Artificial Sequence
  rs1129038 extension primer
ctacagtctacacagcagcgag

SEQ ID NO: 78 - DNA, 22 bp, Artificial Sequence
  rs728405 extension primer
gggaagaatgagccaaaaaaaa

SEQ ID NO: 79 - DNA, 24 bp, Artificial Sequence
  rs2240202 extension primer
actcttacaggacttagtaaccgc

SEQ ID NO: 80 - DNA, 24 bp, Artificial Sequence
  rs12592730 extension primer
tactggatccaatcaaaatttaca

SEQ ID NO: 81 - DNA, 24 bp, Artificial Sequence
  rs7179994 extension primer
gaagggttcagctggagcaaggtc

SEQ ID NO: 82 - DNA, 26 bp, Artificial Sequence
  rs7495174 extension primer
atccgtcgcacccgtctgtgcacact

SEQ ID NO: 83 - DNA, 26 bp, Artificial Sequence
  rs1448485 extension primer
ccatggttgttattaatactcatcaa

SEQ ID NO: 84 - DNA, 26 bp, Artificial Sequence
  rs7183877 extension primer
ggtagtaatcaaagaaacgacaagta

SEQ ID NO: 85 - DNA, 27 bp, Artificial Sequence
  rs683 extension primer
cttctttctaatacaagcatatgttag

SEQ ID NO: 86 - DNA, 17 bp, Artificial Sequence
  rs8028689 extension primer
ctcagtgttccacttcc

SEQ ID NO: 87 - DNA, 18 bp, Artificial Sequence
  rs12593929 extension primer
gggcccacctgccacacg

SEQ ID NO: 88 - DNA, 18 bp, Artificial Sequence
  rs16891982 extension primer
ggttggatgttggggctt

SEQ ID NO: 89 - DNA, 19 bp, Artificial Sequence
  rs4778138 extension primer
ctgatttagctgtgttctg

SEQ ID NO: 90 - DNA, 20 bp, Artificial Sequence
  rs12896399 extension primer
tgtctgctgtgacaaagaga

SEQ ID NO: 91 - DNA, 20 bp, Artificial Sequence
  rs4778241 extension primer
aggggctggtagttgcaatt

SEQ ID NO: 92 - DNA, 21 bp, Artificial Sequence
  rs916977 extension primer
ttcagccttggccagccttct

SEQ ID NO: 93 - DNA, 21 bp, Artificial Sequence
  rs12913832 extension primer
ccagtttcatttgagcattaa

SEQ ID NO: 94 - DNA, 22 bp, Artificial Sequence
  rs8024968 extension primer
gagagtacagattcacagactt

SEQ ID NO: 95 - DNA, 23 bp, Artificial Sequence
  rs16950979 extension primer
gtttactcttcttccagctcttc

SEQ ID NO: 96 - DNA, 23 bp, Artificial Sequence
  rs2240203 extension primer
tgtcttaatgtttacattcctta

SEQ ID NO: 97 - DNA, 23 bp, Artificial Sequence
  rs2594935 extension primer
tggatcttcttgtagcaagtaac

SEQ ID NO: 98 - DNA, 24 bp, Artificial Sequence
  rs1597196 extension primer
cccaggctctggaacctgcaattt

SEQ ID NO: 99 - DNA, 24 bp, Artificial Sequence
  rs1393350 extension primer
ggtggtaaaagaccacacagattt

SEQ ID NO: 100 - DNA, 25 bp, Artificial Sequence
  rs26722 extension primer
gggagtgtacgagtatggttctatc

SEQ ID NO: 101 - DNA, 26 bp, Artificial Sequence
  rs7170852 extension primer
tttgtagcagctgtgcgtctgtttcc

SEQ ID NO: 102 - DNA, 27 bp, Artificial Sequence
  rs1635168 extension primer
cctcccagagatcttacccgtacctga

SEQ ID NO: 103 - DNA, 20 bp, Artificial Sequence
  rs12913832 forward primer
tggctctctgtgtctgatcc

SEQ ID NO: 104 - DNA, 19 bp, Artificial Sequence
  rs12913832 reverse primer
ggcccctgatgatgatagc

SEQ ID NO: 105 - DNA, 20 bp, Artificial Sequence
  rs1800407 forward primer
tgaaaggctgcctctgttct

SEQ ID NO: 106 - DNA, 21 bp, Artificial Sequence
  rs1800407 reverse primer
cgatgagacagagcatgatga

SEQ ID NO: 107 - DNA, 20 bp, Artificial Sequence
  rs12896399 forward primer
ctggcgatccaattctttgt

SEQ ID NO: 108 - DNA, 20 bp, Artificial Sequence
  rs12896399 reverse primer
cttagccctgggtcttgatg

SEQ ID NO: 109 - DNA, 21 bp, Artificial Sequence
  rs16891982 forward primer
tccaagttgtgctagaccaga

SEQ ID NO: 110 - DNA, 20 bp, Artificial Sequence
  rs16891982 reverse primer
cgaaagaggagtcgaggttg

SEQ ID NO: 111 - DNA, 20 bp, Artificial Sequence
  rs1393350 forward primer
ttcctcagtcccttctctgc

SEQ ID NO: 112 - DNA, 21 bp, Artificial Sequence
  rs1393350 reverse primer
gggaaggtgaatgataacacg

SEQ ID NO: 113 - DNA, 20 bp, Artificial Sequence
  rs12203592 forward primer
acagggcagctgatctcttc

SEQ ID NO: 114 - DNA, 20 bp, Artificial Sequence
  rs12203592 reverse primer
gctaaacctggcaccaaaag

SEQ ID NO: 115 - DNA, 41 bp, Artificial Sequence
  rs12913832 extension primer
ttttttttttttttttttttttttgcgtgcagaacttgaca

SEQ ID NO: 116 - DNA, 23 bp, Artificial Sequence
  rs1800407 extension primer
tttttttttcccacacccgtccc

SEQ ID NO: 117 - DNA, 53 bp, Artificial Sequence
  rs12896399 extension primer
tttttttttttttttttttttttttttttatctttaggtcagtatattttggg

SEQ ID NO: 118 - DNA, 29 bp, Artificial Sequence
  rs16891982 extension primer
tttttttttttaaacacggagttgatgca

SEQ ID NO: 119 - DNA, 47 bp, Artificial Sequence
  rs1393350 extension primer
tttttttttttttttttttttttatttgtaaaagaccacacagattt

SEQ ID NO: 120 - DNA, 35 bp, Artificial Sequence
  rs12203592 extension primer
tttttttttttttttaaagtaccacaggggaattt

SEQ ID NO: 121 - DNA, 23 bp, Artificial Sequence
  rs1800407 extension primer
tttttttgcataccggctctccc

SEQ ID NO: 122 - DNA, 35 bp, Artificial Sequence
  rs12203592 extension primer
tttttttttttttttatttggtgggtaaaagaagg

(19) United States
(12) Patent Application Publication
(10) Pub. No.: US 2011/0312534 A1
(43) Pub. Date: Dec. 22, 2011

(54) METHOD FOR PREDICTION OF HUMAN IRIS COLOR

(75) Inventors: Manfred Heinz Kayser, Rotterdam (NL); Fan Liu, Rotterdam (NL); Albert Hofman, Rotterdam (NL)

(73) Assignee: Erasmus University Medical Center Rotterdam, Rotterdam (NL)

(21) Appl. No.: 13/041,109

(22) Filed: Mar. 4, 2011

Related U.S. Application Data
(60) Provisional application No. 61/311,238, filed on Mar. 5, 2010.

(51) Int. Cl. C40B 30/10; C40B 30/00; C07H 21/00; C12Q 1/68

(52) U.S. Cl. 506/12; 435/6.11; 506/7; 536/24.33

(57) ABSTRACT

A method for predicting the iris color of a human, the method comprising:
(a) obtaining a sample of the nucleic acid of the human;
(b) genotyping the nucleic acid for at least the following polymorphisms:
   (i) the single nucleotide polymorphism (SNP) rs12913832 or a polymorphic site which is in linkage disequilibrium with rs12913832 at an r² value of at least 0.9;
   (ii) the SNP rs1800407 or a polymorphic site which is in linkage disequilibrium with rs1800407 at an r² value of at least 0.5; and,
   (iii) the SNP rs12896399 or a polymorphic site which is in linkage disequilibrium with rs12896399 at an r² value of at least 0.5; and
(c) predicting the iris color based on the results of step (b).

A method of genotyping said polymorphisms, and kits comprising or a solid substrate having attached thereto nucleic acid molecules suitable for performing the method.

### FIELD OF THE INVENTION

The present invention relates to a method for prediction of the phenotype of a complex polygenic trait. In particular, it relates to a method for prediction of human iris color.

### BACKGROUND OF THE INVENTION

Predicting externally visible characteristics (EVCs) using informative molecular markers, such as those from DNA, has started to become a rapidly developing area in forensic genetics. With knowledge gleaned from this type of data, it could be viewed as a ‘biological witness’ tool in suitable forensic cases, leading to a new era of ‘DNA intelligence’ (sometimes referred to as Forensic DNA Phenotyping); an era in which the externally visible traits of an individual may be defined solely from a biological sample left at a crime scene or from a dismembered part of a missing person. Human eye (iris) color is a highly polymorphic phenotype in people of European descent and, albeit less so, in those from surrounding regions such as the Middle East or Western Asia, and is under strong genetic control (R. A. Sturm, T. N. Frudakis, Trends Genet. 20 (2004) 327-332). Most human populations around the world have non-variable dark brown iris color while blue, green, gray and light brown colors are additionally found in people of European descent, and people originating from Europe-neighbouring regions. Thus, the DNA-based prediction of iris color may be useful in identifying persons of European and neighboring descent, or persons residing in an area which is populated by persons of European descent.

Currently, human identification using nucleic acid markers is completely based on comparing marker profiles (DNA fingerprints, DNA profiles) obtained from crime scene samples with those obtained from known suspects. If no suspect (or close relative thereof) is known to the police no profile can be obtained and compared with the one collected from the crime scene. Consequently, in such cases the person who left the sample at the crime scene and who might have committed the crime can not be identified using genetic (DNA) evidence. Similarly, missing persons are currently identified by comparing a DNA profile obtained from their remains with that obtained from a known relative. If nothing is known about the missing person, no relatives can be identified for genetic testing and no DNA profile is available for comparison. The identification of nucleic acid markers that could reliably predict eye (iris) color would help in finding unknown persons (suspects/missing persons) in a direct way and without comparing DNA profiles.

Recent years have yielded intensive studies to increase the genetic understanding of human eye color, via genome-wide association and linkage analysis or candidate gene studies (Sulem et al, Nat. Genet. 39 (2007) 1443-1452; Eiberg et al, Hum. Genet. 123 (2008) 177-187; Kayser et al, Am. J. Hum. Genet. 82 (2008) 411-423; Sturm et al, Am. J. Hum. Genet. 82 (2008) 424-431; Han et al, PLoS Genet. 4 (2008) e1000074; Sulem et al, Nat. Genet. 40 (2008) 835-837; Kanetsky et al, Am. J. Hum. Genet. 70 (2002) 770-775; Duffy et al, Am. J. Hum. Genet. 80 (2007) 241-252; Zhu et al, Twin Res. 7 (2004) 197-210; Posthuma et al, Behav. Genet. 36 (2006) 12-17; Frudakis et al, Genetics 165 (2003) 2071-2083). The OCA2 gene on chromosome 15 was originally thought to be the most informative human eye color gene due to its association with the human P protein required for the processing of melanosomal proteins, and mutations in this gene do result in pigmentation disorders. However, recent studies have shown that genetic variants in the neighbouring HERC2 gene are more significantly associated with eye color variation than those in OCA2 (Sulem et al, 2007, supra; Eiberg et al, supra; Kayser et al, supra; Sturm et al, supra; Han et al, supra). Also, one of the most significant non-synonymous SNPs associated with eye color, rs1800407 located in exon 12 of the OCA2 gene, acts only as a penetrance modifier of rs12913832 in HERC2 and is, to a lesser extent, independently associated with eye color variation (Sturm et al, supra). While the HERC2/OCA2 region harbours most blue and brown eye color information, other genes were also identified as contributing to eye color variation, such as SLC24A4, SLC45A2 (MATP), TYRP1, TYR, ASIP, IRF4, CYP1A2, CYP2C8, and CYP2C9 although to a much lesser degree (Sulem et al 2007, supra; Han et al, supra; Sulem et al 2008, supra; Kanetsky et al, supra; Frudakis et al, supra; WO 2002/097047).

Despite this abundance of information concerning the association of various polymorphisms with human iris color variation, there have been few attempts to predict iris color of an individual based on their genotype. Sulem et al, 2007, supra, attempted to predict iris color using polymorphisms within various genes and concluded that, in their study, prediction of blue versus brown iris color is dominated by variants in OCA2. However, in WO 2009/025544 (Kayser et al; Erasmus University Medical Center Rotterdam) and the corresponding publication Kayser et al. 2008, supra, various SNPs within the HERC2 gene were found to be more useful than variations within OCA2 for prediction of iris color. Identifying the most useful polymorphisms for prediction is not simply a matter of using the polymorphisms which are most strongly associated with iris color variation. The P-values derived from the association testing do not provide sufficient information on the prediction accuracy of the SNPs involved. Further, the genetic association analyses were mostly based on iteratively testing the association between a single SNP and eye color. This does not consider various combinations of associated SNPs, which is important when SNPs are not independent of each other, e.g. in linkage disequilibrium or in genetic interaction. Rather, identifying the most useful polymorphisms for prediction requires analysis of a combination of informative SNPs and application of a dedicated prediction methodology.

Neither is it practical to generate a prediction model using all known polymorphisms, as this would require large numbers of polymorphisms to be genotyped every time that the model was to be applied in order to arrive at a prediction, which would be costly and laborious.

There is therefore a need for a more accurate and yet simple genetic test for prediction of iris color.

The listing or discussion of an apparently prior-published document in this specification should not necessarily be taken as an acknowledgement that the document is part of the state of the art or is common general knowledge.

### SUMMARY OF THE INVENTION

A first aspect of the invention provides a method for predicting the iris color of a human, the method comprising:
(a) obtaining a sample of the nucleic acid of the human;
(b) genotyping the nucleic acid for at least the following polymorphisms:
   (i) the single nucleotide polymorphism (SNP) rs12913832 or a polymorphic site which is in linkage disequilibrium with rs12913832 at an r² value of at least 0.9;
   (ii) the SNP rs1800407 or a polymorphic site which is in linkage disequilibrium with rs1800407 at an r² value of at least 0.5; and,
   (iii) the SNP rs12896399 or a polymorphic site which is in linkage disequilibrium with rs12896399 at an r² value of at least 0.5; and
(c) predicting the iris color based on the results of step (b).

A second aspect of the invention provides a method of preparing a data carrier containing data on the predicted iris color of a human, the method comprising recording the results of a method carried out according to the first aspect of the invention on a data carrier.

A third aspect of the invention provides a method for predicting the iris color of a human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

A fourth aspect of the invention provides a method for creating a description of a human based on forensic testing, wherein the description includes a prediction of the iris color of the human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

A fifth aspect of the invention provides a method for genotyping polymorphisms indicative of human iris color comprising:
(a) obtaining a sample of the nucleic acid of a human; and
(b) genotyping the nucleic acid for at least the following polymorphisms:
   (i) the single nucleotide polymorphism (SNP) rs12913832 or a polymorphic site which is in linkage disequilibrium with rs12913832 at an r² value of at least 0.9;
   (ii) the SNP rs1800407 or a polymorphic site which is in linkage disequilibrium with rs1800407 at an r² value of at least 0.5; and,
   (iii) the SNP rs12896399 or a polymorphic site which is in linkage disequilibrium with rs12896399 at an r² value of at least 0.5.

A sixth aspect of the invention provide a kit of parts for use in predicting the iris color of a human comprising:

(i) a primer pair suitable for amplifying the genomic region encompassing the single nucleotide polymorphism (SNP) rs12913832 or a polymorphic site which is in linkage disequilibrium with rs12913832 at an r² value of at least 0.9;
(ii) a primer pair suitable for amplifying the genomic region encompassing the SNP rs1800407 or a polymorphic site which is in linkage disequilibrium with rs1800407 at an r² value of at least 0.5; and,
(iii) a primer pair suitable for amplifying the genomic region encompassing the SNP rs12896399 or a polymorphic site which is in linkage disequilibrium with rs12896399 at an r² value of at least 0.5.

A seventh aspect of the invention provides a kit of parts for use in predicting the iris color of a human comprising:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

An eighth aspect of the invention provides a solid substrate for use in predicting the iris color of a human, the solid substrate having attached thereto:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

### DESCRIPTION OF FIGURES

FIG. 1. Contribution of 24 SNPs to the Prediction Accuracy of Human Eye Color in Dutch Europeans

Prediction performance measured by AUC for the model based on multinomial logistic regression (Y-axis) was plotted against the number of SNPs included in the model (X-axis). For each step, the lowest contributor in the model-building set (N=3804) was excluded from the model; the model was rebuilt and used to predict eye color in the model-verification set (N=2364). The prediction of blue is represented by squares; brown is represented by triangles; and intermediate is represented by diamonds.

FIG. 2. ROC curve of Dutch European cohort (n=2364) prepared from previously published data [Example 1]. True positive rates on y-axis were plotted against all false positive rate thresholds on x-axis. The greatest AUC is for the brown prediction (squares); the second AUC is for the blue prediction (circles) and the lowest AUC is for the intermediate prediction (stars).

FIG. 3. Hypothesised scenario for genetic determination of brown and blue eye colors showing the impact of the most influential SNP genotypes from the 6-SNP model.

FIG. 4. Worldwide genotype distribution of the 6 IrisPlex™ SNPs in 934 individuals of the H952 HGDP-CEPH set from 51 worldwide population groups, in order of prediction rank revealed from a large Dutch cohort [10]: (a) rs12913832 (HERC2), (b) rs1800407 (OCA2), (c) rs12896399 (SLC24A4), (d) rs16891982 (SLC45A2(MATP)), (e) rs1393350 (TYR), (f) rs12203592 (IRF4). White indicates the proportion of individuals with blue-eye-associated homozygote genotypes as revealed from previous European studies, black indicates the proportion of individuals with brown-eye-associated homozygote genotypes from previous European studies, and hatched indicates the proportion of individuals with heterozygote genotypes.

FIG. 5. IrisPlex™ eye color prediction on a worldwide scale, using 934 individuals of the H952 HGDP-CEPH set from 51 worldwide populations and applying a prediction probability threshold of 0.7. White indicates the proportion of individuals with predicted blue eye color, hatched indicates the proportion of individuals with predicted brown eye color, and black indicates undefined individuals given the prediction probability threshold applied.

FIG. 6. Non-metric multidimensional scaling (MDS) plot of the pairwise F_{ST} distances between HGDP-CEPH populations using the 6 IrisPlex™ SNPs, color code is according to geographic regions as provided in the legend. All populations with variation in IrisPlex™ predicted eye color are given with names.

### DETAILED DESCRIPTION OF PREFERRED EMBODIMENTS OF THE INVENTION

A first aspect of the invention provides a method for predicting the iris color of a human, the method comprising:
(a) obtaining a sample of the nucleic acid of the human;
(b) genotyping the nucleic acid for at least the following polymorphisms:
   (i) the single nucleotide polymorphism (SNP) rs12913832 or a polymorphic site which is in linkage disequilibrium with rs12913832 at an r² value of at least 0.9;
   (ii) the SNP rs1800407 or a polymorphic site which is in linkage disequilibrium with rs1800407 at an r² value of at least 0.5; and,
   (iii) the SNP rs12896399 or a polymorphic site which is in linkage disequilibrium with rs12896399 at an r² value of at least 0.5; and
(c) predicting the iris color based on the results of step (b).

The sample of nucleic acid from the human may be any suitable sample and includes genomic DNA, RNA and cDNA. Genomic DNA is preferred because most SNPs are in non-translated regions, but for the avoidance of doubt and where the context permits it, the term “sample” also includes cDNA derived from other nucleic acid in the sample and mRNA. The nucleic acid may be isolated from any raw sample material, optionally reverse transcribed into cDNA and directly cloned and/or sequenced. DNA and RNA isolation kits are commercially available from for instance QIAGEN GmbH, Hilden, Germany, or Roche Diagnostics, a division of F. Hoffmann-La Roche Ltd, Basel, Switzerland.

A sample useful for practicing a method of the invention can be any biological sample of a subject that contains nucleic acid molecules, including portions of the gene sequences to be examined. As such, the sample can be a cell, tissue or organ sample, or can be a sample of a biological fluid such as semen, saliva, blood, and the like.

In a forensic application of a method of the invention, the human nucleic acid sample can be obtained from a crime scene, using well established sampling methods. Thus, the sample can be a fluid sample or a swab sample for example blood stain, semen stain, hair follicle, or other biological specimen, taken from a crime scene, or can be a soil sample suspected of containing biological material of a potential crime victim or perpetrator, can be material retrieved from under the finger nails of a putative crime victim, or the like. Another application of the invention is in identifying missing persons (such as deceased persons or parts thereof but potentially also missing persons who are unable or unwilling for whatever reason to disclose their identity) by analysing the herein identified markers from nucleic acids from samples of the unknown person to be identified. A suitable sample may be obtained from a cell, tissue or organ sample, including bone material, or may be a biological fluid.

Another suitable application of the method is in preimplantation or prenatal diagnostics in which case the sample would be extracted from cellular material of the embryo or fetus.

The human from whom the nucleic acid sample is obtained can be of any race. As such, the human can be of any group of people classified together on the basis of common history, nationality, or geographic distribution. For example, the subject can be of African, Asian, such as West Asian, Australasian, European, Middle Eastern, North American or South American descent. In certain embodiments the human is Asian, Hispanic, African, or Caucasian. In one embodiment the human is Caucasian. In one embodiment the human is of European, West Asian or Middle Eastern descent, as iris color variation is generally confined to such persons. Often the race of the human subject may not be known. The term “of European descent” means an individual who is a descendant of an individual who was born in a European country or territory in the 11^{th} through 20^{th} centuries, typically in the 15^{th} through 18^{th} centuries. Typically, at least 10%, at least 15%, at least 20%, at least 25%, 30%, 40%, 50%, 60%, 70%, 75%, 80%, 90% or 95% and up to 100% of the genetic material of a person of European descent is derived from ancestors who were born in a European country/territory or European countries/territories. The term “of West Asian descent” or “of Middle Eastern descent” can be understood accordingly.

European countries include the following: Albania, Andorra, Armenia, Austria, Azerbaijan, Belarus, Belgium, Bosnia and Herzegovina, Bulgaria, Croatia, Cyprus, Czech Republic, Denmark, Estonia, Finland, France, Georgia, Germany, Greece, Hungary, Iceland, Ireland, Italy, Kazakhstan, Latvia, Liechtenstein, Lithuania, Luxembourg, Macedonia, Malta, Moldova, Monaco, Montenegro, The Netherlands, Norway, Poland, Portugal, Romania, Russia, San Marino, Serbia, Slovakia, Slovenia, Spain, Sweden, Switzerland, Turkey, Ukraine, United Kingdom and Vatican City. European territories include the following: Aland, Akrotiri and Dhekelia, Faroe Islands, Gibraltar, Guernsey, Isle of Man, Jersey, Abkhazia, Kosovo, Northern Cyprus and South Ossetia. Middle Eastern countries include the following: Turkey, Bahrain, Kuwait, Oman, Qatar, Saudi Arabia, United Arab Emirates, Yemen, Gaza strip, Iraq, Israel, Jordan, Lebanon, Syria, West Bank, Iran, Cyprus and Egypt. West Asian countries include the following: Armenia, Azerbaijan, Bahrain, Cyprus, Georgia, Iraq, Israel, Jordan, Kuwait, Lebanon, Oman, Palestine, Pakistan, Qatar, Saudi Arabia, Syria, Turkey, United Arab Emirates and Yemen.

The SNP rs12913832 is in the HERC2 gene on chromosome 15, and the allele may be either A with reference to the positive DNA strand (or, when considering the complementary DNA strand, T) or G (or, when considering the complementary DNA strand, C). The G allele has been associated with blue iris color (Eiberg et al 2008 Hum Genet 123: 177-187). It is possible that T or C alleles, while referring to the same strand as A and G before, might also exist at this locus, although these have not been identified. The SNP rs1800407 is in the OCA2 gene on chromosome 15, and the allele may be either C or T with reference to the positive DNA strand (or, when considering the complementary DNA strand, G or A). Again, it is possible that other alleles might exist at this locus. The effect of the polymorphism is to change the amino acid sequence at position 419 of OCA2 protein, with Arg419Gln caused by C (or G)→T (or A) associated with non-blue eye color (Duffy et al 2007 Am J Hum Genet 80: 241-252). The SNP rs12896399 is in the SLC24A4 gene on chromosome 14 and the allele may be either G or T with reference to the positive DNA strand, although it is possible that other alleles might exist at this locus. The T allele has been associated with blue versus green eyes (Sulem et al 2007 Nature Genetics 39: 1443-1452). The inventors have found that these three markers are the most useful markers for prediction of human iris color. Individuals may be either homozygous or heterozygous for a given allele of any of these SNPs.

The prediction of iris color involves analyzing the nucleotide occurrences of each of these SNPs (or polymorphisms having the required degree of linkage disequilibrium with the SNPs) in a nucleic acid sample of the subject, and comparing the combination of nucleotide occurrences of the SNPs (or genotypes of the linked polymorphisms) to known relationships of genotype and iris color. Thus, the iris color may be inferred from the genotypes of the polymorphisms that have been analyzed.

Typically, the polymorphic sites are SNPs; however, they may be an insertion, a deletion, a microsatellite or an inversion or a combination of these. The polymorphic sites disclosed herein may or may not be causative. Polymorphic sites which are in linkage disequilibrium with rs12913832, rs1800407 or rs12896399 may be used as proxy markers. If two loci are in linkage disequilibrium (LD), it means that the degree of recombination between these loci within a population is low. In other words, particular alleles tend to be inherited together. In that case, the presence of an allele at one locus may be predictive of the presence of a particular allele at the other locus, such that one can be used as a proxy for the other. The degree of linkage disequilibrium (LD) between two markers is typically indicated by the parameter r², with an r² value of 1 indicating complete LD and an r² value of 0 indicating complete independence. The extent of LD between markers can vary to an extent depending on the population. As iris color variation is most prevalent among Europeans, a European population is the most relevant population for the determination of LD. Unless otherwise stated herein, r² values are given for European populations.

If a polymorphic site which is in linkage disequilibrium with rs12913832 is to be used, it should be in high linkage disequilibrium because rs12913832 contributes most substantially to the predictive accuracy of the method, and polymorphisms with a relatively low linkage disequilibrium with rs12913832 would reduce the predictive accuracy of the method. A suitable polymorphic site which may be used in place of rs12913832 is one which is in linkage disequilibrium with rs12913832 at an r² value of at least 0.9, preferably at least 0.95, more preferably at least 0.975, or at least 0.99. rs1129038 (26030454bp on chromosome 15) is a known SNP which is in linkage disequilibrium with rs12913832 at an r² value of at least 0.9; the relevant r² value is 0.99.

Rs1800407A contributes less to the predictive accuracy of the method compared to rs12913832. The method will still provide an adequate predictive accuracy if a polymorphic site having a lower degree of linkage disequilibrium is to be used in place of rs1800407. A suitable polymorphic site which may be used in place of rs1800407 is one which is in linkage disequilibrium with rs1800407 at an r² value of at least 0.5, suitably at least 0.6, at least 0.7, at least 0.8, at least 0.9 or at least 0.95. SNPs having the required linkage disequilibrium with rs1800407, or other SNPs useful in the invention are listed in Table 1. SNP positions and chromosomal locations indicated throughout this document are according to NCBI Build 36.

TABLE 1
SNPs having the required linkage disequilibrium with SNPs useful in the invention

| | | | | | | | |
|---|---|---|---|---|---|---|---|
| SNP | Position | LD(r²) | DataSource | SNP | Position | LD(r²) | DataSource |

rs16891982 SLC45A2

| | | | | | | | |
|---|---|---|---|---|---|---|---|
| rs35407 | 33982328 | 0.772 | RS | rs35389 | 33990637 | 0.896 | RS |
| rs35395 | 33984346 | 0.784 | RS | rs28777 | 33994716 | 0.896 | RS |
| rs35397 | 33986873 | 0.682 | RS | rs183671 | 33999967 | 0.896 | RS |
| rs2278007 | 33987308 | 0.889 | RS | rs3797201 | 34003902 | 0.883 | RS |

rs1393350 TYR

| | | | | | | | |
|---|---|---|---|---|---|---|---|
| rs10765198 | 88609422 | 0.862 | RS | rs11018464 | 88460762 | 0.52 | HapMapCEU |
| rs7358418 | 88609786 | 0.862 | RS | rs12363323 | 88495940 | 0.535 | HapMapCEU |
| rs10765200 | 88611332 | 0.862 | RS | rs1942486 | 88496430 | 0.52 | HapMapCEU |
| rs10765201 | 88611352 | 0.862 | RS | rs17792911 | 88502470 | 0.53 | HapMapCEU |
| rs4396293 | 88615761 | 0.522 | RS | rs10830219 | 88512157 | 0.535 | HapMapCEU |
| rs2186640 | 88615811 | 0.531 | RS | rs10830236 | 88540464 | 0.597 | HapMapCEU |
| rs10501698 | 88617012 | 0.797 | RS | rs12270717 | 88551838 | 0.872 | HapMapCEU |
| rs10830250 | 88617255 | 0.558 | RS | rs7129973 | 88555218 | 0.514 | HapMapCEU |
| rs7924589 | 88617956 | 0.697 | RS | rs11018525 | 88559553 | 0.514 | HapMapCEU |
| rs4121401 | 88619494 | 0.639 | RS | rs17793678 | 88561172 | 1 | HapMapCEU |
| rs1847134 | 88644901 | 0.791 | RS | rs10765196 | 88564890 | 1 | HapMapCEU |
| rs1827430 | 88658088 | 0.57 | RS | rs10765197 | 88564976 | 0.514 | HapMapCEU |
| rs3900053 | 88660713 | 0.758 | RS | rs7123654 | 88565603 | 0.512 | HapMapCEU |
| rs1847142 | 88661222 | 0.808 | RS | rs11018528 | 88570025 | 1 | HapMapCEU |
| rs4121403 | 88664103 | 0.694 | RS | rs12791412 | 88570229 | 0.936 | HapMapCEU |
| rs10830253 | 88667691 | 0.807 | RS | rs12789914 | 88570555 | 0.761 | HapMapCEU |
| rs7951935 | 88670047 | 0.619 | RS | rs7107143 | 88571135 | 0.827 | HapMapCEU |
| rs1847140 | 88676712 | 0.684 | RS | rs4512823 | 88606232 | 0.87 | HapMapCEU |
| rs1806319 | 88677584 | 0.634 | RS | rs4512825 | 88606499 | 0.777 | HapMapCEU |
| rs4106039 | 88680791 | 0.568 | RS | rs7101897 | 88647570 | 0.779 | HapMapCEU |
| rs4106040 | 88680802 | 0.608 | RS | rs1126809 | 88657609 | 0.827 | HapMapCEU |
| rs11018463 | 88459390 | 0.535 | HapMapCEU | | | | |

rs12896399 SLC24A4

| | | | | | | | |
|---|---|---|---|---|---|---|---|
| rs8017054 | 91830169 | 0.651 | RS | rs1885194 | 91847215 | 0.992 | RS |
| rs4900109 | 91833144 | 0.985 | RS | rs17184180 | 91850140 | 0.992 | RS |
| rs4904866 | 91838256 | 1 | RS | rs4904868 | 91850754 | 0.661 | RS |
| rs746586 | 91845720 | 1 | RS | rs4904870 | 91856761 | 0.661 | RS |
| rs1075830 | 91845915 | 0.661 | RS | rs4900114 | 91865488 | 0.653 | HapMapCEU |
| rs941799 | 91846578 | 0.992 | RS | | | | |

rs12913832 HERC2

| | | | |
|---|---|---|---|
| rs1129038 | 26030454 | 0.99 | RS |

rs12203592 IRF4

None
identified

rs1408799 TYRP1

| | | | | | | | |
|---|---|---|---|---|---|---|---|
| rs13283649 | 12608337 | 0.663 | RS | rs2762461 | 12686499 | 0.674 | RS |
| rs7466934 | 12609840 | 0.665 | RS | rs2733831 | 12693484 | 0.622 | RS |
| rs7036899 | 12610266 | 0.666 | RS | rs2733832 | 12694725 | 0.657 | RS |
| rs10756386 | 12611004 | 0.666 | RS | rs10960758 | 12706315 | 0.725 | RS |
| rs10960723 | 12612878 | 0.621 | RS | rs10960759 | 12706428 | 0.725 | RS |
| rs977888 | 12614357 | 0.666 | RS | rs12379024 | 12707405 | 0.725 | RS |
| rs10809808 | 12614463 | 0.621 | RS | rs13295868 | 12707912 | 0.725 | RS |
| rs10960730 | 12621099 | 0.623 | RS | rs7019226 | 12708370 | 0.707 | RS |
| rs10809809 | 12621398 | 0.623 | RS | rs11789751 | 12709264 | 0.725 | RS |
| rs10960732 | 12623495 | 0.623 | RS | rs10491744 | 12710106 | 0.725 | RS |
| rs7026116 | 12623981 | 0.623 | RS | rs10960760 | 12710152 | 0.725 | RS |
| rs7047297 | 12628540 | 0.644 | RS | rs2382361 | 12710786 | 0.725 | RS |
| rs10960735 | 12631821 | 0.695 | RS | rs1409626 | 12710820 | 0.725 | RS |
| rs1325122 | 12632878 | 0.647 | RS | rs1409630 | 12711251 | 0.705 | RS |
| rs10809811 | 12640996 | 0.695 | RS | rs13288475 | 12711714 | 0.705 | RS |
| rs1408794 | 12641340 | 0.695 | RS | rs13288636 | 12711806 | 0.705 | RS |
| rs13294940 | 12642364 | 0.664 | RS | rs13288681 | 12711881 | 0.705 | RS |
| rs995263 | 12644578 | 0.648 | RS | rs1326798 | 12712227 | 0.705 | RS |
| rs1121541 | 12657049 | 0.696 | RS | rs12379260 | 12713112 | 0.705 | RS |
| rs10809818 | 12658121 | 0.53 | RS | rs13284453 | 12714280 | 0.645 | RS |
| rs1325127 | 12658328 | 0.53 | RS | rs13284898 | 12714560 | 0.705 | RS |
| rs10960748 | 12658805 | 0.76 | RS | rs10960774 | 12729313 | 0.595 | RS |
| rs9298679 | 12659346 | 0.615 | RS | rs10756406 | 12738587 | 0.607 | RS |
| rs10960749 | 12661566 | 0.762 | RS | rs927868 | 12738795 | 0.577 | RS |
| rs1408800 | 12662275 | 1 | RS | rs927869 | 12738962 | 0.607 | RS |
| rs13294134 | 12663636 | 0.762 | RS | rs4741245 | 12739300 | 0.607 | RS |
| rs10960751 | 12665264 | 0.71 | RS | rs7023927 | 12739596 | 0.607 | RS |
| rs10960752 | 12665284 | 0.71 | RS | rs7035500 | 12740095 | 0.607 | RS |
| rs10960753 | 12665522 | 0.709 | RS | rs13302551 | 12740812 | 0.592 | RS |
| rs13296454 | 12667181 | 0.708 | RS | rs1543587 | 12741741 | 0.607 | RS |
| rs13297008 | 12667471 | 0.708 | RS | rs1074789 | 12742340 | 0.595 | RS |
| rs10809826 | 12672663 | 0.726 | RS | rs10960779 | 12748881 | 0.593 | RS |
| rs2762460 | 12686478 | 0.623 | RS | | | | |

rs683 TYRP1

| | | | | | | | |
|---|---|---|---|---|---|---|---|
| rs13283649 | 12608337 | 0.561 | RS | rs2224863 | 12692890 | 0.993 | RS |
| rs7466934 | 12609840 | 0.563 | RS | rs2733830 | 12693359 | 0.915 | RS |
| rs7036899 | 12610266 | 0.564 | RS | rs2733831 | 12693484 | 0.68 | RS |
| rs10756386 | 12611004 | 0.564 | RS | rs2733832 | 12694725 | 0.759 | RS |
| rs10960723 | 12612878 | 0.522 | RS | rs2733833 | 12695095 | 0.94 | RS |
| rs977888 | 12614357 | 0.564 | RS | rs2209277 | 12696236 | 0.915 | RS |
| rs10809808 | 12614463 | 0.522 | RS | rs10809828 | 12697861 | 0.582 | RS |
| rs10960730 | 12621099 | 0.523 | RS | rs2733834 | 12698910 | 0.92 | RS |
| rs10809809 | 12621398 | 0.523 | RS | rs2762464 | 12699586 | 0.973 | RS |
| rs10960732 | 12623495 | 0.523 | RS | rs910 | 12700035 | 0.893 | RS |
| rs7026116 | 12623981 | 0.523 | RS | rs1063380 | 12700090 | 0.893 | RS |
| rs7047297 | 12628540 | 0.538 | RS | rs10960758 | 12706315 | 0.66 | RS |
| rs13301970 | 12629877 | 0.761 | RS | rs10960759 | 12706428 | 0.66 | RS |
| rs10960735 | 12631821 | 0.587 | RS | rs12379024 | 12707405 | 0.66 | RS |
| rs1325122 | 12632878 | 0.541 | RS | rs13295868 | 12707912 | 0.66 | RS |
| rs10960738 | 12638831 | 0.807 | RS | rs7019226 | 12708370 | 0.684 | RS |
| rs13283345 | 12640198 | 0.807 | RS | rs11789751 | 12709264 | 0.66 | RS |
| rs9657586 | 12640288 | 0.5 | RS | rs10491744 | 12710106 | 0.66 | RS |
| rs10809811 | 12640996 | 0.587 | RS | rs10960760 | 12710152 | 0.66 | RS |
| rs1408794 | 12641340 | 0.587 | RS | rs2382361 | 12710786 | 0.66 | RS |
| rs1408795 | 12641413 | 0.807 | RS | rs1409626 | 12710820 | 0.66 | RS |
| rs13294940 | 12642364 | 0.586 | RS | rs1409630 | 12711251 | 0.679 | RS |
| rs995263 | 12644578 | 0.542 | RS | rs13288475 | 12711714 | 0.679 | RS |
| rs7022317 | 12656686 | 0.727 | RS | rs13288636 | 12711806 | 0.679 | RS |
| rs1121541 | 12657049 | 0.588 | RS | rs13288681 | 12711881 | 0.679 | RS |
| rs10960748 | 12658805 | 0.637 | RS | rs1326798 | 12712227 | 0.679 | RS |
| rs10960749 | 12661566 | 0.636 | RS | rs7871257 | 12712357 | 0.649 | RS |
| rs13294134 | 12663636 | 0.636 | RS | rs12379260 | 12713112 | 0.679 | RS |
| rs16929340 | 12664124 | 0.546 | RS | rs13284453 | 12714280 | 0.618 | RS |
| rs13299830 | 12664531 | 0.629 | RS | rs13284898 | 12714560 | 0.677 | RS |
| rs10960751 | 12665264 | 0.588 | RS | rs10960774 | 12729313 | 0.549 | RS |
| rs10960752 | 12665284 | 0.588 | RS | rs10738290 | 12730906 | 0.507 | RS |
| rs10960753 | 12665522 | 0.589 | RS | rs10756406 | 12738587 | 0.522 | RS |
| rs13296454 | 12667181 | 0.586 | RS | rs927869 | 12738962 | 0.522 | RS |
| rs13297008 | 12667471 | 0.586 | RS | rs4741245 | 12739300 | 0.522 | RS |
| rs10116013 | 12667979 | 0.631 | RS | rs7023927 | 12739596 | 0.522 | RS |
| rs10809826 | 12672663 | 0.668 | RS | rs7035500 | 12740095 | 0.522 | RS |
| rs13293905 | 12675943 | 0.856 | RS | rs13302551 | 12740812 | 0.543 | RS |
| rs2762460 | 12686478 | 0.679 | RS | rs1543587 | 12741741 | 0.522 | RS |
| rs2762461 | 12686499 | 0.733 | RS | rs1074789 | 12742340 | 0.51 | RS |
| rs2762462 | 12689776 | 0.687 | RS | rs10960779 | 12748881 | 0.508 | RS |
| rs2762463 | 12691897 | 0.914 | RS | | | | |

rs1800407 OCA2

| | | | | | | | |
|---|---|---|---|---|---|---|---|
| rs9920172 | 25874249 | 0.537 | RS | rs12910433 | 25902239 | 0.527 | RS |
| rs11638265 | 25876168 | 0.562 | RS | rs1900758 | 25903692 | 0.534 | RS |
| rs1800411 | 25885516 | 0.516 | RS | rs11630828 | 25911161 | 0.824 | RS |
| rs1448488 | 25890452 | 0.516 | RS | rs7178315 | 25911504 | 0.817 | RS |
| rs11636005 | 25894342 | 0.516 | RS | rs735067 | 25912497 | 0.817 | RS |
| rs11634923 | 25894631 | 0.516 | RS | rs2015343 | 25912896 | 0.817 | RS |
| rs7182323 | 25894924 | 0.516 | RS | rs8029026 | 25913305 | 0.817 | RS |
| rs11631735 | 25896375 | 0.516 | RS | rs2077596 | 25913330 | 0.817 | RS |
| rs12914687 | 25900136 | 0.516 | RS | rs8024822 | 25913899 | 0.816 | RS |
| rs12903382 | 25900544 | 0.516 | RS | rs11636259 | 25920585 | 0.817 | RS |

rs4778232 OCA2

| | | | | | | | |
|---|---|---|---|---|---|---|---|
| rs749846 | 25942585 | 0.59 | RS | rs7163354 | 25967383 | 0.963 | RS |
| rs3794606 | 25942603 | 0.999 | RS | rs1597196 | 25968517 | 0.779 | RS |
| rs1448485 | 25956336 | 0.527 | RS | rs6497254 | 25970020 | 0.963 | RS |
| rs7177686 | 25960939 | 0.963 | RS | rs895829 | 25971652 | 0.952 | RS |
| rs1470608 | 25961716 | 0.566 | RS | rs6497256 | 25973011 | 0.952 | RS |
| rs6497253 | 25962144 | 0.963 | RS | rs1562587 | 25976547 | 0.504 | RS |
| rs7170869 | 25962343 | 0.566 | RS | rs7179994 | 25997365 | 0.547 | RS |
| rs1375164 | 25965407 | 0.963 | RS | rs4778137 | 26001430 | 0.546 | RS |
| rs12442147 | 25965773 | 0.525 | RS | | | | |

rs8024968 OCA2

| | | | | | | | |
|---|---|---|---|---|---|---|---|
| rs749846 | 25942585 | 0.678 | RS | rs16950821 | 25957102 | 1 | RS |
| rs12441727 | 25945370 | 0.937 | RS | rs12324648 | 25960388 | 1 | RS |
| rs3794604 | 25945660 | 0.937 | RS | rs1470608 | 25961716 | 0.723 | RS |
| rs3794603 | 25945919 | 0.937 | RS | rs7170869 | 25962343 | 0.723 | RS |
| rs4778231 | 25949626 | 0.939 | RS | rs12442147 | 25965773 | 0.782 | RS |
| rs972335 | 25950596 | 0.939 | RS | rs1597196 | 25968517 | 0.528 | RS |
| rs17680684 | 25955691 | 0.939 | RS | rs1562587 | 25976547 | 0.678 | RS |
| rs1448485 | 25956336 | 0.764 | RS | | | | |

rs7495174 OCA2

| | | | | | | | |
|---|---|---|---|---|---|---|---|
| rs7174027 | 26002360 | 0.694 | RS | rs2240204 | 26167627 | 0.625 | RS |
| rs12593163 | 26003963 | 0.72 | RS | rs2240203 | 26167797 | 0.617 | RS |
| rs4778236 | 26006128 | 0.695 | RS | rs6497292 | 26169790 | 0.617 | RS |
| rs12593929 | 26032853 | 0.777 | RS | rs16950941 | 26176339 | 0.625 | RS |
| rs8025035 | 26051367 | 0.77 | RS | rs2240202 | 26184490 | 0.625 | RS |
| rs7497759 | 26089800 | 0.629 | RS | rs2016277 | 26191564 | 0.614 | RS |
| rs8041209 | 26117253 | 0.62 | RS | rs2016236 | 26192164 | 0.614 | RS |
| rs8182028 | 26141530 | 0.625 | RS | rs16950979 | 26194101 | 0.625 | RS |
| rs8182077 | 26141565 | 0.625 | RS | rs2346051 | 26196197 | 0.625 | RS |
| rs12592363 | 26160924 | 0.625 | RS | rs2346050 | 26196279 | 0.614 | RS |
| rs8028689 | 26162483 | 0.617 | RS | rs16950987 | 26199823 | 0.614 | RS |
| rs16950927 | 26163963 | 0.625 | RS | rs12592730 | 26203954 | 0.625 | RS |

rs7183877 HERC2

| | | | | | | | |
|---|---|---|---|---|---|---|---|
| rs12591531 | 26101511 | 1 | RS | rs16950949 | 26180428 | 0.97 | RS |
| rs6497287 | 26113882 | 0.998 | RS | | | | |

rs1667394 HERC2

| | | | | | | | |
|---|---|---|---|---|---|---|---|
| rs12913832 | 26039213 | 0.653 | RS | rs3940272 | 26142318 | 0.846 | RS |
| rs3935591 | 26047607 | 0.765 | RS | rs11631797 | 26175874 | 0.849 | RS |
| rs7170852 | 26101581 | 0.832 | RS | rs916977 | 26186959 | 1 | RS |
| rs2238289 | 26126810 | 0.803 | RS | rs8039195 | 26189679 | 0.849 | RS |

rs12592730 HERC2

| | | | | | | | |
|---|---|---|---|---|---|---|---|
| rs7495174 | 26017833 | 0.625 | RS | rs2240203 | 26167797 | 0.988 | RS |
| rs12593929 | 26032853 | 0.779 | RS | rs6497292 | 26169790 | 0.987 | RS |
| rs8025035 | 26051367 | 0.778 | RS | rs16950941 | 26176339 | 1 | RS |
| rs7497759 | 26089800 | 0.973 | RS | rs2240202 | 26184490 | 1 | RS |
| rs8041209 | 26117253 | 0.985 | RS | rs2016277 | 26191564 | 0.984 | RS |
| rs8182028 | 26141530 | 1 | RS | rs2016236 | 26192164 | 0.984 | RS |
| rs8182077 | 26141565 | 1 | RS | rs16950979 | 26194101 | 1 | RS |
| rs12592363 | 26160924 | 1 | RS | rs2346051 | 26196197 | 1 | RS |
| rs8028689 | 26162483 | 0.988 | RS | rs2346050 | 26196279 | 0.984 | RS |
| rs16950927 | 26163963 | 1 | RS | rs16950987 | 26199823 | 0.984 | RS |
| rs2240204 | 26167627 | 1 | RS | | | | |

RS means Rotterdam cohort (Hofman A et al (1991) Eur J Epidemiol 7: 403-422). HapMap CEU means Utah residents with Northern and Western European ancestry from the HapMap database (The International HapMap Project. Nature (2003) 426: 789-796; http://www.hapmap.org). HapMap CEU data are only included for SNPs that were not detected in the Rotterdam cohort.

Likewise, rs12896399 contributes less to the predictive accuracy of the method compared to rs12913832. The method will still provide an adequate predictive accuracy if a polymorphic site having a lower degree of linkage disequilibrium is to be used in place of rs12896399. A suitable polymorphic site which may be used in place of rs12896399 is one which is in linkage disequilibrium with rs12896399 at an r² value of at least 0.5, suitably at least 0.6, at least 0.7, at least 0.8, at least 0.9 or at least 0.95. Known SNPs having the required linkage disequilibrium with rs12896399 are listed in Table 1.

The method may involve genotyping polymorphisms which are yet to be identified. If a new polymorphism e.g. SNP is identified, it is straightforward to determine the LD with a known SNP by genotyping both polymorphisms in at least about 100 unrelated individuals in a population and using standard formulas. The r² value can be calculated using standard formulas when haplotypes between 2 SNPs are known. Haplotypes can be inferred from genotype data. For population data, the Expectation Maximization algorithm based programs such as haplo.stats (software website: http://mayoresearch.mayo.edu/mayo/research/schaid_lab/software.cfm; algorithm reference: Schaid D J, Rowland C M, Tines D E, Jacobson R M, Poland G A. (2002) Score tests for association between traits and haplotypes when linkage phase is ambiguous. Am J Hum Genet, 70: 425-434) can be used. For pedigree data, linkage based programs such as Merlin (software website: http://www.sph.umich.edu/csg/abecasis/MERLIN; algorithm reference: Abecasis et al. (2001) Merlin—rapid analysis of dense genetic maps using sparse gene flow trees. Nat Genet, 30: 97-101). New polymorphisms having high LD with a known SNP, such as an r² value of at least 0.5 or at least 0.9, may be found within 200 kb of the known SNP on the chromosome, such as within 100 kb, or 50 kb, or within the same linkage block. Locations of the SNPs useful in the invention, linkage blocks and broader chromosomal regions encompassing 100 kb upstream and downstream of each SNP are shown in Table 2.

TABLE 2
Chromosomal regions which may encompass polymorphisms in LD with SNPs
useful in the invention:

| SNP | Gene | Chr | Position | Linkage block | SNP location +/−100 kb |
|---|---|---|---|---|---|
| rs12913832 | HERC2 | 15 | 26039213 | 26032853-26051367 | 25939213-26139213 |
| rs1800407 | OCA2 | 15 | 25903913 | 25874249-25908005 | 25803913-26003913 |
| rs12896399 | SLC24A4 | 14 | 91843416 | 91830169-91875964 | 91743416-91943416 |
| rs16891982 | SLC45A2 | 5 | 33987450 | 33976176-34024292 | 33887450-34087450 |
| rs1393350 | TYR | 11 | 88650694 | 88622366-88677584 | 88550694-88750694 |
| rs12203592 | IRF4 | 6 | 341321 | 328546-348470 | 241321-441321 |
| rs12592730 | HERC2 | 15 | 26203954 | 26101511-26203954 | 26103954-26303954 |
| rs7495174 | OCA2 | 15 | 26017833 | 26001430-26029250 | 25917833-26117833 |
| rs1667394 | HERC2 | 15 | 26203777 | 26101511-26203954 | 26103777-26303777 |
| rs7183877 | HERC2 | 15 | 26039328 | 26032853-26051367 | 25939328-26139328 |
| rs4778232 | OCA2 | 15 | 25955360 | 25942603-25973069 | 25855360-26055360 |
| rs1408799 | TYRP1 | 9 | 12662097 | 12658121-12664124 | 12562097-12762097 |
| rs8024968 | OCA2 | 15 | 25957284 | 25942603-25973069 | 25857284-26057284 |
| rs683 | TYRP1 | 9 | 12699305 | 12672663-12706172 | 12599305-12799305 |

If the method of iris color prediction involves genotyping only the SNPs rs12913832, rs1800407 and rs12896399 (or polymorphic sites which are in linkage disequilibrium with one of those SNPs at the required r² value), it is preferable to identify the race of the human from whom the nucleic acid sample was obtained. The prediction accuracy is better for persons of European descent, e.g. for Caucasians. The European descent of an unknown person can be determined using ancestry-sensitive DNA markers as described in Lao et al AJHG 2008, Vol 78, 680-690; and Kersbergen et al. 2009 BMC Genetics 10:69. Ancestry can also be inferred from skull morphometry.

In one embodiment, the method further comprises genotyping the nucleic acid for at least one polymorphism selected from the group consisting of:
(i) the SNP rs16891982 or a polymorphic site which is in linkage disequilibrium with rs16891982 at an r² value of at least 0.5;
(ii) the SNP rs1393350 or a polymorphic site which is in linkage disequilibrium with rs1393350 at an r² value of at least 0.5;
(iii) the SNP rs12203592 or a polymorphic site which is in linkage disequilibrium with rs12203592 at an r² value of at least 0.5.

Suitably in this embodiment, each of the above polymorphisms is genotyped. rs16891982 is in the SLC45A2 gene; rs1393350 is in the TYR gene; and rs12203592 is in the IRF4 gene. Further information about these SNPs, including the major and minor alleles and their chromosomal locations, is provided in Example 1.

The embodiment of the method in which each of rs12913832 in HERC2; rs1800407 in OCA2; rs12896399 in SLC24A4; rs16891982 in SLC45A2; rs1393350 in TYR; and rs12203592 in IRF4 is used for prediction of iris color is exemplified in Example 2. The prediction accuracy is greater when these six SNPs are used in prediction than when only the top three are used i.e. rs12913832, rs1800407 and rs16891982. Also, the prediction is accurate irrespective of the ancestry of the human subject. This is the case also where rs12913832 in HERC2; rs1800407 in OCA2; rs12896399 in SLC24A4; rs16891982 in SLC45A2 are used. Hence, additional testing to determine the race or bio-geographic ancestry of a person is not necessary for correct interpretation of the prediction results, providing a clear advantage in practical forensic applications.

A suitable polymorphic site which may be used in place of rs16891982 is one which is in linkage disequilibrium with rs16891982 at an r² value of at least 0.5, suitably at least 0.6, at least 0.7, at least 0.8, at least 0.9 or at least 0.95. Known SNPs having the required linkage disequilibrium with rs16891982 are listed in Table 1.

A suitable polymorphic site which may be used in place of rs1393350 is one which is in linkage disequilibrium with rs1393350 at an r² value of at least 0.5, suitably at least 0.6, at least 0.7, at least 0.8, at least 0.9 or at least 0.95. Known SNPs having the required linkage disequilibrium with rs1393350 are listed in Table 1.

A suitable polymorphic site which may be used in place of rs12203592 is one which is in linkage disequilibrium with rs12203592 at an r² value of at least 0.5, suitably at least 0.6, at least 0.7, at least 0.8, at least 0.9 or at least 0.95.

A further increase in prediction accuracy can be achieved when further polymorphisms are genotyped. In this embodiment, the method further comprises genotyping the nucleic acid for at least one polymorphism selected from the group consisting of:
(i) the SNP rs12592730 or a polymorphic site which is in linkage disequilibrium with rs12592730 at an r² value of at least 0.5;
(ii) the SNP rs7495174 or a polymorphic site which is in linkage disequilibrium with rs7495174 at an r² value of at least 0.5;
(iii) the SNP rs1667394 or a polymorphic site which is in linkage disequilibrium with rs1667394 at an r² value of at least 0.5;
(iv) the SNP rs7183877 or a polymorphic site which is in linkage disequilibrium with rs7183877 at an r² value of at least 0.5;
(v) the SNP rs4778232 or a polymorphic site which is in linkage disequilibrium with rs4778232 at an r² value of at least 0.5;
(vi) the SNP rs1408799 or a polymorphic site which is in linkage disequilibrium with rs1408799 at an r² value of at least 0.5;
(vii) the SNP rs8024968 or a polymorphic site which is in linkage disequilibrium with rs8024968 at an r² value of at least 0.5;
(viii) the SNP rs683 or a polymorphic site which is in linkage disequilibrium with rs683 at an r² value of at least 0.5;

Further information about these SNPs, including the major and minor alleles and their chromosomal locations is provided in Example 1. For each of the above SNPs, where an alternative polymorphic site is used, it should be in LD with an r² value of at least 0.5, suitably at least 0.6, at least 0.7, at least 0.8, at least 0.9 or at least 0.95. Suitable SNPs are indicated in Table 1.

According to the method of the first aspect of the invention, the prediction of iris color involves genotyping appropriate polymorphisms as discussed above, and comparing the combination of the genotypes of the polymorphisms to known relationships of genotype and iris color. Thus, the iris color may be inferred from the genotypes of the polymorphisms that have been analyzed.

Methods for performing such a comparison and reaching a conclusion based on that comparison are exemplified herein. The inference typically involves using a complex model that involves using known relationships of known alleles or nucleotide occurrences as classifiers. Such a model is a “prediction model”. Various methods can be used to arrive at a prediction model. As illustrated in Example 1, ordinal regression, multinomial logistic regression, fuzzy c-means clustering, neural networks or classification trees may be used to generate a prediction model. The skilled person may develop alternative prediction models.

Of the prediction models tested, the multinomial logistic regression model described in Examples 1 and 2 was found to be most accurate. One way of implementing the method is therefore to genotype the necessary polymorphisms and apply the multinomial logistic regression model described in Examples 1 and 2 to make the prediction.

The alpha and beta model parameters for the multinomial logistic regression model as applied to various combinations of SNPs are shown in Table 3, together with AUC, an indication of prediction accuracy for each category.

TABLE 3
Alpha and beta model parameters and expected AUC

| | | | Expected | | |
|---|---|---|---|---|---|
| | | | AUC | | |
| | beta1 | beta2 | Blue | Inter | Brown |
| alpha | 3.9353 | 0.5535 | 0.9036 | 0.7062 | 0.916 |
| rs12913832 | −4.8074 | −1.8335 | | | |
| rs1800407 | 1.381 | 1.0454 | | | |
| rs12896399 | −0.5486 | −0.0185 | | | |
| alpha | 4.0103 | 0.5798 | 0.9064 | 0.71 | 0.9184 |
| rs12913832 | −4.8169 | −1.8161 | | | |
| rs1800407 | 1.3676 | 0.9991 | | | |
| rs12896399 | −0.5463 | −0.0061 | | | |
| rs16891982 | −1.2567 | −0.6575 | | | |
| alpha | 3.7547 | 0.4446 | 0.9063 | 0.71 | 0.9184 |
| rs12913832 | −4.8532 | −1.8563 | | | |
| rs1800407 | 1.4047 | 1.0577 | | | |
| rs12896399 | −0.5391 | −0.0096 | | | |
| rs1393350 | 0.4212 | 0.2587 | | | |
| alpha | 3.9057 | 0.529 | 0.9022 | 0.7112 | 0.9169 |
| rs12913832 | −4.93 | −1.901 | | | |
| rs1800407 | 1.4319 | 1.0553 | | | |
| rs12896399 | −0.5801 | −0.0435 | | | |
| rs12203592 | 0.6467 | 0.7032 | | | |
| alpha | 3.8339 | 0.4703 | 0.9096 | 0.7147 | 0.9214 |
| rs12913832 | −4.8608 | −1.8406 | | | |
| rs1800407 | 1.3893 | 1.012 | | | |
| rs12896399 | −0.5373 | 0.0022 | | | |
| rs16891982 | −1.2441 | −0.6421 | | | |
| rs1393350 | 0.4101 | 0.2606 | | | |
| alpha | 3.9643 | 0.7024 | 0.9121 | 0.7234 | 0.9288 |
| rs12913832 | −4.831 | −1.8101 | | | |
| rs1800407 | 1.4291 | 0.9083 | | | |
| rs12896399 | −0.58 | −0.0287 | | | |
| rs16891982 | −1.284 | −0.5203 | | | |
| rs1393350 | 0.4665 | 0.2608 | | | |
| rs12203592 | 0.6638 | 0.6964 | | | |
| rs12592730 | 1.4712 | 0.4671 | | | |
| rs7495174 | −0.985 | −0.3821 | | | |
| rs1667394 | −1.015 | −0.3168 | | | |
| rs7183877 | 0.9085 | 0.3543 | | | |
| rs4778232 | 0.4195 | 0.2237 | | | |
| rs1408799 | −0.242 | −0.0849 | | | |
| rs8024968 | −0.251 | −0.4482 | | | |
| rs683 | −0.134 | −0.2955 | | | |

The effect alleles to which the model parameters are applied are the minor alleles as indicated in Table 5.

According to one embodiment, a polymorphism which is in LD with one of the SNPs mentioned in relation to the first aspect of the invention, i.e. rs12913832, rs1800407, rs12896399, rs16891982, rs1393350, rs12203592, rs12592730, rs7495174, rs1667394, rs7183877, rs4778232, rs1408799, rs8024968 or rs683, may be genotyped in place of the corresponding SNP. To use the information from such a polymorphism in the prediction method, it may be necessary to build a modified prediction model based on genotype and phenotype data (either Rotterdam cohort data as described in the Examples or other available data or new data). The modified prediction model can be developed using the statistical techniques described in Example 1.

Typically, the method provides a categorical prediction of the iris color. Suitably, the categories are brown, blue and intermediate. Another possible categorisation could be between blue and non-blue or between brown and non-brown. The exemplified method provides for a categorical prediction of brown, blue or intermediate. “Brown” includes all hues and all shades or tints of brown. “Blue” includes all hues and all shades or tints of gray or blue. “Intermediate” includes hazel, or green iris color. When developing a model, assignment of an eye color category for the model building data set can be done on the basis of inspection of eye photographs. The use of good quality photographic images, several images per eye and categorisation by a single grader are preferred.

Typically, a categorical prediction may return a probability of a true positive for each of the categories, the probabilities adding up to 1. Suitably, the category which has the highest probability of a true positive would be the category in which the iris color is predicted. For example, the probability may be 0.90 for blue, 0.06 for intermediate and 0.04 for brown. In that case, the prediction would be that the iris color is blue. If the probability of blue was, say, only 0.70, the degree of confidence that the prediction is correct would be lower. In particular, there would be a greater probability of a false positive, i.e. blue is predicted but the color is actually not blue. One can set a minimum probability below which the prediction is unclassified. For example, if one set a minimum probability of 0.80, in the case in which blue is predicted at 0.90, the prediction would remain blue. In the second case, where the probability of blue was only 0.70, the prediction would be unclassified. Different degrees of sensitivity and specificity would be associated with each probability (accuracy) level. “Sensitivity” is the correct call rate and equals 100% minus the percentage of false negatives. “Specificity” equals 100% minus the percentage of false positives. Historical data may be used to establish the sensitivity and specificity of the prediction at a given probability level. Altering the probability level can achieve higher specificity levels although this well affect the overall sensitivity of the model. Thus, as well as returning the category, whether it be blue, intermediate, brown or unclassified, the method can also involve recording the probability of a true positive in that category, and/or the probability level used as the cut-off, and/or the specificity and/or sensitivity of the model for the given probability level.

By ‘genotyping’, we include determining the genotype of at least one of the SNPs described herein. In this way, the particular base or allele of a polymorphic site (e.g. SNP) becomes known. It is appreciated that by ‘genotyping’ we include the direct determination of a particular base or allele of a polymorphic site, as well as an indirect indicator of a particular base or allele of a polymorphic site.

It will be appreciated that genotyping a polymorphic site (e.g. SNP) as described above conveniently comprises contacting a sample of nucleic acid from the human with one or more nucleic acid molecules that hybridize selectively to a genomic region encompassing the polymorphism (e.g. SNP).

By “selective hybridization” or “selectively hybridize” we include the meaning that the nucleic acid molecule has sufficient nucleotide sequence similarity with the said genomic DNA or cDNA or mRNA that it can hybridize under highly stringent conditions. As is well known in the art, the stringency of nucleic acid hybridisation depends on factors such as length of nucleic acid over which hybridisation occurs, degree of identity of the hybridizing sequences and on factors such as temperature, ionic strength and CG or AT content of the sequence. Conditions that allow for selective hybridization can be determined empirically, or can be estimated based, for example, on the above parameters (see, for example, Sambrook et al., “Molecular Cloning: A laboratory manual (Cold Spring Harbor Laboratory Press 1989)). Thus, any nucleic acid which is capable of selectively hybridizing as said is useful in the practice of the invention.

An example of a typical hybridization solution when a nucleic acid is immobilised on a nylon membrane and the probe is an oligonucleotide of between 15 and 50 bases is:

3.0 M trimethylammonium chloride (TMACl)
0.01 M sodium phosphate (pH 6.8)

### 1 mm EDTA (pH 7.6)

### 0.5% SDS

100 μg/ml denatured, fragmented salmon sperm DNA
0.1% nonfat dried milk

The optimal temperature for hybridisation is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50° C.; for 19-mers, it is 55-57° C.; and for 20-mers, it is 58-66° C.

Nucleic acids which can selectively hybridize to the said DNA (such as human DNA) include nucleic acids which have >95% sequence identity, preferably those with >98%, more preferably those with >99% sequence identity, for example 100% sequence identity, over at least a portion of the nucleic acid with the said DNA or cDNA. As is well known, human genes usually contain introns such that, for example, a mRNA or cDNA derived from a gene within the said human DNA would not match perfectly along its entire length with the said human DNA but would nevertheless be a nucleic acid capable of selectively hybridizing to the said human DNA. Thus, the invention specifically includes nucleic acids which selectively hybridize to a cDNA but may not hybridize to the corresponding gene, or vice versa. For example, nucleic acids which span the intron-exon boundaries of a given gene may not be able to selectively hybridize to the cDNA of the gene. The nucleic acid may selectively hybridize to the said DNA over substantially the entire length of the nucleic acid, or only a portion of it may selectively hybridize, i.e. the hybridizing portion.

Typically, the one or more nucleic acid molecules that hybridize selectively to a genomic region encompassing the polymorphism are less than 100 bases in length, such as less than 90, 80, 70, 60, 50, 40 or 30 bases. Typically, the hybridizing portion is less than 100 bases in length, such as less than 90, 80, 70, 60, 50, 40 or 30 bases. Typically, the hybridizing portion may be between 10 and 30 bases in length, such as 11, 12, 13, 14, 15, 16, 17, 18, 19, 20, 21, 22, 23, 24, 25, 26, 27, 28 or 29 bases in length. The nucleic acid molecule may comprise one or more regions which do not hybridize selectively to said genomic region. Such regions may be useful for distinguishing between different nucleic acid molecules in a population of nucleic acid molecules. For example, the nucleic acid molecules used to genotype SNPs in Example 2 comprise 5′ non-hybridizing portions of different numbers of “t” residues. The nucleic acid molecules are distinguished by virtue of their differing molecular weights, which in turn depends on the number of “t” residues.

“Nucleic acid that hybridizes selectively” is typically nucleic acid which will amplify DNA from the said region of DNA by any of the well known amplification systems such as those described in more detail below, in particular the polymerase chain reaction (PCR). Suitable conditions for PCR amplification include amplification in a suitable 1× amplification buffer:

10× amplification buffer is 500 mM KCl; 100 mM Tris.Cl (pH 8.3 at room temperature); 15 mM MgCl₂; 0.1% gelatin.

A suitable denaturing agent or procedure (such as heating to 95° C.) is used in order to separate the strands of double-stranded DNA.

Suitably, the annealing part of the amplification is between 37° C. and 60° C., preferably 50° C.

By ‘hybridizing selectively to a genomic region encompassing the polymorphism’ we include hybridizing at or near the polymorphism. The nucleic acid molecule may hybridize equally to the genomic region irrespective of the identity of the allele, or it may hybridize differentially to a genomic region encompassing one allele of a polymorphic site (e.g. SNP) versus another allele of that polymorphic site (e.g. SNP).

The “genomic region encompassing a polymorphism” can be considered as the polymorphism itself and its upstream and/or downstream flanking nucleotide sequences. The latter can serve to aid in the identification of the precise location of the SNP in the human genome, and serve as target gene segments useful for performing methods of the invention. Primers and probes that selectively hybridize to either or both flanking nucleotide sequences and optionally also the polymorphism, can be designed based on the disclosed gene sequences and information provided herein.

Typically, the sample of nucleic acid which is analysed is one which has been amplified from the immediate sample obtained from the human. Any of the nucleic acid amplification protocols can be used including the polymerase chain reaction, QB replicase and ligase chain reaction. Also, NASBA (nucleic acid sequence based amplification), also called 3SR, can be used as described in Compton (1991) Nature 350, 91-92 and AIDS (1993), Vol 7 (Suppl 2), S108 or SDA (strand displacement amplification) can be used as described in Walker et al (1992) Nucl. Acids Res. 20, 1691-1696. The polymerase chain reaction is particularly preferred because of its simplicity. Thus it will be appreciated that the sample of the nucleic acid of the human may be subjected to a nucleic acid amplification before genotyping or as part of the genotyping method. Typically, the amplification will be directed to the polymorphisms of interest using appropriate primer pairs.

Numerous methods are known in the art for genotyping a polymorphism, and particularly for determining the nucleotide occurrence for a particular SNP in a sample. Such methods can utilize one or more oligonucleotide probes or primers, including, for example, an amplification primer pair that selectively hybridize to a genomic region encompassing a polymorphism (e.g. SNP). Oligonucleotide probes useful in practicing a method of the invention can include, for example, an oligonucleotide that is complementary to and spans a portion of the genomic region encompassing the SNP, including the position of the SNP, wherein the presence of a specific nucleotide at the position (i.e., the SNP) is detected by differential hybridization of the probe, such as by the presence or absence of selective hybridization of the probe. Such a method can further include contacting the genomic region encompassing the polymorphism and hybridized oligonucleotide with an endonuclease, and detecting the presence or absence of a cleavage product of the probe, depending on whether the nucleotide occurrence at the SNP site is complementary to the corresponding nucleotide of the probe. Ye et al 2002 J Forensic Sci 47:592-600 describe how differential hybridization of a probe depending on the allele of a polymorphism can be determined by melting curve analysis.

An oligonucleotide ligation assay also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP locus can be sequenced using traditional sequence methodologies (e.g., the “dideoxy-mediated chain termination method,” also known as the “Sanger Method” (Sanger, F., et al., J. Molec. Biol. 94:441 (1975); Prober et al. Science 238:336-340 (1987)) and the “chemical degradation method,” “also known as the “Maxam-Gilbert method” (Maxam, A. M., et al., Proc. Natl. Acad. Sci. (U.S.A.) 74:560 (1977)), both references herein incorporated by reference) to determine the nucleotide occurrence at the SNP loci.

Methods of the invention can identify nucleotide occurrences at SNPs using a “microsequencing” method. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP locus are discussed in Boyce-Jacino et al., U.S. Pat. No. 6,294,336, incorporated herein by reference, and summarized herein.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher et al, Nucl. Acids. Res. 17:7779-7784 (1989); Sokolov, Nucl. Acids Res. 18:3671 (1990); Syvanen, et al., Genomics 8:684-692 (1990); Kuppuswamy et al., Proc. Natl. Acad. Sci. (U.S.A.) 88:1143-1147 (1991); Prezant et al, Hum. Mutat. 1:159-164 (1992); Ugozzoli et al., GATA 9:107-112 (1992); Nyren et al., Anal. Biochem. 208:171-175 (1993); and Wallace, WO 89/10414). These methods differ from Genetic Bit™ method of analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. Amer. J. Hum. Genet. 52:46-59 (1993)). Alternative microsequencing methods have been provided by Mundy (U.S. Pat. No. 4,656,127) and Cohen, D. et al (French Patent 2,650,840; PCT Appl. No. WO91/02087) which discusses a solution-based method for determining the identity of the nucleotide of a polymorphic site. As in the Mundy method of U.S. Pat. No. 4,656,127, a primer is employed that is complementary to allelic sequences immediately 3′- to a polymorphic site.

Boyce-Jacino et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

In one particular commercial example of a method that can be used to identify a nucleotide occurrence of one or more SNPs, the nucleotide occurrences of SNPs in a sample can be determined using the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide triphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Orchid BioSciences, Inc., Princeton, N.J.).

It will be appreciated that the methods of the invention may also be carried out on “DNA chips”. Such “chips” are described in U.S. Pat. No. 5,445,934 (Affymetrix; probe arrays), WO 96/31622 (Oxford Gene Technology; probe array plus ligase or polymerase extension), and WO 95/22058 (Affymax; fluorescently marked targets bind to oligomer substrate, and location in array detected); all of these are incorporated herein by reference.

PCR amplification of small regions (for example up to 300 bp) can be used to detect small changes greater than 3-4 bp insertions or deletions. Amplified sequence may be analysed on a sequencing gel, and small changes (minimum size 3-4 bp) can be visualised. Suitable primers are designed as herein described.

In one embodiment, the method of genotyping a polymorphism comprises performing a primer extension reaction and detecting the primer extension reaction product. Suitably, the primer extension reaction is a multiplex primer extension reaction. In such a reaction, the primers themselves or the extension products of the different primers are distinguishable from each other. For example, they may be distinguishable by virtue of molecular size (for example as in the ABI Prism® SNaPshot™ Multiplex assay as described below), the presence of a unique tag in each primer which allows binding to appropriately located complementary nucleic acid molecules on a solid substrate (see Hirshchorn et al 2000 Proc Natl Acad Sci USA 97: 12164-12169), or by virtue of their individualised location on a solid substrate (see Krjut\check{s}kov et al 2008 Nucleic Acids Res 36: e75.

A suitable method is the ABI Prism® SNaPshot™ Multiplex assay (Applied Biosystems, CA, USA) as used in the Examples. Multiplex PCR is used to amplify the genomic regions encompassing several SNPs in a single PCR. For each PCR product, a primer which hybridizes selectively to the PCR product is used in a single base extension (SBE) reaction. Each primer has a 5′ non-hybridizing region containing an appropriate number of T residues such that each SBE reaction product has a different molecular size to allow unequivocal detection when several SNPs are included in a single (multiplex) SBE reaction. The single base extension (SBE) reaction is performed to introduce a dye-labelled ddNTP complementary to the allele of each target SNP and the products are then separated by electrophoresis and the dye detected using appropriate sensors. Alternative 5′ non-hybridizing regions may comprise A residues. Other suitable methods involving a primer extension are as discussed above.

A second aspect of the invention provides a method of preparing a data carrier containing data on the predicted iris color of a human, the method comprising recording the results of a method carried out according to the first aspect of the invention on a data carrier.

The data produced from carrying out the methods of the invention may conveniently be recorded on a data carrier. Thus, the invention includes a method of recording data on the predicted iris color of a human using any of the methods of the invention and recording the results on a data carrier. Typically, the data are recorded in an electronic form and the data carrier may be a computer, a disk drive, a memory stick, a CD or DVD or floppy disk or the like.

Information recorded on the data carrier may include the genotype information obtained using the methods of the invention and/or the prediction of iris color. For example, if a categorical prediction is given, this may include the category of iris color, such as whether it be blue, intermediate, brown or unclassified, the probability of a true positive in that category, the probability level used as the cut-off, and/or the specificity and/or sensitivity of the model for the given probability level. Other identifying information may also be included, such as the date and location from which the nucleic acid sample was obtained.

A third aspect of the invention provides a method for predicting the iris color of a human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

The allele occurrences may typically be determined or have been determined by performing steps (a) and (b) of the method of the first aspect of the invention. The prediction of the iris color may then be made using step (c) of the first aspect of the invention.

A fourth aspect of the invention provides a method for creating a description of a human based on forensic testing, wherein the description includes a prediction of the iris color of the human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

The determination of the allele occurrences and the prediction of iris color may be made as described in relation to the third aspect of the invention. The description may include features in addition to the predicted iris color, such as the age or gender of the human, including features determined using further forensic tests. The age of unidentified corpses and skeletons, and also of living persons, can be evaluated using methods known in the art, as described in Schmeling et al, 2007, Forensic Sci Int. 165:178-81. Age may also be inferred from biological markers such as gene expression markers as described in Lu T et al (2004) Nature 429 (6994): 883-91, or from DNA methylation markers. Gender can be determined using genetic tests based on the presence or absence of markers indicative of the Y chromosome (Esteve Codina A et al (2009) Int J Legal Med 123: 459-464). Such a description of a human, particularly of a wanted person, may be useful in tracing the wanted person. A description of a person to be identified from their remains may be useful in identifying a potential relative of the person. Once a potential relative is identified, the genetic profile of the potential relative and the person's remains can be compared, to determine whether the two are in fact related.

A fifth aspect of the invention provides a method for genotyping polymorphisms indicative of human iris color comprising:
(a) obtaining a sample of the nucleic acid of a human; and
(b) genotyping the nucleic acid for at least the following polymorphisms:
   (i) the single nucleotide polymorphism (SNP) rs12913832 or a polymorphic site which is in linkage disequilibrium with rs12913832 at an r² value of at least 0.9;
   (ii) the SNP rs1800407 or a polymorphic site which is in linkage disequilibrium with rs1800407 at an r² value of at least 0.5; and,
   (iii) the SNP rs12896399 or a polymorphic site which is in linkage disequilibrium with rs12896399 at an r² value of at least 0.5.

The genotyping methods are as discussed in relation to the first aspect of the invention. Additional polymorphisms to those listed above, including some or all of those discussed in relation to the first aspect of the invention may also be genotyped according to this aspect of the invention.

A sixth aspect of the invention provides a kit of parts for use in predicting the iris color of a human comprising:
(i) a primer pair suitable for amplifying the genomic region encompassing the single nucleotide polymorphism (SNP) rs12913832 or a polymorphic site which is in linkage disequilibrium with rs12913832 at an r² value of at least 0.9;
(ii) a primer pair suitable for amplifying the genomic region encompassing the SNP rs1800407 or a polymorphic site which is in linkage disequilibrium with rs1800407 at an r² value of at least 0.5; and,
(iii) a primer pair suitable for amplifying the genomic region encompassing the SNP rs12896399 or a polymorphic site which is in linkage disequilibrium with rs12896399 at an r² value of at least 0.5.

Suitable primer pairs and amplification methods are as discussed in relation to the first aspect of the invention. Suitably, each of the primer pairs is suitable for use together in a multiplex polymerase chain reaction. The kit may be used in conjunction with the genotyping methods discussed in relation to the first aspect of the invention. Suitable primer pairs for amplifying genomic regions encompassing additional polymorphisms to those listed above, including some or all of those discussed in relation to the first aspect of the invention may also be included in the kit. The amplified regions may then be genotyped according to the first aspect of the invention.

A seventh aspect of the invention provides a kit of parts for use in predicting the iris color of a human comprising:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

Suitable nucleic acid molecules and methods of using them to genotype polymorphisms are as discussed in relation to the first aspect of the invention. Suitably, each of the nucleic acid molecules is a primer suitable for performing a primer extension reaction, suitably in a multiplex reaction. The kit may be used in conjunction with the kit of the sixth aspect of the invention. Suitable nucleic acid molecules that hybridize selectively to additional genomic region encompassing a polymorphism, including some or all of those discussed in relation to the first aspect of the invention, may also be included in the kit.

An eighth aspect of the invention provides a solid substrate for use in predicting the iris color of a human comprising, the solid substrate having attached thereto:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

The solid substrate with the nucleic acids attached thereto may be a DNA chip or a microarray. Typically, each array position on the DNA chip or microarray is attached to a nucleic acid molecule having a different sequence. Suitable chips and microarrays are as described above in relation to the first aspect of the invention. Suitably, each of the nucleic acid molecules is a primer suitable for performing a primer extension reaction.

In one embodiment, the solid substrate has only the nucleic acid molecules that hybridize as said attached thereto.

The solid substrate may be used in conjunction with the kit of the sixth aspect of the invention. Suitable nucleic acid molecules that hybridize selectively to additional genomic region encompassing a polymorphism, including some or all of those discussed in relation to the first aspect of the invention, may also be attached to the solid substrate.

The present invention will be further illustrated in the following examples, without any limitation thereto.

### Example 1

### Eye Color and the Prediction of Complex Phenotypes from Genotypes

Predicting complex human phenotypes from genotypes has recently gained tremendous interest in the emerging field of consumer genomics, particularly in light of attempting personalized medicine [1, 2]. So far however, this approach was never shown to be accurate, even in combination with non-DNA-based information, thereby limiting practical applications [3, 4]. Here, we used human eye (iris) color of Europeans as an empirical example to demonstrate that accurate genetic prediction of complex human phenotypes is feasible. Moreover, the six DNA markers we identified as major eye color predictors will be valuable in forensic studies.

Facilitated by recent genome-wide genotyping, single nucleotide polymorphisms (SNPs) in various genes have been identified to be unambiguously associated with human eye color variation in Europeans [5-7], affirming eye color as a genetically complex phenotype. Thus, eye color may be used to exemplify the feasibility of accurate genetic prediction of complex human phenotypes. Recent attempts in predicting eye color have obtained promising results using SNPs in OCA2 [15], or in combination with HERC2 [5], or additionally in SLC24A4 and TYR [6]. However, a number of genetic variants with strong eye color association were not used in the previous prediction analyses; most of them were only identified in parallel or later studies [7-10]. To investigate the power of DNA-based eye color prediction, we genotyped 37 SNPs from eight genes [5-15], representing all currently known genetic variants with statistically significant eye color association (Table 8 and Table 4), in a large population sample of 6168 Dutch Europeans from the Rotterdam Study [16] (67.6% blue eyes, 22.8% brown and 9.6% neither blue nor brown and categorized as intermediate color) and performed prediction analyses with several models and parameters. Population characteristics, phenotype collection, SNP ascertainment, genotyping methods and details of prediction models and parameters are described in the supplemental data.

TABLE 4
37 SNPs with significant iris color association as ascertained from previous
studies with details from the previous studies and the present one

| | | | | | Previous studies | | Rotterdam Study | | | |
|---|---|---|---|---|---|---|---|---|---|---|
| SNP-ID | Chr | Position | Gene | Allele | Reference¹⁾ | P-value | N | CR | MA | MAF |
| rs16891982 | 5 | 33987450 | SLC45A2(MATP) | CG | [S6] | 5.0E−03 | 6420 | 0.99 | C | 0.03 |
| rs26722 | 5 | 33999627 | SLC45A2(MATP) | CT | [S6] | 2.0E−03 | 6428 | 0.99 | T | 0.01 |
| rs12203592 | 6 | 341321 | IRF4 | CT | [S6] | 6.1E−13 | 5971²⁾ | 1.00 | T | 0.08 |
| rs1408799 | 9 | 12662097 | TYRP1 | CT | [S5] | 1.5E−09 | 5964²⁾ | 1.00 | T | 0.17 |
| rs683 | 9 | 12699305 | TYRP1 | AC | [S13] | <0.01 | 6367 | 0.98 | C | 0.32 |
| rs1393350 | 11 | 88650694 | TYR | AG | [S9] | 3.3E−12 | 6410 | 0.99 | A | 0.23 |
| rs12896399 | 14 | 91843416 | SLC24A4 | GT | [S5, S6, S9] | 4.1E−38 | 6409 | 0.99 | G | 0.50 |
| rs2594935 | 15 | 25858633 | OCA2 | AG | [S3] | 1.5E−10 | 6417 | 0.99 | A | 0.25 |
| rs728405 | 15 | 25873448 | OCA2 | AC | [S3] | 3.8E−09 | 6308 | 0.98 | C | 0.18 |
| rs1800407 | 15 | 25903913 | OCA2 | CT | [S7] | 5.0E−10 | 6219 | 0.97 | T | 0.04 |
| rs3794604 | 15 | 25945660 | OCA2 | CT | [S3] | 8.5E−12 | 6418 | 0.99 | T | 0.11 |
| rs4778232 | 15 | 25955360 | OCA2 | CT | [S3] | 2.5E−13 | 6411 | 0.99 | T | 0.22 |
| rs1448485 | 15 | 25956336 | OCA2 | GT | [S3] | 3.4E−08 | 6392 | 0.99 | T | 0.13 |
| rs8024968 | 15 | 25957284 | OCA2 | CT | [S3] | 1.5E−11 | 6430 | 0.99 | T | 0.10 |
| rs1597196 | 15 | 25968517 | OCA2 | GT | [S3] | 9.1E−18 | 6387 | 0.99 | T | 0.18 |
| rs7179994 | 15 | 25997365 | OCA2 | AG | [S3] | 5.4E−13 | 6417 | 0.99 | G | 0.14 |
| rs4778138 | 15 | 26009415 | OCA2 | AG | [S3, S11] | 5.4E−221 | 6421 | 0.99 | G | 0.12 |
| rs4778241 | 15 | 26012308 | OCA2 | AC | [S3, S8, S11] | 2.8E−267 | 6426 | 0.99 | A | 0.15 |
| rs7495174 | 15 | 26017833 | OCA2 | AG | [S3, S5, S9, | 1.4E−239 | 6407 | 0.99 | G | 0.06 |
| | | | | | S11] | | | | | |
| rs1129038 | 15 | 26030454 | HERC2 | CT | [S7, S8] | 6.1E−46 | 6412 | 0.99 | C | 0.18 |
| rs12593929 | 15 | 26032853 | HERC2 | AG | [S8] | —³⁾ | 6427 | 0.99 | G | 0.06 |
| rs12913832 | 15 | 26039213 | HERC2 | AG | [S6-S8] | 6.1E−46 | 6420 | 0.99 | A | 0.18 |
| rs7183877 | 15 | 26039328 | HERC2 | AC | [S3] | 6.2E−11 | 6407 | 0.99 | A | 0.05 |
| rs3935591 | 15 | 26047607 | HERC2 | CT | [S8] | 1.5E−25 | 6413 | 0.99 | T | 0.11 |
| rs7170852 | 15 | 26101581 | HERC2 | AT | [S8] | 1.1E−17 | 6421 | 0.99 | T | 0.13 |
| rs8041209 | 15 | 26117253 | HERC2 | GT | [S3] | 6.6E−22 | 6415 | 0.99 | T | 0.05 |
| rs8028689 | 15 | 26162483 | HERC2 | CT | [S3] | 1.2E−21 | 6426 | 0.99 | C | 0.05 |
| rs2240203 | 15 | 26167797 | HERC2 | CT | [S8] | 8.9E−17 | 6424 | 0.99 | C | 0.05 |
| rs2240202 | 15 | 26184490 | HERC2 | AG | [S3] | 2.2E−22 | 6412 | 0.99 | A | 0.05 |
| rs916977 | 15 | 26186959 | HERC2 | CT | [S3, S7, S8] | <1E−300 | 6420 | 0.99 | T | 0.12 |
| rs16950979 | 15 | 26194101 | HERC2 | AG | [S3] | 7.0E−11 | 6394 | 0.99 | G | 0.05 |
| rs2346050 | 15 | 26196279 | HERC2 | CT | [S3] | 6.3E−19 | 6413 | 0.99 | C | 0.05 |
| rs16950987 | 15 | 26199823 | HERC2 | AG | [S3] | 8.3E−11 | 6414 | 0.99 | A | 0.05 |
| rs1667394 | 15 | 26203777 | HERC2 | CT | [S3, S5, S7, | 8.5E−31 | 6405 | 0.99 | C | 0.13 |
| | | | | | S9] | | | | | |
| rs12592730 | 15 | 26203954 | HERC2 | AG | [S3] | 2.6E−22 | 6409 | 0.99 | A | 0.05 |
| rs1635168 | 15 | 26208861 | HERC2 | AC | [S3] | 1.5E−11 | 6397 | 0.99 | A | 0.06 |
| rs6058017 | 20 | 32320659 | ASIP | AG | [S10, S13] | 2.2E−03 | 6186 | 0.97 | G | 0.12 |

P-value for eye color association obtained from the largest previous study in case included in several studies; CR: call rate in the current study; MA: minor allele; MAF: minor allele frequency;
¹⁾see Supplemental Reference list,
²⁾data from Infinium II HumanHap550K Genotyping arrays,
³⁾in haplotype association with eye color

TABLE 5
Single-SNP association with human iris color variation from the Rotterdam Study
with and without adjustment for the largest effect contributed by HERC2 rs12913832,
Tagging SNP selection and priority rank in prediction analysis

| SNP | Gene | Chr | Position | minor | beta1 | P1 | beta2 | P2 | Tag | Rank |
|---|---|---|---|---|---|---|---|---|---|---|
| rs16891982 | SLC45A2 | 5 | 33987450 | C | 0.45 | 1.1E−30 | 0.08 | 3.7E−03 | 1 | 4 |
| rs26722 | SLC45A2 | 5 | 33999627 | T | 0.32 | 4.6E−06 | 0.13 | 4.1E−03 | 1 | |
| rs12203592 | IRF4 | 6 | 341321 | T | −0.07 | 7.5E−03 | −0.07 | 2.9E−05 | 1 | 6 |
| rs1408799 | TYRP1 | 9 | 12662097 | T | 0.05 | 3.3E−03 | 0.05 | 5.3E−05 | 1 | 12 |
| rs683 | TYRP1 | 9 | 12699305 | C | 0.07 | 5.6E−06 | 0.03 | 3.3E−03 | 1 | 15 |
| rs1393350 | TYR | 11 | 88650694 | A | −0.05 | 8.8E−03 | −0.05 | 3.8E−06 | 1 | 5 |
| rs12896399 | SLC24A4 | 14 | 91843416 | G | 0.09 | 1.2E−08 | 0.08 | 6.5E−14 | 1 | 3 |
| rs2594935 | OCA2 | 15 | 25858633 | A | 0.21 | 1.1E−34 | −0.06 | 2.1E−06 | 1 | |
| rs728405 | OCA2 | 15 | 25873448 | C | 0.27 | 1.2E−42 | −0.07 | 4.1E−08 | 1 | |
| rs1800407 | OCA2 | 15 | 25903913 | T | 0.27 | 7.7E−13 | −0.29 | 1.7E−28 | 1 | 2 |
| rs3794604 | OCA2 | 15 | 25945660 | T | 0.40 | 2.5E−60 | 0.02 | 1.4E−01 | 0 | |
| rs4778232 | OCA2 | 15 | 25955360 | T | 0.30 | 2.9E−62 | −0.01 | 6.8E−01 | 1 | 11 |
| rs1448485 | OCA2 | 15 | 25956336 | T | 0.39 | 2.5E−68 | 0.01 | 6.6E−01 | 1 | |
| rs8024968 | OCA2 | 15 | 25957284 | T | 0.45 | 6.3E−74 | 0.03 | 1.3E−01 | 1 | 13 |
| rs1597196 | OCA2 | 15 | 25968517 | T | 0.33 | 5.4E−63 | 0.01 | 5.1E−01 | 1 | |
| rs7179994 | OCA2 | 15 | 25997365 | G | 0.31 | 2.0E−45 | −0.01 | 3.7E−01 | 1 | |
| rs4778138 | OCA2 | 15 | 26009415 | G | 0.73 | 4.7E−239 | 0.07 | 4.8E−05 | 1 | |
| rs4778241 | OCA2 | 15 | 26012308 | A | 0.75 | <1.0E−300 | −0.04 | 3.6E−02 | 1 | |
| rs7495174 | OCA2 | 15 | 26017833 | G | 1.05 | 4.7E−274 | 0.13 | 1.5E−07 | 1 | 8 |
| rs1129038 | HERC2 | 15 | 26030454 | C | 1.12 | <1.0E−300 | −0.03 | 8.6E−01 | 0 | |
| rs12593929 | HERC2 | 15 | 26032853 | G | 1.07 | 9.1E−265 | 0.11 | 1.8E−05 | 0 | |
| rs12913832 | HERC2 | 15 | 26039213 | A | 1.13 | <1.0E−300 | 1.13 | <1.0E−300 | 1 | 1 |
| rs7183877 | HERC2 | 15 | 26039328 | A | 0.89 | 5.7E−166 | −0.15 | 9.0E−09 | 1 | 10 |
| rs3935591 | HERC2 | 15 | 26047607 | T | 1.03 | <1.0E−300 | −0.04 | 7.6E−02 | 1 | |
| rs7170852 | HERC2 | 15 | 26101581 | T | 0.92 | <1.0E−300 | −0.01 | 7.2E−01 | 0 | |
| rs8041209 | HERC2 | 15 | 26117253 | T | 1.03 | 2.6E−226 | 0.09 | 5.7E−04 | 0 | |
| rs8028689 | HERC2 | 15 | 26162483 | C | 1.06 | 4.7E−236 | 0.09 | 7.0E−04 | 0 | |
| rs2240203 | HERC2 | 15 | 26167797 | C | 1.04 | 2.0E−230 | 0.09 | 9.1E−04 | 0 | |
| rs2240202 | HERC2 | 15 | 26184490 | A | 1.03 | 3.7E−221 | 0.09 | 8.6E−04 | 0 | |
| rs916977 | HERC2 | 15 | 26186959 | T | 1.05 | <1.0E−300 | −0.02 | 5.5E−01 | 0 | |
| rs16950979 | HERC2 | 15 | 26194101 | G | 1.05 | 2.8E−227 | 0.09 | 3.9E−04 | 0 | |
| rs2346050 | HERC2 | 15 | 26196279 | C | 1.04 | 2.0E−229 | 0.08 | 1.1E−03 | 0 | |
| rs16950987 | HERC2 | 15 | 26199823 | A | 1.05 | 2.1E−238 | 0.09 | 6.8E−04 | 0 | |
| rs1667394 | HERC2 | 15 | 26203777 | C | 1.06 | <1.0E−300 | 0.02 | 4.7E−01 | 1 | 9 |
| rs12592730 | HERC2 | 15 | 26203954 | A | 1.05 | 5.3E−223 | 0.09 | 5.1E−04 | 1 | 7 |
| rs1635168 | HERC2 | 15 | 26208861 | A | 1.03 | 3.0E−258 | 0.09 | 2.0E−04 | 0 | |
| rs6058017 | ASIP | 20 | 32320659 | G | −0.01 | 7.9E−01 | −0.02 | 2.7E−01 | 1 | 14 |

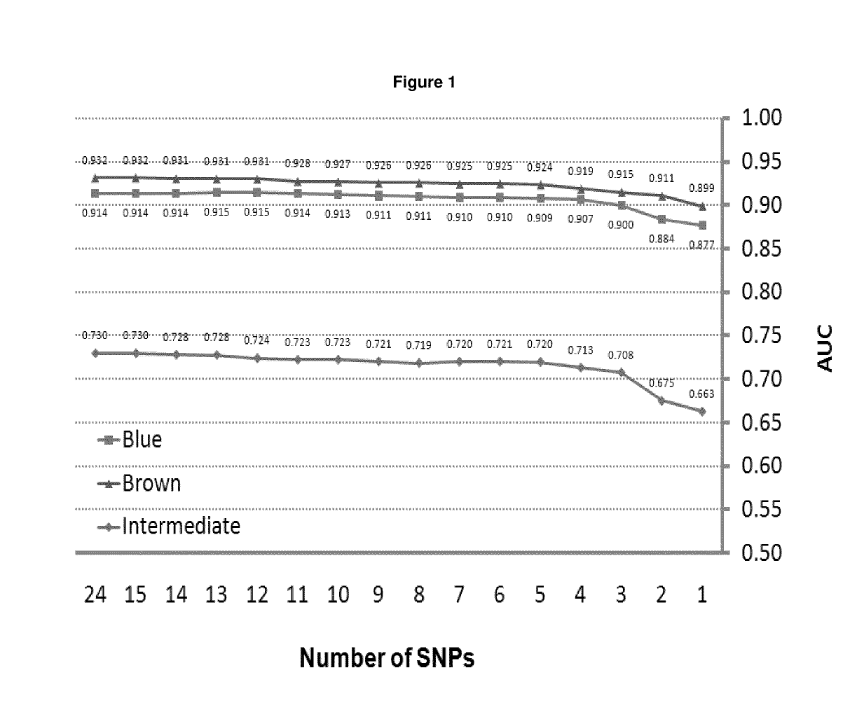

beta1, P1: betas and P-values derived from single SNP association tests unadjusted for rs12913832; beta2, P2: betas and P-values derived from single SNP association tests adjusted for rs12913832; P values smaller than 0.05 are indicated in bold; Tag: tagging SNPs were selected based on pair-wise r² < 0.8; Rank: 15 SNPs are ranked according to their contribution to eye color prediction when all 24 tagging SNPs were included in a multinomial logistic regression model, the smallest number represents highest prediction value, the 9 SNPs without number code did not contribute to the prediction accuracy, see main text and FIG. 1.

All SNPs genotyped were significantly associated (p<0.01) with eye color variation (Table 5), except one in the ASIP gene (but see below). A prediction model based on multinomial logistic regression constructed in the model-building set (N=3804, 61.7%) using 24 SNPs from eight genes (13 SNPs were removed because of strong LD with other markers in this set, Table 5) revealed excellent accuracy for predicting blue and brown eye color in the model-verification set (N=2364, 38.3%) based on five parameters (Table 6).

TABLE 6
DNA-based prediction of human eye (iris) color based on multinomial
logistic regression using 24 eye-color associated single nucleotide
polymorphisms in Dutch Europeans of the Rotterdam Study*

| | Blue | Intermediate | Brown |
|---|---|---|---|
| AUC | 0.91 | 0.73 | 0.93 |
| Sensitivity¹ | 93.4 | 1.1 | 88.4 |
| Specificity¹ | 77.1 | 99.6 | 88.0 |
| PPV¹ | 89.8 | 25.0 | 67.1 |
| NPV¹ | 84.4 | 90.0 | 96.5 |

¹Calculated from three 2 by 2 contingency tables of predicted and observed color types, where the predicted eye color type was obtained as the eye color with the highest predicted probability based on the multinomial logistic regression model,
AUC: Area Under the receiver operating characteristic (ROC) Curves,
PPV: Positive Predictive Value,
NPV: Negative Predictive Value,
*For results of four alternative prediction models, see Table 7.

Considering AUC as an overall measure for prediction accuracy, we obtained very high values for brown eyes at 0.93 and for blue eyes at 0.91. Note that a completely accurate prediction is obtained at an AUC of 1. The prediction of intermediate color was less accurate with an AUC of 0.73. Predicting eye color using four alternative models yielded similar results (Table 7). The lower prediction accuracy for intermediate color may be explained by unidentified associated SNPs and imprecise phenotype characterization; future investigations with more information on subtle phenotype characterization are warranted.

TABLE 7
Performances of four alternative models for DNA-based prediction of
human iris color using 24 associated single nucleotide polymorphisms
in Dutch Europeans of the Rotterdam Study*

| Model | Measure | Blue | Intermediate | Brown |
|---|---|---|---|---|
| Neural Network | Sensitivity | 92.9 | 0 | 91.7 |
| | Specificity | 79.4 | 100.0 | 87.0 |
| | PPV | 90.6 | —¹ | 66.3 |
| | NPV | 83.9 | 90.0 | 97.4 |
| | AUC | 0.89 | 0.65 | 0.91 |
| Fuzzy C-Means Clustering | Sensitivity | 93.0 | 0 | 85.2 |
| | Specificity | 75.8 | 100.0 | 86.8 |
| | PPV | 89.2 | —¹ | 64.3 |
| | NPV | 83.6 | 90.0 | 95.5 |
| | AUC | 0.91 | 0.67 | 0.93 |
| Ordinal Regression | Sensitivity | 93.5 | 0 | 88.5 |
| | Specificity | 77.0 | 100.0 | 87.7 |
| | PPV | 89.7 | —¹ | 66.6 |
| | NPV | 84.7 | 90.0 | 96.5 |
| | AUC | 0.91 | 0.73 | 0.93 |
| Classification Tree | Sensitivity | 91.5 | 13.0 | 74.9 |
| | Specificity | 75.3 | 95.0 | 90.3 |
| | PPV | 88.8 | 22.4 | 68.3 |
| | NPV | 80.6 | 90.7 | 92.8 |
| | AUC | —² | —² | —² |

*For results of the multinomial logistic regression model, see Table 6.
AUC: Area Under the receiver operating characteristic (ROC) Curves,
PPV: Positive Predictive Value,
NPV: Negative Predictive Value,
¹zero denominator,
²categorical outcomes were not measured by AUC.

Furthermore, to assess the contribution of each SNP to the prediction accuracy of eye color, we measured AUC in a step-wise manner by iteratively excluding one SNP from the multinomial logistic regression model. Six SNPs from six genes: HERC2 rs12913832, OCA2 rs1800407, SLC24A4 rs12896399, SLC45A2 rs16891982, TYR rs1393350, and IRF4 rs12203592 were revealed as major genetic eye color predictors with an overall AUC of 0.93 for brown, 0.91 for blue, and 0.72 for intermediate colored eyes (FIG. 1). Nine additional SNPs (from TYRP1, OCA2, HERC2, and ASIP, Table 5) had only minimal additive effects (FIG. 1). The remaining nine SNPs (Table 5) had no additive value to the predictive accuracy (FIG. 1); although they all were significantly associated with eye color in the single-SNP analysis (Table 5), their effects were most likely being covered by other markers from the same genes included in the set of 15 SNPs. The prediction accuracies presented here were improved considerably compared to our previous attempt using three SNPs in OCA2 and HERC2 (e.g. AUC=0.82 for brown eyes) [5], or compared to another prediction analysis [6] based on four SNPs in OCA2, HERC2, SLC24A4, and TYR that applied different methodology but estimating AUC in the Rotterdam Study using these four SNPs gave 0.83 for brown eyes.

The genetic prediction values obtained here for blue and brown eyes in Europeans represent the highest accuracies obtained so far in genetic prediction of human complex phenotypes. We thus demonstrated that accurate DNA-based prediction of complex human phenotypes is feasible if strong genetic variants are implicated. Our findings of statistically significant eye color association of several genes, together with the high prognostic value of SNPs therein, underline the importance of these genes in determining human iris color variation. Additionally, we provide a small set of DNA markers that are expected to serve as reliable biological evidence in suspect-less forensic cases potentially allowing the police to concentrate investigations for tracing unknown persons of European descent according to DNA-predicted eye color.

### REFERENCES

1. Janssens, A. C., and van Duijn, C. M. (2008). Genome-based prediction of common diseases: advances and prospects. Hum Mol Genet 17, R166-173.
2. Brand, A., Brand, H., and Schulte in den Baumen, T. (2008). The impact of genetics and genomics on public health. Eur J Hum Genet 16, 5-13.
3. Janssens, A. C., Gwinn, M., Bradley, L. A., Oostra, B. A., van Duijn, C. M., and Khoury, M. J. (2008). A critical appraisal of the scientific basis of commercial genomic profiles used to assess health risks and personalize health interventions. Am J Hum Genet 82, 593-599.
4. Haga, S. B., Khoury, M. J., and Burke, W. (2003). Genomic profiling to promote a healthy lifestyle: not ready for prime time. Nat Genet 34, 347-350.
5. Kayser, M., Liu, F., Janssens, A. C., Rivadeneira, F., Lao, O., van Duijn, K., Vermeulen, M., Arp, P., Jhamai, M. M., van Ijcken, W. F., et al. (2008). Three genome-wide association studies and a linkage analysis identify HERC2 as a human iris color gene. Am J Hum Genet 82, 411-423.
6. Sulem, P., Gudbjartsson, D. F., Stacey, S. N., Helgason, A., Rafnar, T., Magnusson, K. P., Manolescu, A., Karason, A., Palsson, A., Thorleifsson, G., et al. (2007). Genetic determinants of hair, eye and skin pigmentation in Europeans. Nat Genet 39, 1443-1452.
7. Sulem, P., Gudbjartsson, D. F., Stacey, S. N., Helgason, A., Rafnar, T., Jakobsdottir, M., Steinberg, S., Gudjonsson, S. A., Palsson, A., Thorleifsson, G., et al. (2008). Two newly identified genetic determinants of pigmentation in Europeans. Nat Genet 40, 835-837.
8. Sturm, R. A., Duffy, D. L., Zhao, Z. Z., Leite, F. P., Stark, M. S., Hayward, N. K., Martin, N. G., and Montgomery, G. W. (2008). A single SNP in an evolutionary conserved region within intron 86 of the HERC2 gene determines human blue-brown eye color. Am J Hum Genet 82, 424-431.
9. Eiberg, H., Troelsen, J., Nielsen, M., Mikkelsen, A., Mengel-From, J., Kjaer, K. W., and Hansen, L. (2008). Blue eye color in humans may be caused by a perfectly associated founder mutation in a regulatory element located within the HERC2 gene inhibiting OCA2 expression. Hum Genet 123, 177-187.
10. Han, J., Kraft, P., Nan, H., Guo, Q., Chen, C., Qureshi, A., Hankinson, S. E., Hu, F. B., Duffy, D. L., Zhao, Z. Z., et al. (2008). A genome-wide association study identifies novel alleles associated with hair color and skin pigmentation. PLoS Genet 4, e1000074.
11. Frudakis, T., Thomas, M., Gaskin, Z., Venkateswarlu, K., Chandra, K. S., Ginjupalli, S., Gunturi, S., Natrajan, S., Ponnuswamy, V. K., and Ponnuswamy, K. N. (2003). Sequences associated with human iris pigmentation. Genetics 165, 2071-2083.
12. Graf, J., Hodgson, R., and van Daal, A. (2005). Single nucleotide polymorphisms in the MATP gene are associated with normal human pigmentation variation. Hum Mutat 25, 278-284.
13. Kanetsky, P. A., Swoyer, J., Panossian, S., Holmes, R., Guerry, D., and Rebbeck, T. R. (2002). A polymorphism in the agouti signaling protein gene is associated with human pigmentation. Am J Hum Genet 70, 770-775.
14. Duffy, D. L., Montgomery, G. W., Chen, W., Zhao, Z. Z., Le, L., James, M. R., Hayward, N. K., Martin, N. G., and Sturm, R. A. (2007). A three-single-nucleotide polymorphism haplotype in intron 1 of OCA2 explains most human eye-color variation. Am J Hum Genet 80, 241-252.
15. Frudakis, T., Terravainen, T., and Thomas, M. (2007). Multilocus OCA2 genotypes specify human iris colors. Hum Genet 122, 311-326.
16. Hofman, A., Breteler, M. M., van Duijn, C. M., Krestin, G. P., Pols, H. A., Stricker, B. H., Tiemeier, H., Uitterlinden, A. G., Vingerling, J. R., and Witteman, J. C. (2007). The Rotterdam Study: objectives and design update. Eur J Epidemiol 22, 819-829.

### Supplemental Experimental Procedures

### Population Characteristics

The Rotterdam Study is a population-based prospective study of subjects aged 55 years or older [S1,S2]. Collection of eye (iris) color data and purification of DNA have been described in detail previously [S3]. In brief, each eye was examined by slit lamp examination by an ophthalmological medical researcher, iris color was graded by standard images showing various degrees of iris pigmentation and categorized into blue, brown and non-blue/non-brown called here intermediate. The Medical Ethics Committee of the Erasmus Medical Center approved the study protocol, and all participants provided written informed consent. Individuals identified as outliers using an identity-by-state analysis as described previously [S4] have been excluded because they most likely represent individuals of non-European ancestry.

### SNP Selection and Genotyping

We selected 37 SNPs that were statistically significantly associated with human iris color in previous studies [S3,S5-S13] (Table 8 and Table 4). Multiplex genotype assay design was performed with the software MassARRAY Assay Design version 3.1.2.2 (Sequenom Inc., San Diego, Calif.). We designed two 17-plex iPlex™ multiplexes, sequences of forward, reverse and extension primers are provided in Table 8. The Sequenom genotyping was performed on 5 ng of dried genomic DNA in 384-well plates (Applied Biosystems Inc. Foster City, Calif.) in a reaction volume of 5 μl containing 1×PCR Buffer, 1.625 mM MgCl, 2.5 μM dNTPs, 100 nM each PCR primer, 0.5 U PCR enzyme (Sequenom). The reaction was incubated in a GeneAmp PCR System 9700 (Applied Biosystems) at 94° C. for 4 minutes followed by 45 cycles of 94° C. for 20 seconds, 56° C. for 30 seconds, 72° C. for 1 minute, and finalized by 3 minutes at 72° C. To remove the excess dNTPs, 2 μl SAP mix containing 1×SAP Buffer and 0.5 U shrimp alkaline phosphatase (Sequenom) was added to the reaction. This was incubated in a GeneAmp PCR System 9700 (Applied Biosystems) at 37° C. for 40 minutes followed by 5 minutes at 85° C. for deactivation of the enzyme. Then 2 μl of Extension mix is added containing a concentration of adjusted extend primers varying between 3.5-7 μM for each primer, 1× iPLEX buffer (Sequenom), iPLEX termination mix (Sequenom) and iPLEX enzyme (Sequenom). The extension reaction was incubated in a GeneAmp PCR System 9700 (Applied Biosystems) at 94° C. for 30 seconds followed by 40 cycles of 94° C. for 5 seconds, 5 cycles of 52° C. for 5 seconds, and 80° C. for 5 seconds, and finalized at 72° C. for 3 minutes. After the extension reaction desaltation was carried out by adding 6 mg Clean Resin (Sequenom) and 16 μl water followed by rotating the plate for 15 minutes. The extension product was spotted onto a G384+10 SpectroCHIP (Sequenom) with the MassARRAY Nanodispenser model rs1000 (Sequenom). The chip was then transferred into the MassARRAY Compact System (Sequenom) where the data was collected, using TyperAnalyzer version 4.0.3.18 (Sequenom), SpectroACQUIRE version 3.3.1.3 (Sequenom), GenoFLEX version 1.1.79.0 (Sequenom) and MassArrayCALLER version 3.4.0.41 (Sequenom). For quality control reasons, the data was checked manually after data collection. In addition, rs6058017, was typed with the commercially available Taqman assay C_{—}22275334_{—}10 as recommended by the manufacturers (Applied Biosystems) and data for two other SNPs (rs12203592 and rs1408799) were used from microarray genotyping performed in the whole Rotterdam Study cohort using the Infinium II HumanHap550K Genotyping BeadChip® version 3 (IIlumina Inc. San Diego, Calif.) as described in detail previously [S4].

### Association and Linkage Disequilibrium Testing

Single SNP association was verified using a linear model where blue, intermediate, and brown were coded as 1, 2, and 3 quantitatively, and SNP genotypes were coded as 0, 1, or 2 minor alleles. Notably, rs12913832 in the HERC2 gene showed the largest effect (beta=1.13, P<1.0×10³⁰⁰), in agreement with previous findings [S7,S8]. Adjusting for the effect of rs12913832 led to multiple SNPs in the HERC2/OCA2 region becoming less or non-significant (Table 5), as expected due to the existing linkage disequilibrium (LD). However, rs1800407, a non-synonymous SNP in OCA2 (Arg419Gln), displayed considerably stronger significance after adjustment (P=1.7×10⁻²⁸ adjusted versus 7.7×10¹³ unadjusted), indicating an independent effect. Interestingly, this SNP was reported to act as a penetrance modifier of HERC2 rs12913832 [S7]. We performed a tagging SNP analysis excluding markers in strong LD (pair-wise r2>0.8) using software package Haploview 4.1 [S14]. Thirteen SNPs in strong LD were excluded from the OCA2-HERC2 region. Rs3794604 was excluded as being in strong LD with rs4778232, rs1448485, rs8024968 and rs1597196. Rs1129038, rs12593929, rs7170852, rs8041209, rs8028689, rs2240203, rs2240202, rs916977, rs16950979, rs2346050, rs16950987 and rs1635168 were excluded as being in strong LD with rs12913832, rs7183877, rs3935591, rs1667394 and rs12592730. Thus, a total of 24 SNPs were included in prediction analyses (Table 5).

### Prediction Modelling

The Rotterdam Study cohort was randomly split into a model-building set consisting of 3804 individuals and a model verification set consisting of the remaining 2364 individuals. Five models were constructed in the model-building set described in detail below.

### Ordinal Regression

Ordinal regression is often used when the response is categorical with ordered outcomes. The model provides predicted probabilities, inside the probability space, for each level of the response without assuming constant variance. Consider eye color, y, to be three ordinal levels “blue,” “intermediate”, and “brown”, which are determined by the genotype, x, of k SNPs. Let π1, π2, and π3 denote the probability of “blue,” “intermediate”, and “brown”, respectively. The ordinal regression can be written as

$$logit ( Pr ( y \leq blue \vert {x}_{1} … {x}_{k} ) ) = ln ( \frac{{π}_{1}}{1 - {π}_{1}} ) = {α}_{1} + \sum {β}_{k} {x}_{k}$$

$$logit ( Pr ( y \leq inter \vert {x}_{1} … {x}_{k} ) ) = ln ( \frac{{π}_{1} + {π}_{2}}{1 - ( {π}_{1} + {π}_{2} )} ) = {α}_{2} + \sum {β}_{k} {x}_{k} ,$$

where α and β can be derived in the model-building set.

Eye color of each individual in the model-verification set can be probabilistically predicted based on his or her genotypes and the derived α and β,

$${π}_{1} = \frac{exp ( {α}_{1} + \sum {β}_{k} {x}_{k} )}{1 + exp ( {α}_{1} + \sum {β}_{k} {x}_{k} )} , \\ {π}_{2} = \frac{exp ( {α}_{2} + \sum {β}_{k} {x}_{k} )}{1 + exp ( {α}_{2} + \sum {β}_{k} {x}_{k} )} {π}_{1} , and$$

$${π}_{3} = 1 - {π}_{1} - {π}_{2} .$$

### Multinomial Logistic Regression

Multinomial logistic regression is often used for categorical outcomes, where the model does not assume ordinary data, which can be written as:

$$logit ( Pr ( y = blue \vert {x}_{1} … {x}_{k} ) ) = ln ( \frac{{π}_{1}}{{π}_{3}} ) = {α}_{1} + \sum {β ( {π}_{1} )}_{k} {x}_{k}$$

$$logit ( Pr ( y = inter \vert {x}_{1} … {x}_{k} ) ) = ln ( \frac{{π}_{2}}{{π}_{3}} ) = {α}_{2} + \sum {β ( {π}_{2} )}_{k} {x}_{k} ,$$

and the probabilities for each individual being a certain color category can be estimated as:

$${π}_{1} = \frac{exp ( {α}_{1} + \sum {β ( {π}_{1} )}_{k} {x}_{k} )}{1 + exp ( {α}_{1} + \sum {β ( {π}_{1} )}_{k} {x}_{k} ) + exp ( {α}_{2} + \sum {β ( {π}_{2} )}_{k} {x}_{k} )}$$

$${π}_{2} = \frac{exp ( {α}_{2} + \sum {β ( {π}_{2} )}_{k} {x}_{k} )}{1 + exp ( {α}_{1} + \sum {β ( {π}_{1} )}_{k} {x}_{k} ) + exp ( {α}_{2} + \sum {β ( {π}_{2} )}_{k} {x}_{k} )} , and$$

$${π}_{3} = 1 - {π}_{1} - {π}_{2} .$$

For ordinal and multinomial logistic regressions, the color category with the max(π₁, π₂, π₃) was considered as the predicted color.

### Fuzzy C-Means Clustering (FCM)

There have been increasing interests in the methods based on machine-learning techniques, such as fuzzy logic and artificial neural networks. These methods can conveniently map an input space to an output space that is related through nonlinear functions which sometimes can be statistically complex. In this study we also constructed two prediction models based on fuzzy C-means clustering (FCM) and pattern-reorganization neural networks. FCM clustering is the most frequently used algorithm in generating a fuzzy inference system (FIS). It is based on iterative minimization of an objective function wherein each data point belongs to a cluster to some degree that is specified by a membership grade [S15]. A Sugeno-type FIS structure was generated based on FCM clustering in the model-building set. The input space was defined as a k-marker by N-individual matrix of the number of minor alleles plus one. The target variable was defined as a 3 by N matrix, where each row vector represents yes-no of the corresponding color type. The generated FIS was subsequently used to predict eye colors in the model-verification set, returning 3 membership grades of values between 0 and 1 for each individual indicating his or her color type. The color type with the maximal membership grade was considered as the predicted color.

### Neural Networks

Neural networks have been used to characterize gene-gene interactions [S16], find SNP-phenotype associations [S17,S18], and predict genetic phenotypes [S19]. A feed-forward network for pattern recognition was initialized in the model-building set, by specifying tan-sigmoid transfer functions in both the hidden and output layers. The hidden layer contained 10 arbitrary neurons and the output later contained three output neurons, each represents yes-no for one color type. The pattern recognition network was then trained using scaled conjugate gradient algorithm where the inputs and targets followed the same format described in the FCM section. During training, the model-building data set was randomly divided into three subsets, 60% were used for training, 20% were used to control for over-fitting by comparing the mean squared errors. The last 20% were used as an independent test of network generalization. The derived pattern recognition network was subsequently used to predict colors in the model-verification set, returning 3 numeric vectors with values between 0 and 1. The color type with the maximal value was considered as the predicted color.

### Classification Tree

Classification tree, one of the main data mining techniques, is used to predict membership of categorical objects from one or more predictors [S20]. Compared to multiple regression that simultaneously analyzes multiple predictors, the classification tree hierarchically and recursively conducts single regression analyses, where the next regression on a different predictor is conducted in the samples not classified in a previous regression. The assumptions regarding the level of measurement of predictors are less stringent compared to multiple regression. In the current study, the classification tree was trained in the model-building set and was used to predict eye colors in the model-verification set, returning an outcome with 3 categories representing each color.

### Model Evaluation

We evaluated the performance of the five prediction models in the model-verification set. A 2 by 2 confusion table was derived for each color type. The predicted color types were classified as true positives (TP), true negatives (TN), false positives (FP), or false negatives (FN). Four measurements of the prediction performance were derived:

Sensitivity=TP/(TP+FN)×100 is the percentage of correctly predicted color type among the observed color type. 1)

Specificity=TN/(TN+FP)×100 is the percentage of correctly predicted non-color type among the observed non-color type. 2)

Positive predictive value(PPV)=TP/(TP+FP)×100 is the percentage of correctly predicted color type among the predicted positives. 3)

Negative predictive value(NPV)=TN/(TN+FN)×100 is the percentage of correctly predicted non-color type among the predicted negatives. 4)

Additionally, we measured the area under the receiver operating characteristic (ROC) curves, or AUC [S21]. AUC is the integral of ROC curves which ranges from 0.5 representing total lack of prediction to 1.0 representing perfect prediction. AUC measures the predicted outcomes that are numeric or probabilistic values between 0 and 1. Because the classification tree gives categorical predictions or training frequencies that are non-accurate conditional probability estimates, the performance of classification tree was not evaluated using AUC. Because AUC is robust against the prevalence of each color type, we consider it as an overall measurement of model performance.

To access the contribution of each SNP to the predictive accuracy, we performed a step-wise analysis by iteratively excluding one SNP from the models. For each iteration, the lowest contributor in the model-building set was excluded; a model was then rebuilt; and subsequently used to re-predict colors in the model verification set. The contribution of each SNP was measured by the AUC loss of the models with and without that SNP.

Model building and verification procedures were programmed in MATLAB version 7.6.0 (The MathWorks, Inc., Natick, Mass.).

### SUPPLEMENTAL REFERENCES

S1. Hofman, A. et al (1991). Determinants of disease and disability in the elderly: the Rotterdam Elderly Study. Eur J Epidemiol 7, 403-422.
S2. Hofman, A. et al (2007). The Rotterdam Study: objectives and design update. Eur J Epidemiol 22, 819-829.
S3. Kayser, M. et al (2008). Three genome-wide association studies and a linkage analysis identify HERC2 as a human iris color gene. Am J Hum Genet 82, 411-423.
S4. Richards, J. B. et al (2008). Bone mineral density, osteoporosis, and osteoporotic fractures: a genome-wide association study. Lancet 371, 1505-1512.
S5. Sulem, P. et al (2008). Two newly identified genetic determinants of pigmentation in Europeans. Nat Genet 40, 835-837.
S6. Han, J. et al (2008). A genome-wide association study identifies novel alleles associated with hair color and skin pigmentation. PLoS Genet 4, e1000074.
S7. Sturm, R. A. et al (2008). A single SNP in an evolutionary conserved region within intron 86 of the HERC2 gene determines human blue-brown eye color. Am J Hum Genet 82, 424-431.
S8. Eiberg, H. et al (2008). Blue eye color in humans may be caused by a perfectly associated founder mutation in a regulatory element located within the HERC2 gene inhibiting OCA2 expression. Hum Genet 123, 177-187.
S9. Sulem, P. et al (2007). Genetic determinants of hair, eye and skin pigmentation in Europeans. Nat Genet 39, 1443-1452.
S10. Kanetsky, P. A. et al (2002). A polymorphism in the agouti signaling protein gene is associated with human pigmentation. Am J Hum Genet 70, 770-775.
S11. Duffy, D. L. et al (2007). A three-single-nucleotide polymorphism haplotype in intron 1 of OCA2 explains most human eye-color variation. Am J Hum Genet 80, 241-252.
S12. Graf, J. et al (2005). Single nucleotide polymorphisms in the MATP gene are associated with normal human pigmentation variation. Hum Mutat 25, 278-284.
S13. Frudakis, T. et al (2003). Sequences associated with human iris pigmentation. Genetics 165, 2071-2083.
S14. Barrett, J. C. et al (2005). Haploview: analysis and visualization of LD and haplotype maps. Bioinformatics 21, 263-265.
S15. Bezdek, J. C. (1981). Pattern Recognition with Fuzzy Objective Function Algorithms (New York: Kluwer Academic Publishers).
S16. Ritchie, M. D. et al (2003). Optimization of neural network architecture using genetic programming improves detection and modeling of gene-gene interactions in studies of human diseases. BMC Bioinformatics 4, 28.
S17. Moore, J. H. (2003). The ubiquitous nature of epistasis in determining susceptibility to common human diseases. Hum Hered 56, 73-82.
S18. North, B. V. et al (2003). Assessing optimal neural network architecture for identifying disease-associated multi-marker genotypes using a permutation test, and application to calpain 10 polymorphisms associated with diabetes. Ann Hum Genet 67, 348-356.
S19. Penco, S. et al (2008). New application of intelligent agents in sporadic amyotrophic lateral sclerosis identifies unexpected specific genetic background. BMC Bioinformatics 9, 254.
S20. Breiman, L. et al (1984). Classification and regression trees (Monterey, Calif., U.S.A.: Wadsworth, Inc.).
S21. Janssens, A. C. et al (2004). Revisiting the clinical validity of multiplex genetic testing in complex diseases. Am J Hum Genet 74, 585-588; author reply 588-589.

### Example 2

### IrisPlex™—a Sensitive DNA Tool for Accurate Prediction of Blue and Brown Eye Color in the Absence of Ancestry Information

### Abstract

A new era of ‘DNA intelligence’ is arriving in forensic biology, due to the impending ability to predict externally visible characteristics (EVCs) from biological material such as those found at crime scenes. EVC prediction from forensic samples, or from body parts, is expected to help concentrate police investigations towards finding unknown individuals, at times when conventional DNA profiling fails to provide informative leads. Here we present a robust and sensitive tool, termed IrisPlex™, for the accurate prediction of blue and brown eye color from DNA in future forensic applications. We used the 6 currently most eye color informative single nucleotide polymorphisms (SNPs) that previously revealed prevalence-adjusted prediction accuracies of over 90% for blue and brown eye color in 6168 Dutch Europeans. The single multiplex assay, based on SNaPshot chemistry and capillary electrophoresis, both widely used in forensic laboratories, displays high levels of genotyping sensitivity with complete profiles generated from as little as 31 pg of DNA, approximately 6 human diploid cell equivalents. We also present a prediction model to correctly classify an individual's eye color, via probability estimation solely based on DNA data, and illustrate the accuracy of the developed prediction test on 40 individuals from various geographic origins. Moreover, we obtained insights into the worldwide allele distribution of these 6 SNPs using the HGDP-CEPH samples of 51 populations. Eye color prediction analyses from HGDP-CEPH samples provide evidence that the test and model presented here perform reliably without prior ancestry information, although future worldwide genotype and phenotype data shall confirm this notion. As our IrisPlex™ eye color prediction test is capable of immediate implementation in forensic casework, it represents one of the first steps forward in the creation of a fully individualised EVC prediction system for future use in forensic DNA intelligence.

### Introduction

Predicting externally visible characteristics (EVCs) using informative molecular markers, such as those from DNA, has started to become a rapidly developing area in forensic genetics [1]. With knowledge gleaned from this type of data, it could be viewed as a ‘biological witness’ tool in suitable forensic cases, leading to a new era of ‘DNA intelligence’ (sometimes referred to as Forensic DNA Phenotyping); an era in which the externally visible traits of an individual may be defined solely from a biological sample left at a crime scene or from a dismemberment of a missing person. The most relevant forensic cases for DNA-based EVC prediction would be those in which the evidence DNA sample does not match either a suspect's conventional short tandem repeat (STR) profile or any from a criminal DNA database, and also where no additional knowledge about the sample donor exists. DNA-based EVC prediction is also suitable in cases where eye witnesses are available, but their statements about the appearance of an unknown suspect may wish to be confirmed before use in intelligence work. Furthermore, in disaster victim identification or other cases of missing person identification, DNA-based EVC prediction would be useful whenever conventional STR profiles obtained do not match any putatively related individual. Unfortunately, at present, the molecular genetics of individual-specific EVCs remains largely unknown, with little expectation for immediate forensic application. However, a number of group-specific EVCs, such as eye color, are being understood more and more in their genetic determination [2-9] and models for predicting phenotypes solely based on genotypes are being developed [10] with great promise for forensic applications. In certain cases, for example, if the police have no evidence on where/how to find a crime scene sample donor, or how to reveal the identity of a missing person, group-specific EVCs are already expected to be useful for tracing unknown individuals by focusing intelligence work on the most likely appearance group to which the individual in question belongs [1].

Human eye (iris) color is a highly polymorphic phenotype in people of European descent and, albeit less so in those from surrounding regions such as the Middle East or Western Asia [11], and is under strong genetic control [12]. Brown eye color is assumed to reflect the ancestral human state [4] and is present everywhere in the world including Europe, although in lower frequencies, especially in its northern parts. Non-brown eye colors are assumed to be of European origin and to have been driven by positive selection starting in early European history, perhaps as a result of rare color preferences in human mate choice [3, 13]. Recent years have yielded intensive studies to increase the genetic understanding of human eye color, via genome-wide association and linkage analysis or candidate gene studies [2-8, 14-17]. The OCA2 gene on chromosome 15 was originally thought to be the most informative human eye color gene due to its association with the human P protein required for the processing of melanosomal proteins [9], and mutations in this gene do result in pigmentation disorders [18]. However, recent studies have shown that genetic variants in the neighbouring HERC2 gene are more significantly associated with eye color variation than those in OCA2 [2-6]. Also, one of the most significant non-synonymous SNPs associated with eye color, rs1800407 located in exon 12 of the OCA2 gene, acts only as a penetrance modifier of rs12913832 in HERC2 and is, to a lesser extent, independently associated with eye color variation [5]. It is currently assumed that genetic variation in HERC2 acts as a functional regulator of adjacent OCA2 gene activity [3-5, 19], although more work is needed to fully establish the functional relationship between these two genes. While the HERC2/OCA2 region harbours most blue and brown eye color information, other genes were also identified as contributing to eye color variation, such as SLC24A4, SLC45A2 (MATP), TYRP1, TYR, ASIP and IRF4, although to a much lesser degree [2, 6-8, 17]. A recent study on 6168 Dutch Europeans demonstrated that with 15 eye-color associated SNPs from 8 genes, blue and brown eye colors can be predicted with >90% prevalence-adjusted accuracy and that most eye color information is provided by a subset of just 6 SNPs from 6 genes [10].

Many of the currently known eye color-associated SNPs, including those with high prediction value, are located in introns without functional evidence for causal trait involvement. They most likely provide eye color information due to physical linkage with causal but currently unknown variants. This is because commercially available SNP microarrays, used in genome-wide association studies of complex traits including eye color, are strongly biased towards non-coding markers. Due to the assumed positive selection history of non-brown eye color in Europe, it can be expected that non-causal alleles, with association to non-brown eye color in people of European (and neighbouring) ancestry, also exist in individuals of different ancestries that lack non-brown colored eyes, which may result in wrong prediction outcomes. Indeed, an inspection of eye-color associated SNPs in the limited non-European data of the International HapMap Project revealed small to considerable frequencies of blue-eye associated homozygote alleles, although blue-eyed individuals are very unlikely to occur in these East Asian and African populations. Examples are the CC/GG allele of rs916977 in the HERC2 gene observed in 2/90 HapMap East Asians, or the TT/AA allele of rs4778138 and rs7495174, both in the OCA2 gene, found in 5/90 and 7/90 HapMap East Asians, as well as in 3/60 and 43/60 HapMap Africans, respectively [4]. However, more detailed worldwide data are needed to assess whether DNA-based eye color prediction only works reliably when the geographic origin of the person in question is known e.g. from additional DNA-based ancestry testing.

Here, we have developed a single multiplex genotyping system, termed IrisPlex™, for the 6 currently most eye color-informative SNPs to accurately predict human blue and brown eye color. To allow future forensic applications, we focussed on a technical platform widely applied by forensic laboratories, and investigated its degree of sensitivity. We include the prediction model which can correctly classify an individual's eye color solely based on DNA data and illustrate the accuracy of the developed prediction system on individuals from various geographic origins. Furthermore, we applied the IrisPlex™ tool to the HGDP-CEPH samples representing 51 worldwide populations, and performed model-based eye color prediction on a worldwide scale.

### Materials & Methods

### Sample Collection & Iris Photography

Buccal swabs were taken from 40 volunteers with informed consent. A photographic image of their iris was taken concurrently with a macro lens, ensuring that similar distance and light conditions were used for each photo for normalisation. Information regarding the sex and country of birth for each individual was also collected. DNA was extracted using the QIAamp DNA Mini kit according to the manufacturer's protocol (Qiagen, Hagen, Germany). We also obtained the H952 subset of the HGDP-CEPH samples representing 952 individuals from 51 worldwide populations [20, 21]. This subset excludes duplicates, mix-ups and relatives up to the level of first-degree cousins. Due to the lack of DNA, 18 samples could not be genotyped for all markers, leaving a total of 934 worldwide HGDP samples in this study.

### Multiplex Design, Genotyping and Sensitivity Testing

Six SNPs; rs12913832, rs1800407, rs12896399, rs16891982, rs1393350 and rs12203592 from the HERC2, OCA2, SLC24A4, SLC45A2 (MATP), TYR and IRF4 genes respectively, were used in this study. The six PCR primer pairs were designed using the free web-based design software Primer3Plus [22] using the default parameters of the program. Each PCR fragment size was limited to less than 150 bp to cater for degraded DNA samples, vital for future application on forensic samples. The sequences surrounding the relevant SNP were searched with BLAST [23] against dbSNP [24] for other SNP sites that may interfere with primer binding, and these sites were avoided. Also, to ensure there would be little interaction between all six forward and reverse primers, the software program AutoDimer [25] was used throughout the design. The PCR primer sequences can be found in Table 8. For the single multiplex PCR, a total of 1 μl (0.5-2 ng) genomic DNA extract from each individual was amplified in a 12 μl PCR reaction with 1×PCR buffer, 2.7 mM MgCl2, 200 μM of each dNTP, primer concentrations of 0.416 μM each and 0.5 U AmpliTaq Gold DNA polymerase (Applied Biosystems Inc., Foster City, Calif.). Thermal cycling for PCR was performed on the gold-plated 96-well GeneAmp® PCR system 9700 (Applied Biosystems). The conditions for multiplex PCR were as follows: (1) 95° C. for 10 min, (2) 33 cycles of 95° C. for 30 s and 60° C. for 30 s, (3) 5 min at 60° C. Both forward and reverse SBE primers were designed for each SNP and the six final primers chosen were based on their suitability for the multiplex and the genotype of the resultant product to allow complete multiplexing. The primer sequences and specifications can be found in Table 9 and Table 10. The design followed a similar protocol to the PCR primer design ensuring primer melting temperatures of approximately 55° C. for the SBE reaction and all possible primer interactions were screened. To ensure complete capillary separation between the products, poly-T tails of varying sizes were added to the 5′ ends of the six SBE primers. Following PCR product purification to remove unincorporated primers and dNTPs, the multiplex SBE assay was performed using 1 μl of product with 1 μl SNaPshot reaction mix in a total reaction volume of 5 μl. Thermal cycling for SBE was performed on the gold-plated 96-well GeneAmp® PCR system 9700 (Applied Biosystems). The following thermocycling programme was used: 96° C. for 2 min and 25 cycles of 96° C. for 10 s, 50° C. for 5 s and 60° C. for 30 s. Excess fluorescently labelled ddNTPs were inactivated and 1 μl of cleaned multiplex extension products were then run on an ABI 3130xl Genetic Analyser (Applied Biosystems) following the ABI Prism® SNaPshot kit standard protocol (Applied Biosystems). Allele calling was performed using GeneMapper v. 3.7 software (Applied Biosystems). A custom designed bin set was implemented to allow automation of genotyping. For sensitivity testing, a threshold of 50 rfu for peak intensities was adopted to ensure accuracy of genotyping. Samples from three different individuals (brown, intermediate and blue eye color) were measured and quantified in a dilution series using the Quantifiler™ Human DNA Quantification kit (Applied Biosystems). Template concentrations from 0.5 ng/μl-0.015 ng/μl were also run in duplicate to test the overall sensitivity of the multiplex.

### Statistical Analysis

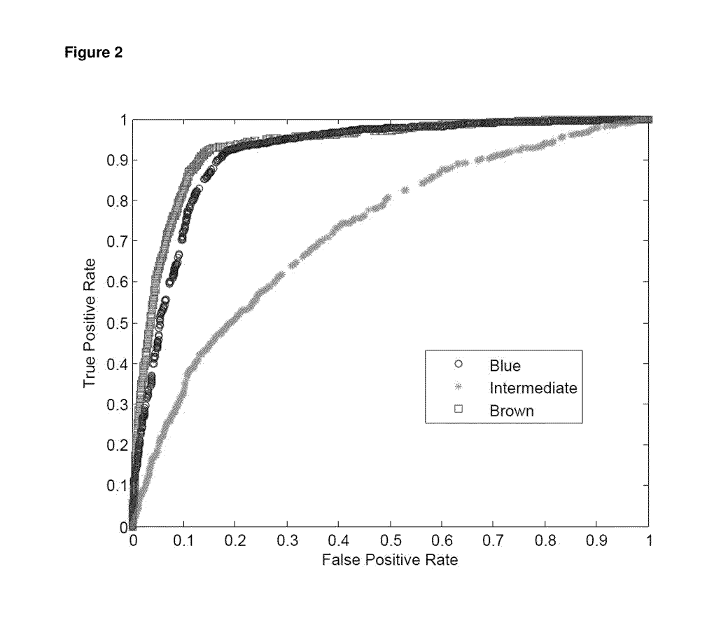

Liu et al. [10] have previously published the formula used in this study for eye color prediction. It is based on a multinomial logistic regression model. The probabilities of each individual being brown (π1), blue (π2), and otherwise (π3) were calculated based on the sample genotypes,

$${π}_{1} = \frac{exp ( {α}_{1} + \sum {β ( {π}_{1} )}_{k} {x}_{k} )}{1 + exp ( {α}_{1} + \sum {β ( {π}_{1} )}_{k} {x}_{k} ) + exp ( {α}_{2} + \sum {β ( {π}_{2} )}_{k} {x}_{k} )}$$

$${π}_{2} = \frac{exp ( {α}_{2} + \sum {β ( {π}_{2} )}_{k} {x}_{k} )}{1 + exp ( {α}_{1} + \sum {β ( {π}_{1} )}_{k} {x}_{k} ) + exp ( {α}_{2} + \sum {β ( {π}_{2} )}_{k} {x}_{k} )} , and$$

$${π}_{3} = 1 - {π}_{1} - {π}_{2} .$$

where xk is the number of minor alleles of the kth SNP. The model parameters, alpha and beta were derived based on 3804 Dutch individuals in the model-building set of the previous study [10] and can be found in Table 11. These probabilities can be calculated using a macro. Each individual is classified as being brown, blue or intermediate based on the predicted probabilities derived from the above formula. For example, a phenotypic brown-eyed individual can give a probability value of 0.76 for brown, 0.09 for blue and 0.15 for intermediate. For the worldwide distribution, a threshold of 0.7 predicted eye color probability was used for categorisation. For example, an individual is predicted as brown if π3>0.7, otherwise they are predicted as undefined. This cut off was chosen based on the receiver operating characteristic (ROC) curve derived from the Dutch study [10], where after the false positive rate of 0.3 (corresponding to specificity of 0.7), the decrease of the true positive rate becomes costly, with the possibility of errors increasing, as seen in FIG. 2 (see below for a discussion on the selection of the appropriate threshold). To evaluate the prediction accuracy on the worldwide samples, we assumed that all individuals outside of Europe and Western Russia are brown eyed, as phenotypic data are not available for the HGDP-CEPH individuals. MapViewer 7 (Golden Software, Inc., Golden, Colo., USA) package was used to plot the distribution of SNP genotypes and the predicted eye color, on the world map. A non-metric multi-dimensional scaling (MDS) plot was produced to illustrate the pair-wise FST distances [26] of the 6 eye color SNPs between populations, using SPSS 15.0.1 for Windows (SPSS Inc., Chicago, USA). Analysis of MOlecular VAriance (AMOVA) [27] was performed using ARLEQUIN v3.11 [28].

### Results & Discussion

### IrisPlex™ Design & Sensitivity

The design of the IrisPlex™ assay considered fragment lengths of only 80 to 128 bp, allowing future application to forensic samples that often contain fragmented DNA due to degradation. It was also designed so that extension products were evenly separated by 6 bp in the region of 30-65 bp in length to ensure unequivocal marker differentiation. PCR and SBE multiplex optimisations aimed to balance all SNP alleles, generating similar peak intensities to ensure genotyping accuracy in a wide range of DNA quantities. However, despite extensive efforts, allele balance was not completely achieved e.g. allele T of rs12896399 in its heterozygote state, or allele C of rs16891982 in its homozygote state were lower in comparison. Nevertheless, this slight imbalance does not affect the genotyping accuracy, unless the DNA quantity falls below the sensitivity threshold, and thus appeared sufficient for practical applications. The assay works optimally between 0.25-0.5 ng of template DNA, but also reveals complete 6-SNP profiles down to a level of 31 pg representing approximately 6 human diploid cells. Only at 15 pg of DNA template were allelic drop-outs observed for some of the SNPs. Notably, the sensitivity achieved was considerably higher than those previously reported for autosomal SNP multiplexes introduced for human identification purposes (which may be influenced by SNP numbers included). For example, 500 pg of DNA was required for a full profile of 52 SNPs analysed in two SBE multiplexes after a single multiplex PCR [29], and also for a full 20 SNP (plus amelogenin) profile from a single tube PCR reaction [30]. The sensitivity of the IrisPlex™ system is also considerably higher than that of the commercially available AmpFISTR Minifiler kit (Applied Biosystems) recommended for degraded DNA typing, which requires at least 125 pg of input DNA for full profiles of 8 autosomal STRs (plus amelogenin) [31]. We therefore expect the sensitivity of our IrisPlex™ system to meet the requirements of routine forensic applications in most cases, with it expected to be more successful than multiplex SNP/STR systems currently used in forensic practice.

### Prediction Probability Accuracy

We previously established in a large set of 6168 Dutch Europeans that the 6 SNPs from 6 genes included in this multiplex assay carry the most eye color prediction information from all currently known eye color associated SNPs [10; and Example 1]. Considering the area under the receiver characteristic operating curves (AUC) as an overall measure for prevalence-adjusted prediction accuracy, whereby a completely accurate prediction is obtained at an AUC of 1 and random prediction at 0.5, very high values for brown eyes at 0.93 and for blue eyes at 0.91 were obtained [10]. To further illustrate the predictive performance of the IrisPlex™ and to demonstrate the system's reliability, we generated data on 40 individuals from various geographic origins (Table 12). The individuals are ordered based on eye color prediction probabilities, starting with highest probability of blue, then highest probability of intermediate, and ending with highest probability of brown. The prediction probabilities for all three eye color categories are provided for each sample. The actual eye color is also indicated for each individual. It is evident that there is a clear correlation between the predicted values and the actual eye color phenotypes, thus confirming the accuracy of the 6 SNP prediction model. For 37 (92.5%) of the individuals the genetic eye color prediction perfectly agreed with the eye color phenotype from visual inspection (Fisher's exact test p value=9.78×10-9). Only three individuals were incorrectly categorised into their brown/blue categories by the prediction model, or were inconclusive (see actual eye colors and predictions marked in bold in Table 12). From this 40 person data set, the correct call rate (sensitivity) of the model when using an accuracy of above 0.7 was 91.6% for blue eye color categorisation and 56% for brown. However, as can be seen from the examples (albeit limited) in Table 12, individuals with prediction probabilities for brown between 0.5 and 0.7 also have brown eyes. Lowering the eye color probability threshold to 0.5 resulted in 87.5% correct brown eye color categorisation, while the 91.6% for blue remained the same. The 0.5 probability level successfully illustrates the sensitivity of the model in comparison to established data from the previously published Dutch European cohort, where sensitivity values of 88.4% for brown and 93.4% for blue eye color characterisation were achieved [10]. Altering the probability level can achieve higher specificity levels, although this will affect the overall sensitivity of the model. For example, probability levels of 0.9 and above will increase the specificity dramatically for true blue and true brown homozygotes, with 24 out of the 40 individuals showing 100% prediction accuracy. However, using such a high threshold, light and dark intermediates that could be visually viewed as slight variations of blue or brown, respectively, would then fail to be categorised into blue or brown. So far, these “intermediate” eye colors are more challenging to define using the present prediction model and the currently-available SNPs. Notably, in our previous study involving several thousand Dutch Europeans, we observed that at the 0.5 threshold, the prediction accuracy for intermediate (i.e. non-blue/non-brown) eye colors was considerably lower at only 0.73 than that seen for blue and brown colors at >0.91 [10]. We hypothesised that the lower prediction accuracy reached for these intermediate colors may be explained by imprecise phenotype categorisation or the result of unidentified genetic determinants [10]. Hence, more work is needed to find genetic variants with high predictive value for the non-blue and non-brown eye colors. Finally, discrepancies between genetically predicted and true phenotypic eye color may be caused by the fact that eye color can change over one's lifetime. However, as this is a rare phenomenon [32], it is not expected to affect our prediction test significantly, but may be a contributing factor as to why we could not assign three test individuals in this study correctly, as well as deviations from 100% prediction accuracy in our previous study [10].

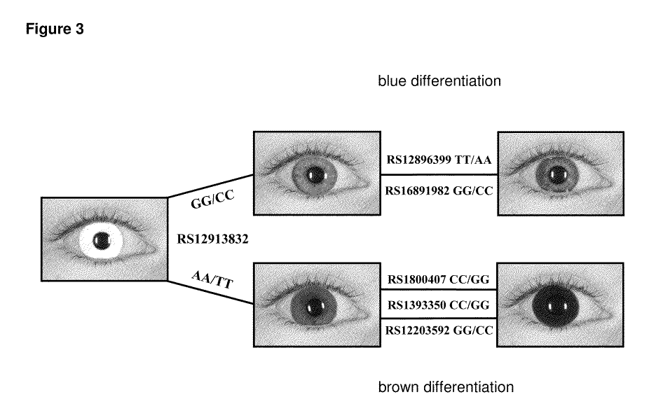

Each of the 6 SNPs included in the IrisPlex™ system provides mounting genetic information towards the overall prediction accuracy achievable with this DNA test system, although with different input. As previously established [10], rs12913832 in the HERC2 gene alone carries most of the eye color predictive information with an AUC of 0.899 for brown and 0.877 for blue achieved with this single SNP. This is in line with previous association studies showing that this SNP is the most strongly eye color associated SNP currently known [3, 5, 10]. The additional 5 SNPs from the additional 5 genes OCA2, SLC24A4, SLC45A2 (MATP), TYR and IRF4 included in the assay slightly increased the prediction accuracy as reflected in the prediction rank established previously [10] due to their lower (but still significant) eye color association as established previously [2, 6, 7, 10]. Notably, two of them, rs1393350 from the TYR gene and rs12896399 from the SLC24A4 gene, reached much lower P-values for association when comparing individuals with blue versus green eyes relative to blue versus brown eyes previously [2]. This may indicate that they contribute more to the blue and intermediate prediction and less to the brown prediction. The P-values and adjusted rs12913832 beta values for the 6 SNPs involved in this prediction model can be found in the supplementary material of Liu et al. [10] as the 6 highest ranking SNPs. In general, to understand the impact of each SNP on the prediction model, two scenarios have been displayed in FIG. 3 for the genetic variation in eye color based on the 6 SNPs presented. As depicted, the major impact in determining whether the eye color will be brown versus non-brown comes from rs12913832 (HERC2) with its AA/TT vs. GG/CC homozygote genotypes. Further determination of non-brown is provided by rs12896399 (SLC24A4) and its TT/AA, as well as by rs16891982 (SLC45A2 (MATP)) and its GG/CC homozygote genotype. On the other hand, further darkening of brown is determined by the homozygote genotype CC/GG of rs1800407 (OCA2) and rs1393350 (TYR), respectively, as well as the GG/CC homozygote genotype of rs12203592 (IRF4).

### Genetic Diversity and Eye Color Prediction on a Worldwide Scale

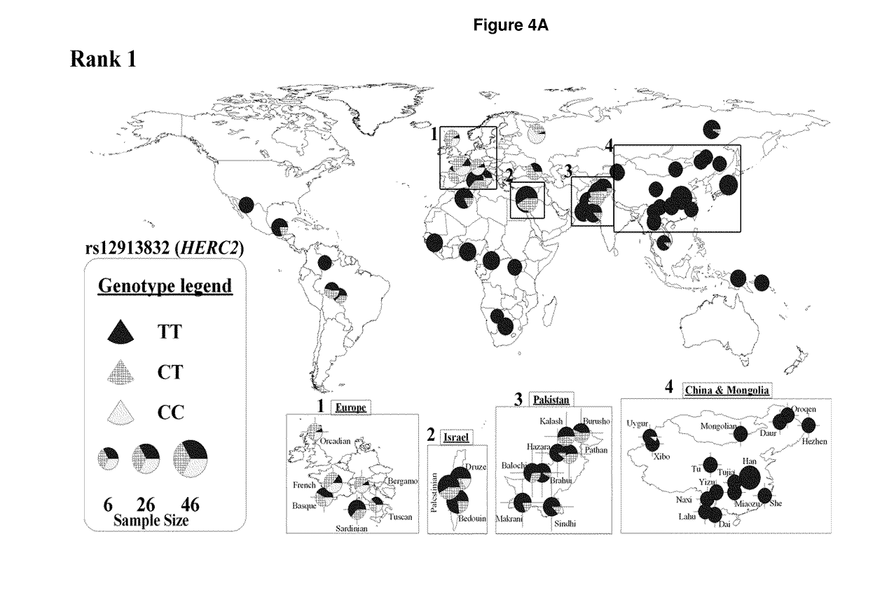

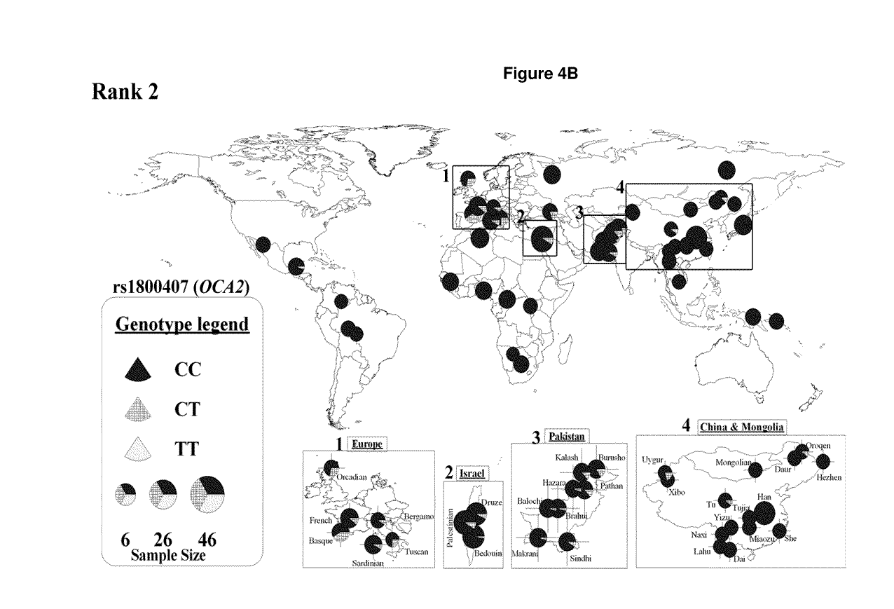

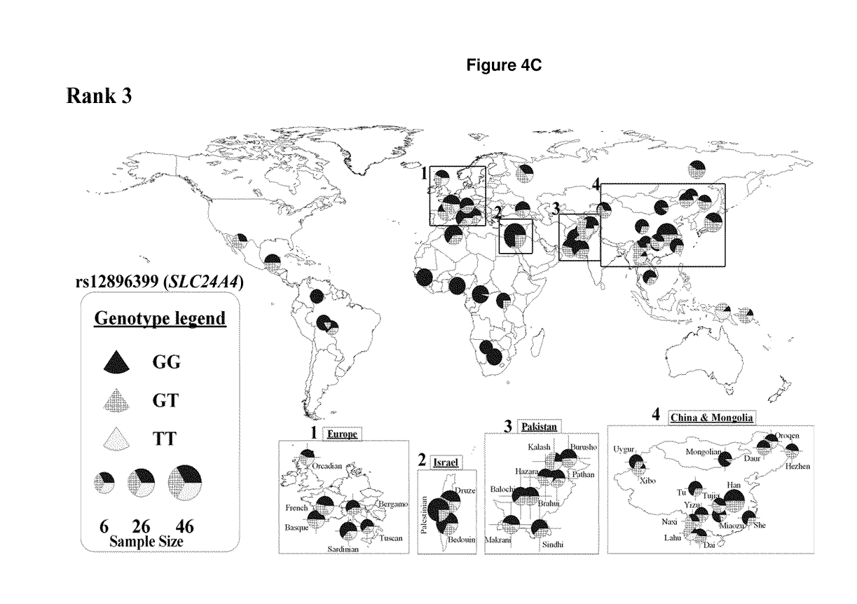

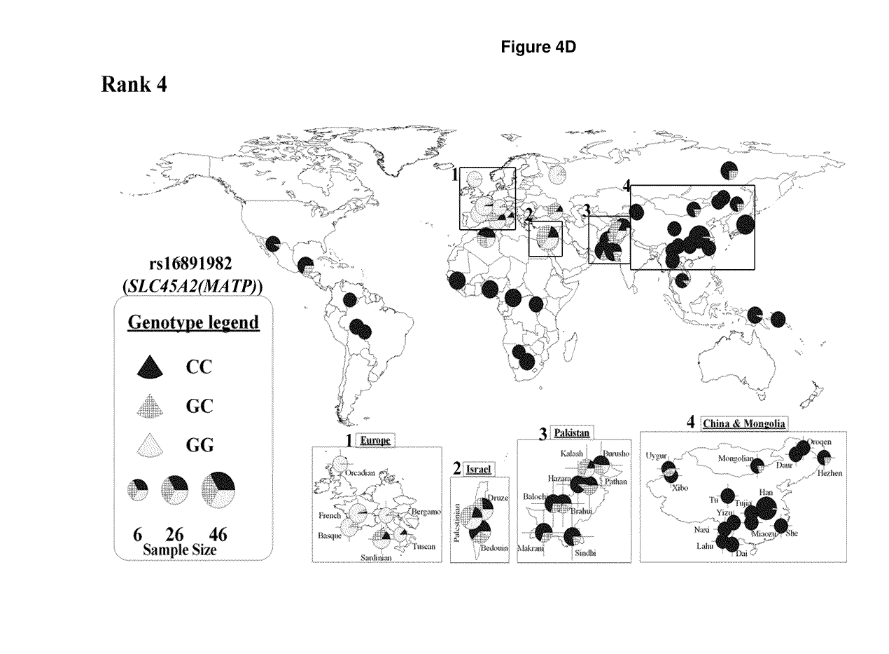

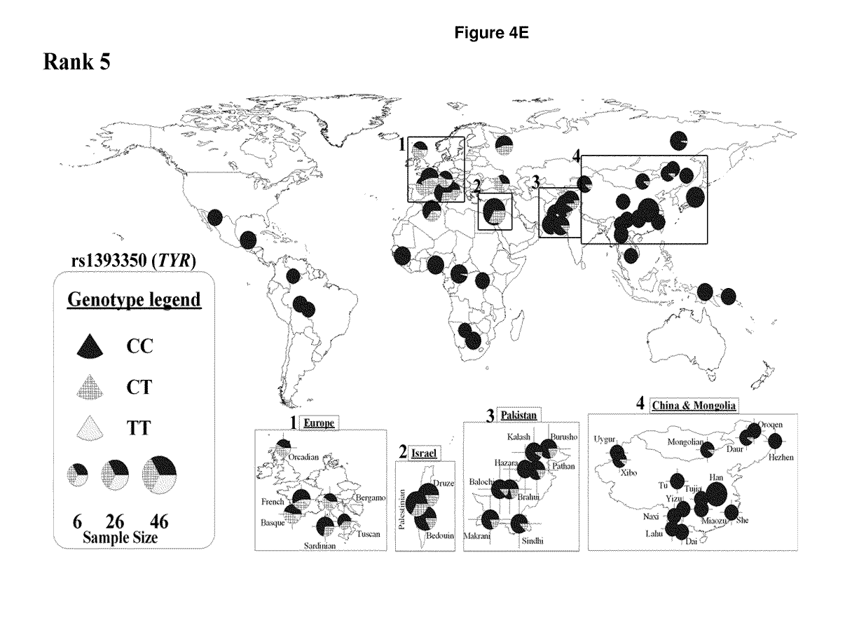

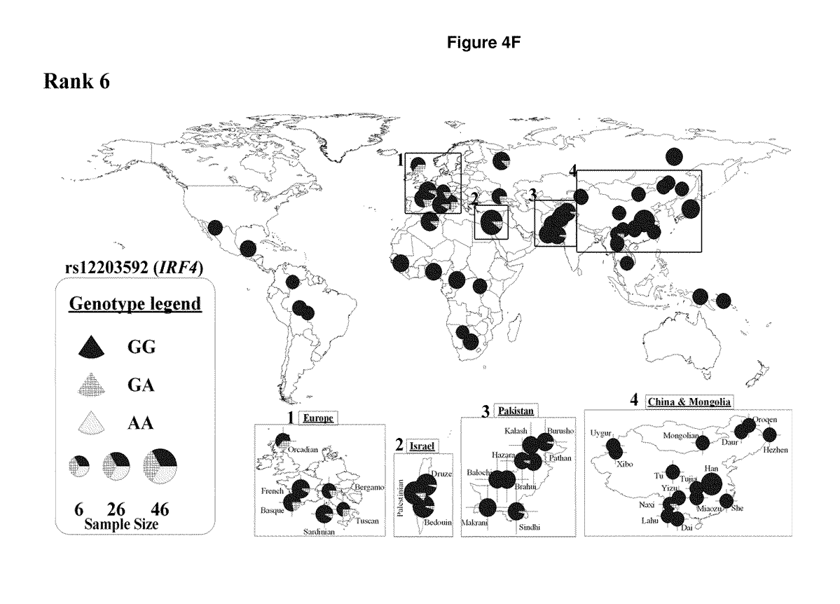

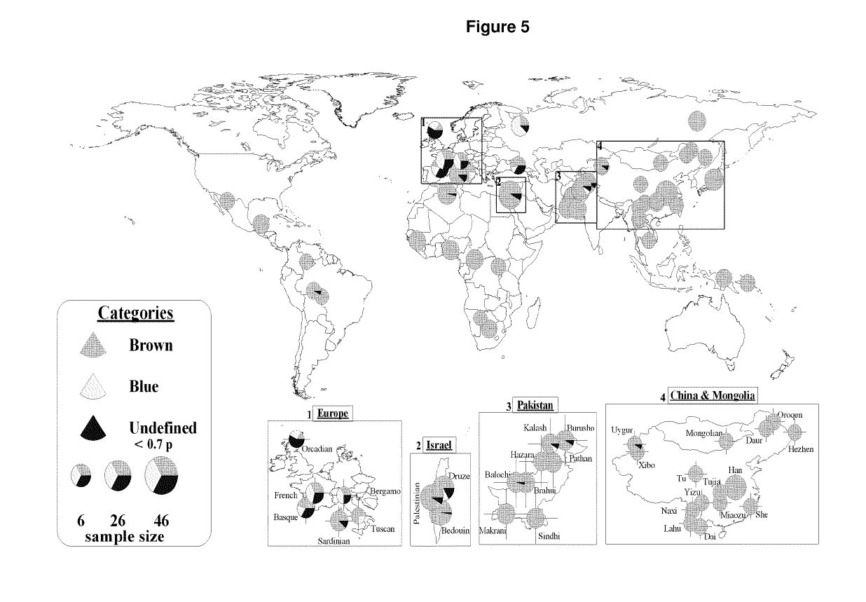

FIG. 4 displays the genotypes of 934 individuals from 51 HGDP-CEPH populations for each of the 6 SNPs included in the multiplex prediction test. FIG. 5a presents the most eye-color associated and the highest prediction-ranking rs12913832 (HERC2) SNP. It is apparent that the blue-eye associated homozygote genotype (CC/GG) as well as the heterozygote genotype, are both almost exclusively restricted to Europe and the surrounding areas such as the Middle East and West Asia, where blue and intermediate eye colors are expected. On the other hand, the brown-eye associated homozygote genotype (TT/AA) of this SNP exists everywhere in the world and is (almost) the only genotype found in areas such as East Asia, Oceania and Sub-Saharan Africa where only brown eye color is expected. Our HGDP data on the Japanese population confirm a recent study, which demonstrated that all the Japanese individuals involved had brown eyes and carried the TT/AA genotype [33]. The geographic pattern observed for rs12913832 (HERC2) is also evident for rs16891982 (SLC45A2 (MATP)), ranked number 4 in the Dutch cohort prediction [10]. However, unlike rs12913832 (HERC2), the blue-eye associated homozygote genotype of rs16891982 (SLC45A2 (MATP)) is of much higher frequency in Europe, Middle East and West Asia. Rs1800407 (OCA2), ranked number 2 [10] (FIG. 4b), is postulated to act as a penetrance modifier on rs12913832 (HERC2), and is less defined in its homozygote blue eye genotype, which is very rare, but does display the heterozygous genotype to a considerable degree within Europe, with minor frequency in the Middle East and West Asia and quite uncommon in the rest of the world. Similar findings are obtained for rs1393350 (TYR), ranked number [10] (FIG. 4e) and rs12203592 (IRF4) ranked number 6 in the large Dutch population study [10] (FIG. 4f). Rs12896399 (SLC24A4) displays no recognisable trend in the geographic distribution of the two alleles (FIG. 4c), which is remarkable as this SNP was ranked third best in the Dutch cohort [10]. Hence, apart from rs12896399, there is an increase in frequency of the blue-eye associated homozygote and the heterozygote genotypes towards Europe and, albeit less so in the Middle East and West Asia, which corroborates the degree of expected eye color variation in these regions. Conversely, brown-eye associated homozygote genotypes are predominant in East Asia, Sub-Saharan Africa, Native America and Oceania in agreement with the expected monomorphy of eye color (brown) in these areas.

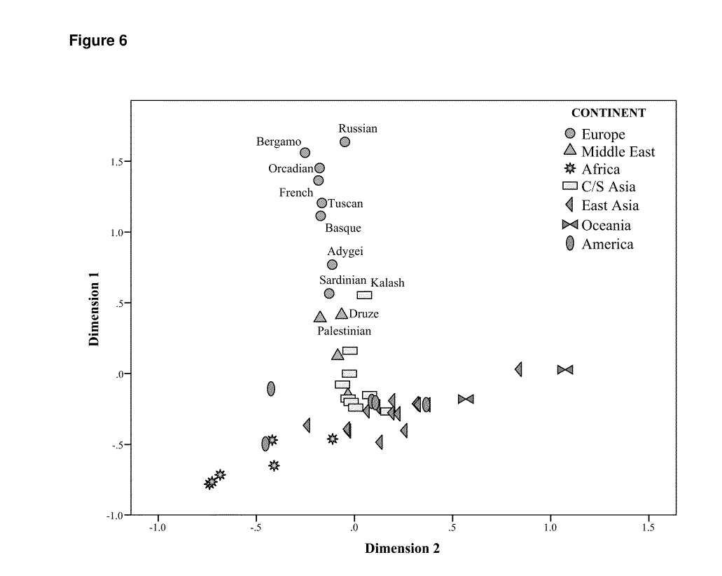

The variation in worldwide allele distributions between these 6 SNPs underlines the importance of using a combined SNP model to accurately predict eye color. FIG. 6 is an illustration of the predicted eye colors of the HGDP-CEPH worldwide panel using this model, in which a probability threshold of 0.7 was applied. The results clearly demonstrate that blue eye color is only predicted in Europe, and, albeit more rarely so in the Middle East and West Asia, but never elsewhere in the world. In particular, blue eye color was predicted in Europeans (including Western Russians) with an average probability of 0.86. Similarly, individuals with predicted non-blue and non-brown colors, which are included in the prediction group below the probability threshold, are mostly observed in Europe and, albeit less so in the Middle East and West Asia, but never elsewhere in the world (with the exception of a single individual from Brazil with a brown probability of 0.48 and two from Algeria with brown probabilities just short of the 0.7 threshold at 0.69). Moreover, brown eye color is predicted everywhere in the world but is the only predicted eye color in East Asia, Oceania, Sub-Saharan Africa and Native America (with the noted single exception). In particular, brown eye color in the HGDP samples from outside Europe, Middle East and West Asia, i.e. in regions where only brown eyes are expected, was predicted with an average probability of 0.997. Unfortunately, there are no individual eye color phenotypes available for the HGDP-CEPH samples; but our DNA-predicted eye color results are in agreement with general knowledge and reported data [11] on the distribution of eye color phenotypic variation around the world. However, without eye color phenotype information we cannot exclude for certain the existence of non-brown eye color outside Europe that remains undetectable via the SNPs used here (which were all identified in previous association studies on European populations). Nonetheless, we regard this scenario as highly unlikely given the assumed European origin of non-brown eye color variation [13]. For additional confirmation of the reliable worldwide use of our eye color prediction test without prior ancestry information, we would also like to emphasise that the brown eye color of all of the individuals from the 40-person illustration dataset whose country of origin is outside of Europe were predicted correctly (Table 12).

Ancestry Inference with Eye Color SNPs

A notion that has been advocated in the past, is inferring biogeographic ancestry (or genetic origins) from DNA markers derived from pigmentation genes [34]. We tested the power of the 6 eye color predictive SNPs for differentiating worldwide human populations and individuals. First, we asked by means of an AMOVA test how much of the total genetic variation provided by the 6 SNPs is explained by geography with assigning the 51 HGDP-CEPH populations to 7 regional geographic groups. A variance proportion of 24.1% was estimated from 10100 permutations, which was statistically significant (P<0.000005). However, AMOVA based on predicted eye color grouping (brown, blue and undefined using a probability threshold of 0.7) resulted in an increased variance proportion of 48.7% (P<0.000005). Hence, although a considerable and significant proportion of genetic eye color variance is indeed explained by geography, about twice as much is explained when considering predicted eye color, as may be expected. Since the eye color prediction was solely based on the genetic variation (not using phenotype information), the AMOVA results highlight the presence of genetic homogeneity within each predicted eye color category.

To understand the geographic information content provided by the 6 eye color SNPs in more detail, we performed a non-metric multidimensional scaling (MDS) analysis of FST values estimated between pairs of all 51 HGDP-CEPH populations. As seen from the plot which was performed using k=2 dimensions (FIG. 6, S-Stress value 0.05998), all central, eastern and western European populations, which carry considerable amounts of predicted non-brown eyed individuals, cluster together and separately from all African, East Asian, Native American, Oceania populations as well as most of the Central South Asian and some Middle Eastern groups, i.e. all populations where brown was the only predicted eye color. The two southern European populations cluster together with the particular Middle Eastern and Pakistani groups who included low numbers of predicted non-brown eyed individuals; they all appeared somewhat between the non-southern Europeans on one side, and the remaining worldwide populations on the other side. Hence, on the population level, European geographic information can be inferred from the eye color SNPs used here (perhaps with the exception of southern Europe). However, on the individual level, which is of a greater concern in forensic applications, the situation appears different. No clear geographic clustering of individuals was evident in a MDS plot of identical-by-state distances obtained from the genotypes of the 6 eye color SNPs (data not shown). Noteworthy, European individuals are indeed differentiable from non-Europeans via hundreds of thousands of “random” SNPs [35, 36]. Also, European individuals, together with their neighbours from the Middle East and West Asia, can be differentiated from other worldwide individuals using small numbers of carefully ascertained ancestry-sensitive SNPs either obtained from regions outside pigmentation genes [37, 38], or applying a combination of markers from both pigmentation genes and other genomic regions [39, 40].

### Conclusions

Here we present a robust and sensitive DNA tool, termed IrisPlex™, for the accurate prediction of blue and brown eye color. The developed multiplex genotyping system based on the 6 currently most eye color informative SNPs i) allows prediction of blue and brown eye color with high levels of accuracy, ii) is extremely sensitive allowing successful analyses of picogram amounts of DNA, iii) is designed to cater for degraded DNA, and iv) is based on a genotyping technology that relies on equipment widely used by the forensic community. Hence, the IrisPlex™ system is highly suitable for application to forensic casework, including those with limited DNA quantity and quality. Our data from applying this system to eye color prediction on a worldwide scale provided supporting evidence that correct interpretation of blue and brown eye color prediction does not require additional ancestry information when this test and model are used. However, even considering this supporting evidence, it would still be of interest to perform a worldwide study on eye color prediction where phenotypic data is available. Also, future research into the genetic basis of non-blue and non-brown eye colors will need to show if such colors can be predicted with similarly high levels of accuracy as already possible for blue and brown eye color representing the two extremes of the continuous eye color distribution. As EVC prediction can create many new avenues of investigation combined with other means of intelligence, the IrisPlex™ eye color prediction system presented here is expected to become of great benefit to the forensic community in the coming years.

### REFERENCES

1. M. Kayser, P. M. Schneider, DNA-based prediction of human externally visible characteristics in forensics: Motivations, scientific challenges, and ethical considerations. Forensic Sci. Int. Genetics 3 (2009) 154-161.
2. P. Sulem, D. F. Gudbjartsson, S. N. Stacey, A. Helgason, T. Rafnar, K. P. Magnusson, A. Manolescu, A. Karason, A. Palsson, G. Thorleifsson, M. Jakobsdottir, S. Steinberg, S. Palsson, F. Jonasson, B. Sigurgeirsson, K. Thorisdottir, R. Ragnarsson, K. R. Benediktsdottir, K. K. Aben, L. A. Kiemeney, J. H. Olafsson, J. Gulcher, A. Kong, U. Thorsteinsdottir, K. Stefansson, Genetic determinants of hair, eye and skin pigmentation in Europeans. Nat. Genet. 39 (2007) 1443-1452.
3. H. Eiberg, J. Troelsen, M. Nielsen, A. Mikkelsen, J. Mengel-From, K. Kjaer, L. Hansen, Blue eye color in humans may be caused by a perfectly associated founder mutation in a regulatory element located within the HERC2 gene inhibiting OCA2 expression. Hum. Genet. 123 (2008) 177-187.
4. M. Kayser, F. Liu, A. C. J. W. Janssens, F. Rivadeneira, O. Lao, K. van Duijn, M. Vermeulen, P. Arp, M. M. Jhamai, W. F. J. van Ijcken, J. T. den Dunnen, S. Heath, D. Zelenika, D. D. G. Despriet, C. C. W. Klaver, J. R. Vingerling, P. T. V. M. de Jong, A. Hofman, Y. S. Aulchenko, A. G. Uitterlinden, B. A. Oostra, C. M. van Duijn, Three genome-wide association studies and a linkage analysis identify HERC2 as a human iris color gene. Am. J. Hum. Genet. 82 (2008) 411-423.
5. R. A. Sturm, D. L. Duffy, Z. Z. Zhao, F. P. N. Leite, M. S. Stark, N. K. Hayward, N. G. Martin, G. W. Montgomery, A single SNP in an evolutionary conserved region within intron 86 of the HERC2 gene determines human blue-brown eye color. Am. J. Hum. Genet. 82 (2008) 424-431.
6, J. Han, P. Kraft, H. Nan, Q. Guo, C. Chen, A. Qureshi, S. E. Hankinson, F. B. Hu, D. L. Duffy, Z. Z. Zhao, N. G. Martin, G. W. Montgomery, N. K. Hayward, G. Thomas, R. N. Hoover, S. Chanock, D. J. Hunter, A genome-wide association study identifies novel alleles associated with hair color and skin pigmentation. PLoS Genet. 4 (2008) e1000074.
7. P. Sulem, D. F. Gudbjartsson, S. N. Stacey, A. Helgason, T. Rafnar, M. Jakobsdottir, S. Steinberg, S. A. Gudjonsson, A. Palsson, G. Thorleifsson, S. Palsson, B. Sigurgeirsson, K. Thorisdottir, R. Ragnarsson, K. R. Benediktsdottir, K. K. Aben, S. H. Vermeulen, A. M. Goldstein, M. A. Tucker, L. A. Kiemeney, J. H. Olafsson, J. Gulcher, A. Kong, U. Thorsteinsdottir, K. Stefansson, Two newly identified genetic determinants of pigmentation in Europeans. Nat. Genet. 40 (2008) 835-837.
8. P. A. Kanetsky, J. Swoyer, S. Panossian, R. Holmes, D. Guerry, T. R. Rebbeck, A polymorphism in the Agouti signaling protein gene Is associated with human pigmentation. Am. J. Hum. Genet. 70 (2002) 770-775.
9. T. R. Rebbeck, P. A. Kanetsky, A. H. Walker, R. Holmes, A. C. Halpern, L. M. Schuchter, D. E. Elder, D. Guerry, P gene as an inherited biomarker of human eye color. Cancer Epidemiol. Biomarkers Prev. 11 (2002) 782-784.
10. F. Liu, K. van Duijn, J. R. Vingerling, A. Hofman, A. G. Uitterlinden, A. C. J. W. Janssens, M. Kayser, Eye color and the prediction of complex phenotypes from genotypes. Curr. Biol. 19 (2009) R192-R193.
11. R. L. Beals, H. Hoijer (1965) An introduction to anthropology. Macmillan, New York
12. R. A. Sturm, T. N. Frudakis, Eye color: portals into pigmentation genes and ancestry. Trends Genet. 20 (2004) 327-332.
13. P. Frost, European hair and eye color: A case of frequency-dependent sexual selection? Evol. Hum. Behav. 27 (2006) 85-103.
14. D. L. Duffy, G. W. Montgomery, W. Chen, Z. Z. Zhao, L. Le, M. R. James, N. K. Hayward, N. G. Martin, R. A. Sturm, A three-single-nucleotide polymorphism haplotype in intron 1 of OCA2 explains most human eye-color variation. Am. J. Hum. Genet. 80 (2007) 241-252.
15. G. Zhu, D. M. Evans, D. L. Duffy, G. W. Montgomery, S. E. Medland, N. A. Gillespie, K. R. Ewen, M. Jewell, Y. W. Liew, N. K. Hayward, R. A. Sturm, J. M. Trent, N. G. Martin, A genome scan for eye color in 502 twin families: most variation is due to a QTL on chromosome 15q. Twin Res. 7 (2004) 197-210.
16. D. Posthuma, P. M. Visscher, G. Willemsen, G. Zhu, N. G. Martin, P. E. Slagboom, E. J. de Geus, D. I. Boomsma, Replicated linkage for eye color on 15q using comparative ratings of sibling pairs. Behav. Genet. 36 (2006) 12-17.
17. T. N. Frudakis, M. Thomas, Z. Gaskin, K. Venkateswarlu, K. S. Chandra, S. Ginjupalli, S. Gunturi, S. Natrajan, V. K. Ponnuswamy, K. N. Ponnuswamy, Sequences associated with human iris pigmentation. Genetics 165 (2003) 2071-2083.
18. M. H. Brilliant, The mouse p (pink-eyed dilution) and human P genes, oculocutaneous albinism type 2 (OCA2), and melanosomal pH. Pigment Cell Res. 14 (2001) 86-93.
19. R. A. Sturm, Molecular genetics of human pigmentation diversity. Hum. Mol. Genet. 18 (2009) R9-17.
20. N. A. Rosenberg, Standardized subsets of the HGDP-CEPH Human Genome Diversity Cell Line Panel, accounting for atypical and duplicated samples and pairs of close relatives. Ann. Hum. Genet. 70 (2006) 841-847.
21. N. A. Rosenberg, J. K. Pritchard, J. L. Weber, H. M. Cann, K. K. Kidd, L. A. Zhivotovsky, M. W. Feldman, Genetic structure of human populations. Science 298 (2002) 2381-2385.
22. A. Untergasser, H. Nijveen, X. Rao, T. Bisseling, R. Geurts, J. A. M. Leunissen, Primer3Plus, an enhanced web interface to Primer3. Nucleic Acids Res. 35 (2007) W71-74.
23. S. F. Altschul, T. L. Madden, A. A. Schaffer, J. Zhang, Z. Zhang, W. Miller, D. J. Lipman, Gapped BLAST and PSI-BLAST: a new generation of protein database search programs. Nucleic Acids Res. 25 (1997) 3389-3402.
24. S. T. Sherry, M.-H. Ward, M. Kholodov, J. Baker, L. Phan, E. M. Smigielski, K. Sirotkin, dbSNP: the NCBI database of genetic variation. Nucleic Acids Res. 29 (2001) 308-311.
25. P. M. Vallone, J. M. Butler, AutoDimer: a screening tool for primer-dimer and hairpin structures. Biotechniques 37 (2004) 226-231.
26. B. S. Weir, C. Cockerham, Estimating F-Statistics for the Analysis of Population Structure. Evolution 38 (1984) 1358-1370.
27. L. Excoffier, P. E. Smouse, J. M. Quattro, Analysis of molecular variance inferred from metric distances among DNA haplotypes: application to human mitochondrial DNA restriction data. Genetics 131 (1992) 479-491.
28. L. Excoffier, G. Laval, S. Schneider, Arlequin (version 3.0): An integrated software package for population genetics data analysis. Evol. Bioinform. Online 1 (2005) 47-50.
29. J. J. Sanchez, C. Phillips, C. Børsting, K. Balogh, M. Bogus, M. Fondevila, C. D. Harrison, E. Musgrave-Brown, A. Salas, D. Syndercombe-Court, P. M. Schneider, A. Carracedo, N. Morling, A multiplex assay with 52 single nucleotide polymorphisms for human identification. Electrophoresis 27 (2006) 1713-1724.
30. L. A. Dixon, C. M. Murray, E. J. Archer, A. E. Dobbins, P. Koumi, P. Gill, Validation of a 21-locus autosomal SNP multiplex for forensic identification purposes. Forensic Sci. Int. 154 (2005) 62-77.
31. J. J. Mulero, C. W. Chang, R. E. Lagace, D. Y. Wang, J. L. Bas, T. P. McMahon, L. K. Hennessy, Development and validation of the AmpFISTR MiniFiler PCR Amplification Kit: A MiniSTR multiplex for the analysis of degraded and/or PCR inhibited DNA. J. Forensic Sci. 53 (2008) 838-852.
32. L. Z. Bito, A. Matheny, K. J. Cruickshanks, D. M. Nondahl, O. B. Carino, Eye color changes past early childhood: The Louisville Twin Study. Arch. Ophthalmol. 115 (1997) 659-663.
33. R. Iida, M. Ueki, H. Takeshita, J. Fujihara, T. Nakajima, Y. Kominato, M. Nagao, T. Yasuda, Genotyping of five single nucleotide polymorphisms in the OCA2 and HERC2 genes associated with blue-brown eye color in the Japanese population. Cell Biochem. Funct. 27 (2009) 323-327.
34. H. Pulker, M. V. Lareu, C. Phillips, A. Carracedo, Finding genes that underlie physical traits of forensic interest using genetic tools. Forensic Sci. Int. Genet. 1 (2007) 100-104.
35. J. Z. Li, D. M. Absher, H. Tang, A. M. Southwick, A. M. Casto, S. Ramachandran, H. M. Cann, G. S. Barsh, M. W. Feldman, L. L. Cavalli-Sforza, R. M. Myers, Worldwide human relationships inferred from genome-wide patterns of variation. Science 319 (2008) 1100-1104.
36. M. Jakobsson, S. W. Scholz, P. Scheet, J. R. Gibbs, J. M. VanLiere, H.-C. Fung, Z. A. Szpiech, J. H. Degnan, K. Wang, R. Guerreiro, J. M. Bras, J. C. Schymick, D. G. Hernandez, B. J. Traynor, J. Simon-Sanchez, M. Matarin, A. Britton, J. van de Leemput, I. Rafferty, M. Bucan, H. M. Cann, J. A. Hardy, N. A. Rosenberg, A. B. Singleton, Genotype, haplotype and copy-number variation in worldwide human populations. Nature 451 (2008) 998-1003.
37. O. Lao, K. van Duijn, P. Kersbergen, P. de Knijff, M. Kayser, Proportioning whole-genome single-nucleotide-polymorphism diversity for the identification of geographic population structure and genetic ancestry. Am. J. Hum. Genet. 78 (2006) 680-690.
38. P. Kersbergen, K. van Duijn, A. D. Kloosterman, J. T. den Dunnen, M. Kayser, P. de Knijff, Developing a set of ancestry-sensitive DNA markers reflecting continental origins of humans. BMC Genet. 10 (2009) 69.
39. C. Phillips, A. Salas, J. J. Sanchez, M. Fondevila, A. Gomez-Tato, J. Alvarez-Dios, M. Calaza, M. C. de Cal, D. Ballard, M. V. Lareu, A. Carracedo, Inferring ancestral origin using a single multiplex assay of ancestry-informative marker SNPs. Forensic Sci. Int. Genet. 1 (2007) 273-280.
40. D. Corach, O. Lao, C. Bobillo, K. van der Gaag, S. Zuniga, M. Vermeulen, K. van Duijn, M. Goedbloed, P. M. Vallone, W. Parson, P. de Knijff, M. Kayser, Inferring continental ancestry of Argentineans from autosomal, Y-chromosomal and mitochondrial DNA. Ann. Hum. Genet. 74 (2010) 65-76.

### Example 3

### Developmental Validation of the IrisPlex™ System

Developmental validation of the genotyping assay described in Example 2 has been conducted following the Scientific Working Group on DNA Analysis Methods (SWGDAM) guidelines for the application of DNA-based eye color prediction to forensic casework. This work is described in Walsh et al (2010) Forensic Sci Int: Genetics published online 12 Oct. 2010 (herein incorporated by reference in its entirety). The optimised assay conditions are described below.

### Multiplex Design & Protocol

The IrisPlex consists of 6 SNPs, rs12913832 (HERC2), rs1800407 (OCA2), rs12896399 (SLC24A4), rs16891982 (SLC45A2 (MATP)), rs1393350 (TYR) and rs12203592 (IRF4). PCR primers are as described in Example 2 and Table 9. SBE primer sequences and features, including slight alterations to the previously published sequences and features described in Example 2 are provided in Table 13. The protocol consists of a single multiplex two step PCR using 1 μl genomic DNA extract (varying concentrations) and primers in a 12 μl reaction which includes 1×PCR buffer, 2.7 mM MgCl₂, 200 μM of each dNTP and uses adjusted thermocycling conditions for increased specificity: (1) 95° C. for 10 min, (2) 33 cycles of 95° C. for 30 s and 61° C. for 30 s, (3) 5 min at 61° C. This is followed by product purification and a further multiplex single base extension (SBE) reaction using the ABI Prism® SNaPshot kit (Applied Biosystems) as described in Example 2. All cleaned products were analysed on the ABI 3130xl Genetic Analyser (Applied Biosystems) with POP-7 on a 36 cm capillary length array. Run parameters were optimised to increase sensitivity, with an injection voltage of 2.5 kV for 10 s, and run time of 500 s at 60° C.

### Multiplex Design & Protocol

The multiplex design of the IrisPlex assay was altered from the version in Example 2 in a bid to increase its sensitivity and specificity at the lower concentrations of DNA commonly found in casework samples. The annealing temperature of the multiplex PCR was increased slightly for improved specificity, and the SNP primer directions for rs1800407 and rs12203592 were altered in the subsequent SBE reaction to increase peak heights at lower template amounts. In the initial design, the reverse primer at rs12203592 caused a sporadic artefact that affected genotyping with low template levels. The use of the forward primer in the current protocol, giving a C/T genotype, avoids this problem. The change in primer direction of rs1800407 now produces increased peak heights with decreased primer input, which improves the call accuracy of this SNP at DNA amounts less than 250 pg. It is also easier to recognise heterozygote genotypes at this locus due to this increase in peak height. The primer concentration for SNP rs16891982 was also increased from 0.22 μM to 0.5 μM as the homozygote C/C allele was difficult to call with the previous protocol in low concentration DNA samples, due to its considerably lower peak height in comparison to the G/G homozygote allele. Notably, the increase in primer concentration creates a more balanced profile when the C/C allele is present. Finally, the ABI 3130xl Genetic Analysers' standard protocol was altered to increase detection sensitivity by increasing injection voltage and time, and to decrease overall processing time with a reduction to a 500 s run time. Overall, the slight changes made to the protocol described in Example 2 enhance the IrisPlex™ assay performance.

TABLE 8
PCR and extension primer sequences from Sequenom SNP genotyping

| | | 2nd- | | 1st- | |
|---|---|---|---|---|---|
| | | PCRP | | PCRP | |
| | | SEQ | | SEQ | |
| | | ID | | ID | |
| iPLEX | SNP | No. | Primer sequence | No. | |
| 1 | rs3794604 | | ACGTTGGATGATGCCCTCCTGGCTTTGTG | | ACGTTGGATGCACTTTTCTAGGGCTTTCAC |
| 1 | rs3935591 | | ACGTTGGATGACTGAGGTCCAGGTTCCTTG | | ACGTTGGATGTGGCTTTCGTGGAGGAACAG |
| 1 | rs4778232 | | ACGTTGGATGAACAGTTTCTTGCCCATGCC | | ACGTTGGATGAAGAACCAAGGGATCTAGGG |
| 1 | rs8041209 | | ACGTTGGATGAGAACTTGGTGGAGGATAGC | | ACGTTGGATGTCTTAGAGACAAAATTCCC |
| 1 | rs1667394 | | ACGTTGGATGCCATTAAGACGCAGCAATTC | | ACGTTGGATGGTCTTTTTCTCCTTTCAGTTC |
| 1 | rs16950987 | | ACGTTGGATGAATTACCCAGCATGCATGAC | | ACGTTGGATGCTTGTTACTTTATCTTCCTC |
| 1 | rs2346050 | | ACGTTGGATGGAGCCCAGCTGATTTTTCTC | | ACGTTGGATGGGAATTCTTCCACTTAATG |
| 1 | rs1800407 | | ACGTTGGATGACTCTGGCTTGTACTCTCTC | | ACGTTGGATGATGATGATCATGGCCCACAC |
| 1 | rs1129038 | | ACGTTGGATGCTTCTCATCAGACACACCAG | | ACGTTGGATGTCGTGAGATGAGAGCCTGAG |
| 1 | rs728405 | | ACGTTGGATGACCCCCATGGAAGAATGAGC | | ACGTTGGATGACATAGGATGCGTGAGTGTG |
| 1 | rs2240202 | | ACGTTGGATGTGGCCTCTTACAGGACTTAG | | ACGTTGGATGAGTCCTTTAAGCCCGGCTAC |
| 1 | rs12592730 | | ACGTTGGATGAGACAGAAAAGCTGCCAAG | | ACGTTGGATGATTCTGCTGTTATTGGCTGG |
| 1 | rs7179994 | | ACGTTGGATGGGCTCTAACCATAGCATCTC | | ACGTTGGATGCCAACAACCACACAGATGAG |
| 1 | rs7495174 | | ACGTTGGATGTAGGTCGGCTCCGTCG CAC | | ACGTTGGATGGGCTTAGGAAGCAAGGCAAG |
| 1 | rs1448485 | | ACGTTGGATGAGCTTCAGCAAGAGCCTAAC | | ACGTTGGATGCCCCACCATATTATTACCAG |
| 1 | rs7183877 | | ACGTTGGATGCTGTCTCATGGGTAGTAATC | | ACGTTGGATGACACTTGAAGCAGTATACA |
| 1 | rs683 | | ACGTTGGATGCCTTCTTTCTAATACAAGC | | ACGTTGGATGTTCTGAAAGGGTCTTCCCAG |
| 2 | rs8028689 | | ACGTTGGATGTTGTGCTGCTACTCATCTCC | | ACGTTGGATGAGTGCTAGCAATGCTAGGTC |
| 2 | rs12593929 | | ACGTTGGATGAGGACACCTGCCAGGACTAC | | ACGTTGGATGGAAGCACCTGAGAGTGTCTG |
| 2 | rs16891982 | | ACGTTGGATGTCTACGAAAGAGGAGTCGAG | | ACGTTGGATGAAAGTGAGGAAAACACGGAG |
| 2 | rs4778138 | | ACGTTGGATGCCTCCCATCACTGATTTAGC | | ACGTTGGATGGAAAGTCTCAAGGGAAATCAG |
| 2 | rs12896399 | | ACGTTGGATGGATGAGGAAGGTTAATCTGC | | ACGTTGGATGTCTGGCGATCCAATTCTTTG |
| 2 | rs4778241 | | ACGTTGGATGAGGAGTGCAATTGTTGGCTG | | ACGTTGGATGTGTACAGCCACTCTGGAAAG |
| 2 | rs916977 | | ACGTTGGATGTTCTGTTCTTCTTGACCCCG | | ACGTTGGATGGGTGTGGGATTTGTTTTGGC |
| 2 | 1512913832 | | ACGTTGGATGCGAGGCCAGTTTCATTTGAG | | ACGTTGGATGAAAACAAAGAGAAGCCTCGG |
| 2 | rs8024968 | | ACGTTGGATGCAGGGAGAGTACAGATTCAC | | ACGTTGGATGTTGGTGCCTTAGATGGACTG |
| 2 | rs16950979 | | ACGTTGGATGGCTCTGCTGCTCTTCTTCCA | | ACGTTGGATGAGGAAGCAGACGATAAGGAG |
| 2 | rs2240203 | | ACGTTGGATGTCTATATTAGCCTCATCAG | | ACGTTGGATGGAAGATCTTGCTTCCAAAGG |
| 2 | rs2594935 | | ACGTTGGATGGCCACACAACTTGGATCTTC | | ACGTTGGATGCCACAGGAAAACCTGCAATG |
| 2 | rs1597196 | | ACGTTGGATGAACTCTCCGTGCCTTCCTCC | | ACGTTGGATGGCATGAGTTCACGTGTATGA |
| 2 | rs1393350 | | ACGTTGGATGGGAAGGTGAATGATAACACG | | ACGTTGGATGTACTCTTCCTCAGTCCCTTC |
| 2 | rs26722 | | ACGTTGGATGGATGGAATGTACGAGTATGG | | ACGTTGGATGTTTTTGCTCCCTGCATTGCC |
| 2 | rs7170852 | | ACGTTGGATGATTTGTAGCAGCTGTGCGTC | | ACGTTGGATGACCAGGCCTTCTCTTTCATC |
| 2 | rs1635168 | | ACGTTGGATGAATCTCAGAGATCTTACCCG | | ACGTTGGATGACTTTGCCTGAGCACACAAG |
| | | SEQ | | | |
| | | ID | | | |
| iPLEX | SNP | No. | Extension primer sequence | | |
| 1 | rs3794604 | | GCTTTGTGGCCTCTCAC | | |
| 1 | rs3935591 | | TCCTTGCTGGCTGAGCTA | | |
| 1 | rs4778232 | | CTGCCCTCTTCTTCAACAG | | |
| 1 | rs8041209 | | TGGAGGATAGCCTACAGAT | | |
| 1 | rs1667394 | | AGCAATTCAAAACGTGCATA | | |
| 1 | rs16950987 | | tAGCATGCATGACTCATGAA | | |
| 1 | rs2346050 | | tTGATGACTTAGGGTTGGTG | | |
| 1 | rs1800407 | | cCCAGGCATACCGGCTCTCCC | | |
| 1 | rs1129038 | | CTACAGTCTACACAGCAGCGAG | | |
| 1 | rs728405 | | gGGAAGAATGAGCCAAAAAAAA | | |
| 1 | rs2240202 | | aCTCTTACAGGACTTAGTAACCGC | | |
| 1 | rs12592730 | | tACTGGATCCAATCAAAATTTACA | | |
| 1 | rs7179994 | | gaaggGTTCAGCTGGAGCAAGGTC | | |
| 1 | rs7495174 | | aTCCGTCGCACCCGTCTGTGCACACT | | |
| 1 | rs1448485 | | CCATGGTTGTTATTAATACTCATCAA | | |
| 1 | rs7183877 | | GGTAGTAATCAAAGAAACGACAAGTA | | |
| 1 | rs683 | | CTTCTTTCTAATACAAGCATATGTTAG | | |
| 2 | rs8028689 | | CTCAGTGTTCCACTTCC | | |
| 2 | rs12593929 | | GGGCCCACCTGCCACACG | | |
| 2 | rs16891982 | | GGTTGGATGTTGGGGCTT | | |
| 2 | rs4778138 | | CTGATTTAGCTGTGTTCTG | | |
| 2 | rs12896399 | | tgTCTGCTGTGACAAAGAGA | | |
| 2 | rs4778241 | | aggGGCTGGTAGTTGCAATT | | |
| 2 | rs916977 | | ttCAGCCTTGGCCAGCCTTCT | | |
| 2 | rs12913832 | | CCAGTTTCATTTGAGCATTAA | | |
| 2 | rs8024968 | | GAGAGTACAGATTCACAGACTT | | |
| 2 | rs16950979 | | gtttaCTCTTCTTCCAGCTCTTC | | |
| 2 | rs2240203 | | TGTCTTAATGTTTACATTCCTTA | | |
| 2 | rs2594935 | | TGGATCTTCTTGTAGCAAGTAAC | | |
| 2 | rs1597196 | | ccCAGGCTCTGGAACCTGCAATTT | | |
| 2 | rs1393350 | | ggtgGTAAAAGACCACACAGATTT | | |
| 2 | rs26722 | | gggagTGTACGAGTATGGTTCTATC | | |
| 2 | rs7170852 | | TTTGTAGCAGCTGTGCGTCTGTTTCC | | |
| 2 | rs1635168 | | cctccCAGAGATCTTACCCGTACCTGA | | |

TABLE 9
PCR primers included in the IrisPlex ™ system for eye color prediction

| | | | | | PCR |
|---|---|---|---|---|---|
| | Forward PCR primer (5'-3') | | Reverse PCR primer (5′-3′) | | Product |
| SNP-ID | SEQ ID No. | Primer sequence | SEQ ID No. | Primer sequence | (bp) |
| rs12913832 | | TGGCTCTCTGTGTCTGATCC | | GGCCCCTGATGATGATAGC | 87 |
| rs1800407 | | TGAAAGGCTGCCTCTGTTCT | | CGATGAGACAGAGCATGATGA | 127 |
| rs12896399 | | CTGGCGATCCAATTCTTTGT | | CTTAGCCCTGGGTCTTGATG | 104 |
| rs16891982 | | TCCAAGTTGTGCTAGACCAGA | | CGAAAGAGGAGTCGAGGTTG | 128 |
| rs1393350 | | TTCCTCAGTCCCTTCTCTGC | | GGGAAGGTGAATGATAACACG | 80 |
| rs12203592 | | ACAGGGCAGCTGATCTCTTC | | GCTAAACCTGGCACCAAAAG | 115 |

Primers are used at 0.416 μM.

TABLE 10
SBE primers included in the IrisPlex ™ system for eye color prediction

| | SEQ | | | | | |
|---|---|---|---|---|---|---|
| | ID | Extension Primer (5'-3') with | Primer | Conc. | Tm | Alleles |
| SNP-ID | No. | t-tail for length differentiation | Direction | (μM) | (° C.) | Detected |
| rs12913832 | | ttttttttttttttttttttttttGCGTGCAGAAC | Reverse | 0.2 | 55.0 | T/C |
| | | TTGACA | | | | |
| rs1800407 | | tttttttttCCCACACCCGTCCC | Reverse | 1.0 | 57.3 | C/T |
| rs12896399 | | tttttttttttttttttttttttttttttaTCTTT | Forward | 0.15 | 54.5 | G/T |
| | | AGGTCAGTATATTTTGGG | | | | |
| rs16891982 | | tttttttttttAAACACGGAGTTGATGCA | Forward | 0.22 | 55.9 | C/G |
| rs1393350 | | tttttttttttttttttttttttaTTTGTAAAAGA | Reverse | 0.1 | 55.6 | T/C |
| | | CCACACAGATTT | | | | |
| rs12203592 | | tttttttttttttttAAAGTACCACAGGGGAATTT | Reverse | 0.3 | 55.2 | G/A |

TABLE 11
α and β model parameters for 6 SNP eye color prediction

| | | | Blue | Intermediate | Eye color |
|---|---|---|---|---|---|
| | | | vs Brown | vs Brown | associated |
| | | | α1 | α2 | with |
| | Prediction | Minor | 3.94 | 0.65 | minor |
| SNP | Rank | Allele | β (π1) | β (π2) | allele |
| rs12913832 | 1 | A | −4.81 | −1.79 | Brown |
| rs1800407 | 2 | T | 1.40 | 0.87 | Blue |
| rs12896399 | 3 | G | −0.58 | −0.03 | Brown |
| rs16891982 | 4 | C | −1.30 | −0.50 | Brown |
| rs1393350 | 5 | A | 0.47 | 0.27 | Blue |
| rs12203592 | 6 | T | 0.70 | 0.73 | Blue |

TABLE 12
Actual eye color, sex, country of origin, and genotypes of the 6 SNPs included in the multiplex
tool together with derived eye color prediction probabilities of 40 individuals

| | |
|---|---|
| Actual | Inter- |

| | | | | | | | | | | | |
|---|---|---|---|---|---|---|---|---|---|---|---|
| eye | | Country of | | | | | | | Brown | mediate | Blue |
| color | Sex | Origin | rs12913832 | rs1800407 | rs12896399 | rs16891982 | rs1393350 | rs12203592 | (p) | (p) | (p) |

| | | | | | | | | | | | |
|---|---|---|---|---|---|---|---|---|---|---|---|
| blue | F | Netherlands | CC | CC | TT | GG | TT | GG | 0.01 | 0.02 | 0.97 |
| blue | F | New | CC | CC | TT | GG | CT | GG | 0.01 | 0.03 | 0.96 |
| | | Zealand | | | | | | | | | |
| blue | F | Netherlands | CC | CC | TT | GG | CT | GG | 0.01 | 0.03 | 0.96 |
| blue | M | Netherlands | CC | CC | TT | GG | CC | GA | 0.01 | 0.03 | 0.96 |
| blue | M | Netherlands | CC | CC | TT | GG | CC | GG | 0.02 | 0.03 | 0.95 |
| blue | F | Netherlands | CC | CC | TT | GG | CC | GG | 0.02 | 0.03 | 0.95 |
| blue | F | Netherlands | CC | CC | TT | GG | CC | GG | 0.02 | 0.03 | 0.95 |
| blue | F | Ireland | CC | CC | GT | GG | CT | AA | 0.01 | 0.05 | 0.94 |
| blue | M | Netherlands | CC | CC | GT | GG | CT | GG | 0.02 | 0.04 | 0.94 |
| blue | M | Netherlands | CC | CC | GT | GG | CT | GG | 0.02 | 0.04 | 0.94 |
| blue | F | Netherlands | CC | CC | GT | GG | CT | GG | 0.02 | 0.04 | 0.94 |
| blue | M | Estonia | CC | CC | GT | GG | CT | GG | 0.02 | 0.04 | 0.94 |
| blue | F | Netherlands | CC | CC | GG | GG | CT | GG | 0.03 | 0.07 | 0.9 |
| blue | F | Netherlands | CC | CC | GG | GG | CT | GG | 0.03 | 0.07 | 0.9 |
| blue | F | Netherlands | CC | CC | GG | GG | CT | GG | 0.03 | 0.07 | 0.9 |
| blue | M | Poland | CC | CC | GG | GG | CT | GG | 0.03 | 0.07 | 0.9 |
| blue | M | Netherlands | CC | CC | GG | GG | CT | GG | 0.03 | 0.07 | 0.9 |
| blue | M | Netherlands | CC | CC | GG | GG | CC | GG | 0.05 | 0.08 | 0.87 |
| blue | M | Netherlands | CC | CC | GG | GG | CC | GG | 0.05 | 0.08 | 0.87 |
| blue | M | Germany | CC | CC | GG | GG | CC | GG | 0.05 | 0.08 | 0.87 |
| blue | F | Germany | CC | CC | GG | GG | CC | GG | 0.05 | 0.08 | 0.87 |
| blue | M | Russia | CC | CC | GG | GG | CC | GG | 0.05 | 0.08 | 0.87 |
| blue | M | Ireland | CT | CT | GG | GG | CT | AA | 0.17 | 0.49 | 0.34 |
| blue | M | Netherlands | CT | CC | GG | GG | CT | GG | 0.69 | 0.18 | 0.13 |
| brown | M | Spain | CT | CT | TT | GG | CC | GG | 0.36 | 0.22 | 0.42 |
| brown | F | Netherlands | CT | CT | GT | GG | CC | GG | 0.45 | 0.25 | 0.3 |
| brown | M | Spain | CT | CC | TT | GG | CT | GG | 0.55 | 0.14 | 0.31 |
| brown | M | Netherlands | CT | CC | TT | GG | CT | GG | 0.55 | 0.14 | 0.31 |
| brown | M | Netherlands | CT | CC | TT | GG | CC | GG | 0.64 | 0.13 | 0.23 |
| brown | M | Netherlands | CT | CC | TT | GG | CC | GG | 0.64 | 0.13 | 0.23 |
| brown | M | Netherlands | CT | CC | GG | GG | CT | GG | 0.69 | 0.18 | 0.13 |
| brown | F | Portugal | CT | CC | GG | GG | CC | GG | 0.76 | 0.15 | 0.09 |
| brown | F | Netherlands | CT | CC | GG | GG | CC | GG | 0.76 | 0.15 | 0.09 |
| brown | F | Serbia | TT | CC | GG | GG | CT | GA | 0.93 | 0.06 | 0.01 |
| brown | M | Iran | TT | CC | GG | GG | TT | GG | 0.96 | 0.04 | 0 |
| brown | M | Turkey | TT | CC | GG | GG | CT | GG | 0.97 | 0.03 | 0 |
| brown | F | Suriname | TT | CC | GG | CC | CC | GG | 0.97 | 0.03 | 0 |
| brown | F | Suriname | TT | CC | GG | CC | CC | GG | 0.99 | 0.01 | 0 |
| brown | F | Suriname | TT | CC | GT | CC | CC | GG | 0.99 | 0.01 | 0 |
| brown | F | China | TT | CC | GG | CC | CC | GG | 0.99 | 0.01 | 0 |

TABLE 13
SBE primers included in the developmentally
validated IrisPlex ™ system for eye color prediction

| | SEQ ID | Extension Primer (5′-3′) with t-tail | Primer | Conc. | Tm | Alleles |
|---|---|---|---|---|---|---|
| SNP-ID | No. | for length differentiation | Direction | (μM) | (° C.) | Detected |
| rs12913832 | | tttttttttttttttttttttttGCGTGCAGAACTTGACA | Reverse | 0.2 | 55.0 | T/C |
| rs1800407 | | tttttttGCATACCGGCTCTCCC | Forward | 0.1 | 57.3 | G/A |
| rs12896399 | | tttttttttttttttttttttttttttttaTCTTTAGGTCAGTATATTTTGGG | Forward | 0.15 | 54.5 | G/T |
| rs16891982 | | tttttttttttAAACACGGAGTTGATGCA | Forward | 0.5 | 55.9 | C/G |
| rs1393350 | | tttttttttttttttttttttttaTTTGTAAAAGACCACACAGATTT | Reverse | 0.1 | 55.6 | T/C |
| rs12203592 | | tttttttttttttttaTTTGGTGGGTAAAAGAAGG | Forward | 0.3 | 55.2 | C/T |

Changes compared to the corresponding information in Table 9 are shown in bold.

1. A method for predicting the iris color of a human, the method comprising:
(a) obtaining a sample of the nucleic acid of the human;
(b) genotyping the nucleic acid for at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5; and
(c) predicting the iris color based on the results of step (b).

2. The method of claim 1 wherein:
the polymorphic site which is in linkage disequilibrium with rs12913832 at an r² value of at least 0.9 is rs1129038;
the polymorphic site which is in linkage disequilibrium with rs1800407 at an r² value of at least 0.5 is selected from the group consisting of rs9920172, rs11638265, rs1800411, rs1448488, rs11636005, rs11634923, rs7182323, rs11631735, rs12914687, rs12903382, rs12910433, rs1900758, rs11630828, rs7178315, rs735067, rs2015343, rs8029026, rs2077596, rs8024822 and rs11636259; and
the polymorphic site which is in linkage disequilibrium with rs12896399 at an r² value of at least 0.5 is selected from the group consisting of rs8017054, rs4900109, rs4904866, rs746586, rs1075830, rs941799, rs1885194, rs17184180, rs4904868, rs4904870 and rs4900114.

3. The method of claim 1 wherein step (b) further comprises genotyping the nucleic acid for at least one polymorphism selected from the group consisting of:
(i) the SNP rs16891982 or a polymorphic site which is in linkage disequilibrium with rs16891982 at an r² value of at least 0.5;
(ii) the SNP rs1393350 or a polymorphic site which is in linkage disequilibrium with rs1393350 at an r² value of at least 0.5;
(iii) the SNP rs12203592 or a polymorphic site which is in linkage disequilibrium with rs12203592 at an r² value of at least 0.5;

4. The method of claim 3 wherein:
the polymorphic site which is in linkage disequilibrium with rs16891982 at an r² value of at least 0.5 is selected from the group consisting of rs35407, rs35395, rs35397, rs2278007, rs35389, rs28777, rs183671 and rs3797201; and
the polymorphic site which is in linkage disequilibrium with rs1393350 at an r² value of at least 0.5 is selected from the group consisting of rs10765198, rs7358418, rs10765200, rs10765201, rs4396293, rs2186640, rs10501698, rs10830250, rs7924589, rs4121401, rs1847134, rs1827430, rs3900053, rs1847142, rs4121403, rs10830253, rs7951935, rs1847140, rs1806319, rs4106039, rs4106040, rs11018463, rs11018464, rs12363323, rs1942486, rs17792911, rs10830219, rs10830236, rs12270717, rs7129973, rs11018525, rs17793678, rs10765196, rs10765197, rs7123654, rs11018528, rs12791412, rs12789914, rs7107143, rs4512823, rs4512825, rs7101897 and rs1126809.

5. The method of claim 3 wherein step (b) further comprises genotyping the nucleic acid for each polymorphism.

6. The method of claim 5 wherein step (b) further comprises genotyping the nucleic acid for at least one polymorphism selected from the group consisting of:
(i) the SNP rs12592730 or a polymorphic site which is in linkage disequilibrium with rs12592730 at an r² value of at least 0.5;
(ii) the SNP rs7495174 or a polymorphic site which is in linkage disequilibrium with rs7495174 at an r² value of at least 0.5;
(iv) the SNP rs1667394 or a polymorphic site which is in linkage disequilibrium with rs1667394 at an r² value of at least 0.5;
(iv) the SNP rs7183877 or a polymorphic site which is in linkage disequilibrium with rs7183877 at an r² value of at least 0.5;
(v) the SNP rs4778232 or a polymorphic site which is in linkage disequilibrium with rs4778232 at an r² value of at least 0.5;
(vi) the SNP rs1408799 or a polymorphic site which is in linkage disequilibrium with rs1408799 at an r² value of at least 0.5;
(vii) the SNP rs8024968 or a polymorphic site which is in linkage disequilibrium with rs8024968 at an r² value of at least 0.5;
(viii) the SNP rs683 or a polymorphic site which is in linkage disequilibrium with rs683 at an r² value of at least 0.5.

7. The method of claim 1 wherein step (c) comprises a categorical prediction of the iris color.

8. The method of claim 7 wherein the categorical prediction is of brown, blue or intermediate.

9. The method of claim 1 wherein for each polymorphism to be genotyped in step (b), the method comprises contacting the sample of the nucleic acid of the human with a nucleic acid molecule that hybridizes selectively to a genomic region encompassing the polymorphism.

10. The method of claim 9 wherein the sample of the nucleic acid of the human is subjected to a nucleic acid amplification before being contacted with the nucleic acid molecule.

11. The method of claim 9 or 10 wherein the nucleic acid molecule is a primer and the method comprises performing a primer extension reaction and detecting the primer extension reaction product.

12. The method of claim 11 wherein the primer extension reaction is a multiplex primer extension reaction.

13. A method of preparing a data carrier containing data on the predicted iris color of a human, the method comprising carrying out the method of claim 1 and recording the results on a data carrier.

14. A method of preparing a data carrier containing data on the predicted iris color of a human, the method comprising recording the results of a method carried out according to claim 1 on a data carrier.

15. The method of claim 13 or 14 wherein the data is recorded in electronic form.

16. A method for predicting the iris color of a human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

17. A method for creating a description of a human based on forensic testing, wherein the description includes a prediction of the iris color of the human based on the allele occurrences in a sample of their DNA of at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

18. A method for genotyping polymorphisms indicative of human iris color comprising:
(a) obtaining a sample of the nucleic acid of a human; and
(b) genotyping the nucleic acid for at least the following polymorphisms:
(i) the single nucleotide polymorphism (SNP) rs12913832 or a polymorphic site which is in linkage disequilibrium with rs12913832 at an r² value of at least 0.9;
(ii) the SNP rs1800407 or a polymorphic site which is in linkage disequilibrium with rs1800407 at an r² value of at least 0.5; and,
(iii) the SNP rs12896399 or a polymorphic site which is in linkage disequilibrium with rs12896399 at an r² value of at least 0.5.

19. A kit of parts for use in predicting the iris color of a human comprising:
(i) a primer pair suitable for amplifying the genomic region encompassing the single nucleotide polymorphism (SNP) rs12913832 or a polymorphic site which is in linkage disequilibrium with rs12913832 at an r² value of at least 0.9;
(ii) a primer pair suitable for amplifying the genomic region encompassing the SNP rs1800407 or a polymorphic site which is in linkage disequilibrium with rs1800407 at an r² value of at least 0.5; and,
(iii) a primer pair suitable for amplifying the genomic region encompassing the SNP rs12896399 or a polymorphic site which is in linkage disequilibrium with rs12896399 at an r² value of at least 0.5.

20. The kit of claim 19 wherein each of the primer pairs are suitable for use together in a multiplex polymerase chain reaction.

21. A kit of parts for use in predicting the iris color of a human comprising:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

22. The kit of claim 21 wherein each of the nucleic acid molecules is a primer suitable for performing a primer extension reaction.

23. A solid substrate for use in predicting the iris color of a human, the solid substrate having attached thereto:
(i) a nucleic acid molecule that hybridizes selectively to a genomic region encompassing the single nucleotide polymorphism (SNP) rs12913832 or a polymorphic site which is in linkage disequilibrium with rs12913832 at an r² value of at least 0.9;
(ii) a nucleic acid molecule that hybridizes selectively to a genomic region encompassing the SNP rs1800407 or a polymorphic site which is in linkage disequilibrium with rs1800407 at an r² value of at least 0.5; and,
(iii) a nucleic acid molecule that hybridizes selectively to a genomic region encompassing the SNP rs12896399 or a polymorphic site which is in linkage disequilibrium with rs12896399 at an r² value of at least 0.5.

24. The solid substrate of claim 23 wherein each of the nucleic acid molecules is a primer suitable for performing a primer extension reaction.
